(19) United States
(12) Patent Application Publication
(10) Pub. No.: US 2014/0343865 A1
(43) Pub. Date: Nov. 20, 2014

(54) Biomarkers for Kidney Cancer and Methods Using the Same

(71) Applicants: Meredith V. Brown, Durham, NC (US); Kay A. Lawton, Raleigh, NC (US); Bruce Neri, Cary, NC (US); Yang Chen, Salt Lake City, UT (US)

(72) Inventors: Meredith V. Brown, Durham, NC (US); Kay A. Lawton, Raleigh, NC (US); Bruce Neri, Cary, NC (US); Yang Chen, Salt Lake City, UT (US)

(21) Appl. No.: 14/362,943

(22) PCT Filed: Dec. 7, 2012
(86) PCT No.: PCT/US12/68506

§ 371 Date: Jun. 5, 2014

Related U.S. Application Data
(60) Provisional application No. 61/568,690, filed on Dec. 9, 2011.
(60) Provisional application No. 61/677,771, filed on Jul. 31, 2012.

(51) Int. Cl. G01N 33/574; G06F 19/12

(52) U.S. Cl. CPC G01N 33/57438; G06F 19/12
USPC 702/19; 435/7.92; 436/501

(57) ABSTRACT

Methods for identifying and evaluating biochemical entities useful as biomarkers for kidney cancer, target identification/validation, and monitoring of drug efficacy are provided. Also provided are suites of small molecule entities as biomarkers for kidney cancer.

This application claims the benefit of U.S. Provisional Patent Application No. 61/568,690, filed Dec. 9, 2011, and U.S. Provisional Patent Application No. 61/677,771, filed Jul. 31, 2012, the entire contents of which are hereby incorporated herein by reference.

### FIELD

The invention generally relates to biomarkers for kidney cancer and methods based on the same biomarkers.

### BACKGROUND

In the US, 275,000 patients each year are screened for kidney cancer, and 55,000 are diagnosed with renal cell carcinoma (RCC) (American Cancer Society Facts and Figures 2010). RCC is the most common form of kidney cancer, accounting for approximately 80% of the total. The incidence of RCC is steadily increasing, and in the US increased by approximately 2% per year in the past two decades (Ries L A G, et al., eds. SEER Cancer Statistics Review, 1975-2003. Bethesda, Md.: National Cancer Institute; 2006). Because RCC is one of the deadliest cancers and does not respond to traditional chemotherapy drugs, many new targeted agents are being developed specifically to treat RCC.

70% of newly diagnosed patients are diagnosed in the early stages (T1 and T2). Early stage RCC is treated by partial or total nephrectomy; this is surgery with curative intent. When RCC tumors are surgically removed at an early stage, the 5 year survival rate is 90% for stage 1 and 51% for stage 2, yet 70% of RCC patients develop metastasis during the course of their disease.

Often, kidney lesions or small renal masses (SRM) are discovered incidentally during examinations unrelated to suspected malignancy. While approximately 20% of SRM are benign, the remainder are cancerous. The traditional treatment for small renal masses is radical nephrectomy. Typically cancer-positive SRMs are relatively small and have a relatively slow growth rate. As such, cancer-positive SRMs are generally considered to have less aggressive potential, and thus a watchful waiting approach may be more appropriate than surgery (Bosniak M A, et al. J. Small renal parenchymal neoplasms: further observations on growth. Radiology 1995; 197: 589-597.). However, there are also incidentally detected small renal masses that can grow rapidly and have aggressive potential (Remzi M, et al. “Are small renal tumors harmless? Analysis of histopathological features according to tumors 4 cm or less in diameter”. J. Urol. 2006; 176 (3): 896-9.). Biomarkers for distinguishing which cancer-positive SRMs will be more aggressive, requiring surgery, and which will be slower growing and warrant a watchful waiting approach would be valuable.

Pharmaceutical companies have been developing targeted therapies for RCC, such as Sutent (sunitinib), Nexavar (sorafenib), Avastin (bevacizumab) and Torisel (temsirolimus). As of March 2011, there were 6 targeted agents in Phase I, 13 in Phase 2, 5 in Phase 3, and 8 with FDA approval for treatment of RCC. Currently, approximately 18% of the RCC patient population receives drug therapy. In the future, more patients are expected to receive treatment, driven by an increase in the number of treatment options, improvements in drug efficacy and the trend to use drug therapy earlier in the course of the disease (adjuvant or neo-adjuvant setting) (Espicom Business Intelligence, Market Report: Renal Cell Carcinoma Drug Futures, ISBN: 978-1-85822-396-4, March 2011).

### SUMMARY

In one aspect, the present invention provides a method of diagnosing whether a subject has kidney cancer, including subjects having an SRM, comprising analyzing a biological sample from a subject to determine the level(s) of one or more biomarkers for kidney cancer in the sample, where the one or more biomarkers are selected from Tables 1, 2, 4 and/or 11 and comparing the level(s) of the one or more biomarkers in the sample to kidney cancer-positive and/or kidney cancer-negative reference levels of the one or more biomarkers in order to diagnose whether the subject has kidney cancer.

In a further aspect, the invention provides a method of distinguishing kidney cancer from other urological cancers (e.g., bladder cancer, prostate cancer), comprising analyzing a biological sample from a subject to determine the level(s) of one or more biomarkers for kidney cancer in the sample where the one or more biomarkers are selected from Table 11 and comparing the level(s) of the one or more biomarkers in the sample to kidney cancer-positive and/or kidney cancer-negative reference levels of the one or more biomarkers in order to distinguish kidney cancer from other urological cancers.

In another aspect, the invention provides a method of monitoring progression/regression of kidney cancer in a subject comprising analyzing a first biological sample from a subject to determine the level(s) of one or more biomarkers for kidney cancer in the sample, where the one or more biomarkers are selected from Tables 1, 2, 4, 8, 10 and/or 11 and the first sample is obtained from the subject at a first time point; analyzing a second biological sample from a subject to determine the level(s) of the one or more biomarkers, where the second sample is obtained from the subject at a second time point; and comparing the level(s) of one or more biomarkers in the second sample to the level(s) of the one or more biomarkers in (a) the first sample (b) kidney cancer-positive reference levels of the one or more biomarkers, and/or (c) kidney cancer-negative reference levels of the one or more biomarkers in order to monitor the progression/regression of kidney cancer in the subject.

In another aspect, the present invention provides a method of determining the stage of kidney cancer, comprising analyzing a biological sample from a subject to determine the level(s) of one or more biomarkers for kidney cancer stage in the sample, where the one or more biomarkers are selected from Table 8; and comparing the level(s) of the one or more biomarkers in the sample to high stage kidney cancer and/or low stage kidney cancer reference levels of the one or more biomarkers in order to determine the stage of the subject's kidney cancer.

In a further aspect, the present invention provides a method of determining the aggressiveness of kidney cancer, comprising analyzing a biological sample from a subject to determine the level(s) of one or more biomarkers for kidney cancer aggressiveness in the sample, where the one or more biomarkers are selected from Table 10; and comparing the level(s) of the one or more biomarkers in the sample to more aggressive kidney cancer and/or less aggressive kidney cancer reference levels of the one or more biomarkers in order to determine the aggressiveness of the subject's kidney cancer.

In another aspect, the present invention provides a method of assessing the efficacy of a composition for treating kidney cancer comprising analyzing a biological sample from a subject having kidney cancer and currently or previously being treated with the composition, to determine the level(s) of one or more biomarkers for kidney cancer selected from Tables 1, 2, 4, 8, 10 and/or 11; and comparing the level(s) of the one or more biomarkers in the sample to (a) levels of the one or more biomarkers in a previously-taken biological sample from the subject, where the previously-taken biological sample was obtained from the subject before being treated with the composition, (b) kidney cancer-positive reference levels of the one or more biomarkers, and/or (c) kidney cancer-negative reference levels of the one or more biomarkers.

In another aspect, the present invention provides a method for assessing the efficacy of a composition in treating kidney cancer, comprising analyzing a first biological sample from a subject to determine the level(s) of one or more biomarkers for kidney cancer selected from Tables 1, 2, 4, 8, 10 and/or 11, the first sample obtained from the subject at a first time point; administering the composition to the subject; analyzing a second biological sample from the subject to determine the level(s) of the one or more biomarkers, the second sample obtained from the subject at a second time point after administration of the composition; comparing the level(s) of one or more biomarkers in the first sample to the level(s) of the one or more biomarkers in the second sample in order to assess the efficacy of the composition for treating kidney cancer.

In yet another aspect, the invention provides a method of assessing the relative efficacy of two or more compositions for treating kidney cancer comprising analyzing, from a first subject having kidney cancer and currently or previously being treated with a first composition, a first biological sample to determine the level(s) of one or more biomarkers selected from Tables 1, 2, 4, 8, 10 and/or 11; analyzing, from a second subject having kidney cancer and currently or previously being treated with a second composition, a second biological sample to determine the level(s) of the one or more biomarkers; and comparing the level(s) of one or more biomarkers in the first sample to the level(s) of the one or more biomarkers in the second sample in order to assess the relative efficacy of the first and second compositions for treating kidney cancer.

In another aspect, the present invention provides a method for screening a composition for activity in modulating one or more biomarkers of kidney cancer, comprising contacting one or more cells with a composition; analyzing at least a portion of the one or more cells or a biological sample associated with the cells to determine the level(s) of one or more biomarkers of kidney cancer selected from Tables 1, 2, 4, 8, 10 and/or 11; and comparing the level(s) of the one or more biomarkers with predetermined standard levels for the biomarkers to determine whether the composition modulated the level(s) of the one or more biomarkers.

In yet another aspect, the invention provides a method for treating a subject having kidney cancer comprising administering to the subject an effective amount of one or more biomarkers selected from Tables 1, 2, 4, 8, 10 and/or 11 that are decreased in kidney cancer.

### BRIEF DESCRIPTION OF THE DRAWINGS

FIG. 1. Graphical illustration of feature-selected principal components analysis (PCA) using biopsy tissue from kidney cancer and benign samples. An arbitrary cutoff line is drawn to illustrate that these metabolic abundance profiles can separate samples into groups with both high Negative Predictive Value (NPV) (PC1<0) and high Positive Predictive Value (PPV) (PC1>0).

FIG. 2. Graphical illustration of feature-selected hierarchical clustering (Euclidean distance) using biopsy tissue from kidney cancer and benign samples. Two distinct metabolic classes were identified, one containing 80% kidney cancer samples and one containing 71% benign samples.

### DETAILED DESCRIPTION

The present invention relates to biomarkers of kidney cancer, methods for diagnosis or aiding in diagnosis of kidney cancer, methods of determining or aiding in determining the cancer status of a small renal mass (SRM) kidney cancer, methods of staging kidney cancer, methods of determining kidney cancer aggressiveness, methods of monitoring progression/regression of kidney cancer, methods of assessing efficacy of compositions for treating kidney cancer, methods of screening compositions for activity in modulating biomarkers of kidney cancer, methods of treating kidney cancer, as well as other methods based on biomarkers of kidney cancer. Prior to describing this invention in further detail, however, the following terms will first be defined.

### DEFINITIONS

“Biomarker” means a compound, preferably a metabolite, that is differentially present (i.e., increased or decreased) in a biological sample from a subject or a group of subjects having a first phenotype (e.g., having a disease) as compared to a biological sample from a subject or group of subjects having a second phenotype (e.g., not having the disease). A biomarker may be differentially present at any level, but is generally present at a level that is increased by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, by at least 100%, by at least 110%, by at least 120%, by at least 130%, by at least 140%, by at least 150%, or more; or is generally present at a level that is decreased by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, or by 100% (i.e., absent). A biomarker is preferably differentially present at a level that is statistically significant (i.e., a p-value less than 0.05 and/or a q-value of less than 0.10 as determined using either Welch's T-test or Wilcoxon's rank-sum Test).

The “level” of one or more biomarkers means the absolute or relative amount or concentration of the biomarker in the sample.

“Sample” or “biological sample” means biological material isolated from a subject. The biological sample may contain any biological material suitable for detecting the desired biomarkers, and may comprise cellular and/or non-cellular material from the subject. The sample can be isolated from any suitable biological tissue or fluid such as, for example, kidney tissue, blood, blood plasma, urine, or cerebral spinal fluid (CSF).

“Subject” means any animal, but is preferably a mammal, such as, for example, a human, monkey, mouse, rabbit or rat.

A “reference level” of a biomarker means a level of the biomarker that is indicative of a particular disease state, phenotype, or lack thereof, as well as combinations of disease states, phenotypes, or lack thereof. A “positive” reference level of a biomarker means a level that is indicative of a particular disease state or phenotype. A “negative” reference level of a biomarker means a level that is indicative of a lack of a particular disease state or phenotype. For example, a “kidney cancer-positive reference level” of a biomarker means a level of a biomarker that is indicative of a positive diagnosis of kidney cancer in a subject, and a “kidney cancer-negative reference level” of a biomarker means a level of a biomarker that is indicative of a negative diagnosis of kidney cancer in a subject. A “reference level” of a biomarker may be an absolute or relative amount or concentration of the biomarker, a presence or absence of the biomarker, a range of amount or concentration of the biomarker, a minimum and/or maximum amount or concentration of the biomarker, a mean amount or concentration of the biomarker, and/or a median amount or concentration of the biomarker; and, in addition, “reference levels” of combinations of biomarkers may also be ratios of absolute or relative amounts or concentrations of two or more biomarkers with respect to each other. Appropriate positive and negative reference levels of biomarkers for a particular disease state, phenotype, or lack thereof may be determined by measuring levels of desired biomarkers in one or more appropriate subjects, and such reference levels may be tailored to specific populations of subjects (e.g., a reference level may be age-matched so that comparisons may be made between biomarker levels in samples from subjects of a certain age and reference levels for a particular disease state, phenotype, or lack thereof in a certain age group). Such reference levels may also be tailored to specific techniques that are used to measure levels of biomarkers in biological samples (e.g., LC-MS, GC-MS, etc.), where the levels of biomarkers may differ based on the specific technique that is used.

“Non-biomarker compound” means a compound that is not differentially present in a biological sample from a subject or a group of subjects having a first phenotype (e.g., having a first disease) as compared to a biological sample from a subject or group of subjects having a second phenotype (e.g., not having the first disease). Such non-biomarker compounds may, however, be biomarkers in a biological sample from a subject or a group of subjects having a third phenotype (e.g., having a second disease) as compared to the first phenotype (e.g., having the first disease) or the second phenotype (e.g., not having the first disease).

“Metabolite”, or “small molecule”, means organic and inorganic molecules which are present in a cell. The term does not include large macromolecules, such as large proteins (e.g., proteins with molecular weights over 2,000, 3,000, 4,000, 5,000, 6,000, 7,000, 8,000, 9,000, or 10,000), large nucleic acids (e.g., nucleic acids with molecular weights of over 2,000, 3,000, 4,000, 5,000, 6,000, 7,000, 8,000, 9,000, or 10,000), or large polysaccharides (e.g., polysaccharides with a molecular weights of over 2,000, 3,000, 4,000, 5,000, 6,000, 7,000, 8,000, 9,000, or 10,000). The small molecules of the cell are generally found free in solution in the cytoplasm or in other organelles, such as the mitochondria, where they form a pool of intermediates which can be metabolized further or used to generate large molecules, called macromolecules. The term “small molecules” includes signaling molecules and intermediates in the chemical reactions that transform energy derived from food into usable forms. Examples of small molecules include sugars, fatty acids, amino acids, nucleotides, intermediates formed during cellular processes, and other small molecules found within the cell.

“Metabolic profile”, or “small molecule profile”, means a complete or partial inventory of small molecules within a targeted cell, tissue, organ, organism, or fraction thereof (e.g., cellular compartment). The inventory may include the quantity and/or type of small molecules present. The “small molecule profile” may be determined using a single technique or multiple different techniques.

“Metabolome” means all of the small molecules present in a given organism.

“Kidney cancer” refers to a disease in which cancer develops in the kidney.

“Urological Cancer” refers to a disease in which cancer develops in the bladder, kidney and/or prostate.

“Staging” of kidney cancer refers to an indication of the severity of kidney cancer including tumor size and whether and/or how far the kidney tumor has spread. The tumor stage is a criteria used to select treatment options and to estimate a patient's prognosis. Kidney tumor stages range from T1 (tumor 7 cm or less in size and limited to kidney, least advanced) to T4 (tumor invades beyond Gerota's fascia, most advanced). “Low stage” or “lower stage” kidney cancer refers to kidney cancer tumors, including malignant tumors with a lower potential for recurrence, progression, invasion and/or metastasis (less advanced). Kidney tumors of stage T1 or T2 are considered “low stage”. “High stage” or “higher stage” kidney cancer refers to a kidney cancer tumor in a subject that is more likely to recur and/or progress and/or invade beyond the kidney, including malignant tumors with higher potential for metastasis (more advanced). Kidney tumors of stage T3 or T4 are considered “high stage”.

“Grade” of kidney cancer refers to the appearance and/or structure of kidney cancer cellular nuclei. “Low grade” kidney cancer refers to a cancer with cellular nuclear characteristics more closely resembling normal cellular nuclei. “High grade” kidney cancer refers to a cancer with cellular nuclear characteristics less closely resembling normal cellular nuclei.

“Aggressiveness” of kidney cancer or a cancer-positive small renal mass refers to a combination of the stage, grade, and metastatic potential of a kidney tumor. “More aggressive” kidney cancer refers to tumors of higher stage, grade, and/or metastatic potential. Cancer tumors that are not confined to the kidney are considered to be more aggressive kidney cancer. “Less aggressive” kidney cancer refers to tumors of lower stage, grade, and/or metastatic potential. Cancer tumors that are confined to the kidney are considered to be less aggressive kidney cancer.

“Small renal mass (SRM)” refers to a kidney lesion that may be detected incidentally during an examination but is usually not yet associated with symptoms of kidney cancer. The SRM may be benign (cancer-negative) or may be a cancer tumor (cancer-positive). A cancer-positive SRM may be an indolent tumor (low stage/less aggressive) or may be a high stage, aggressive tumor.

“RCC Score” is a measure or indicator of kidney cancer severity, which is based on the kidney cancer biomarkers and algorithms described herein. An RCC Score will enable a physician to place a patient on a spectrum of kidney cancer severity from normal (i.e., no kidney cancer) to high (e.g., high stage or more aggressive kidney cancer). One of ordinary skill in the art will understand that the RCC Score can have multiple uses in the diagnosis and treatment of kidney cancer. For example, an RCC Score may also be used to distinguish less aggressive kidney cancer from more aggressive kidney cancer, to distinguish low grade kidney cancer from high grade kidney cancer, and to monitor the progression and/or regression of kidney cancer.

### I. BIOMARKERS

The kidney cancer biomarkers described herein were discovered using metabolomic profiling techniques. Such metabolomic profiling techniques are described in more detail in the Examples set forth below as well as in U.S. Pat. Nos. 7,005,255, 7,329,489; 7,550,258; 7,550,260; 7,553,616; 7,635,556; 7,682,783; 7,682,784; 7,910,301; 6,947,453; 7,433,787; 7,561,975; 7,884,318, the entire contents of which are hereby incorporated herein by reference.

Generally, metabolic profiles were determined for biological samples from human subjects that were positive for kidney cancer (RCC) or samples from human subjects that were cancer negative (non-cancer). The metabolic profile for biological samples positive for kidney cancer was compared to the metabolic profile for biological samples negative for kidney cancer. Those small molecules differentially present, including those small molecules differentially present at a level that is statistically significant, in the metabolic profile of samples positive for kidney cancer as compared to another group (e.g., non-cancer samples) were identified as biomarkers to distinguish those groups.

The biomarkers are discussed in more detail herein. The biomarkers that were discovered correspond with biomarkers for distinguishing samples positive for kidney cancer (RCC) vs. cancer-negative samples (see Tables 1, 2, 4 and/or 11).

Metabolic profiles were also determined for biological samples from human subjects diagnosed with high stage kidney cancer or human subjects diagnosed with low stage kidney cancer. The metabolic profile for biological samples from a subject having high stage kidney cancer was compared to the metabolic profile for biological samples from subjects with low stage kidney cancer. Those small molecules differentially present, including those small molecules differentially present at a level that is statistically significant, in the metabolic profile of samples from subjects with high stage kidney cancer as compared to another group (e.g., subjects not diagnosed with high stage kidney cancer) were identified as biomarkers to distinguish those groups.

The biomarkers are discussed in more detail herein. The biomarkers that were discovered correspond with biomarkers for distinguishing subjects having high stage kidney cancer vs. subjects having low stage kidney cancer (see Table 8).

Metabolic profiles were also determined for biological samples from human subjects diagnosed with more aggressive kidney cancer or human subjects diagnosed with less aggressive kidney cancer. The metabolic profile for biological samples from subjects having more aggressive kidney cancer were compared to the metabolic profile for biological samples from subjects having less aggressive kidney cancer. Those small molecules differentially present, including those small molecules differentially present at a level that is statistically significant, in the metabolic profile of samples from subjects with more aggressive kidney cancer as compared to another group (e.g., subjects not diagnosed with more aggressive kidney cancer) were identified as biomarkers to distinguish those groups.

The biomarkers are discussed in more detail herein. The biomarkers that were discovered correspond with biomarkers for distinguishing subjects having more aggressive kidney cancer vs. subjects having less aggressive kidney cancer (see Table 10).

### II. METHODS

A. Diagnosis of kidney cancer

The identification of biomarkers for kidney cancer allows for the diagnosis of (or for aiding in the diagnosis of) kidney cancer in subjects presenting with one or more symptoms consistent with the presence of kidney cancer and includes the initial diagnosis of kidney cancer in a subject not previously identified as having kidney cancer and diagnosis of recurrence of kidney cancer in a subject previously treated for kidney cancer. For example, an SRM may be detected in a subject during a medical examination making it necessary to determine if the SRM is cancer-positive or cancer-negative. A method of diagnosing (or aiding in diagnosing) whether a subject has kidney cancer comprises (1) analyzing a biological sample from a subject to determine the level(s) of one or more biomarkers of kidney cancer in the sample and (2) comparing the level(s) of the one or more biomarkers in the sample to kidney cancer-positive and/or kidney cancer-negative reference levels of the one or more biomarkers in order to diagnose (or aid in the diagnosis of) whether the subject has kidney cancer. The one or more biomarkers that are used are selected from Tables 1, 2, 4, and/or 11 and combinations thereof. When such a method is used to aid in the diagnosis of kidney cancer, the results of the method may be used along with other methods (or the results thereof) useful in the clinical determination of whether a subject has kidney cancer.

Any suitable method may be used to analyze the biological sample in order to determine the level(s) of the one or more biomarkers in the sample. Suitable methods include chromatography (e.g., HPLC, gas chromatography, liquid chromatography), mass spectrometry (e.g., MS, MS-MS), enzyme-linked immunosorbent assay (ELISA), antibody linkage, other immunochemical techniques, and combinations thereof. Further, the level(s) of the one or more biomarkers may be measured indirectly, for example, by using an assay that measures the level of a compound (or compounds) that correlates with the level of the biomarker(s) that are desired to be measured.

The levels of one or more of the biomarkers of Tables 1, 2, 4, and/or 11 may be determined in the methods of diagnosing and methods of aiding in diagnosing whether a subject has kidney cancer. For example, one or more of the following biomarkers may be used alone or in combination to diagnose or aid in diagnosing kidney cancer: oxidized glutathione (GSSG), proline, 2-oleoylglycerophosphoethanolamine, 2-aminobutyrate, sphingosine, 3-dehydrocarnitine, 2-docosahexaenoylglycerophosphocholine, 2-linoleoylglycerophosphocholine, phosphoethanolamine, glutamate, pyrophosphate (PPi), nicotinamide-adenine-dinucleotide (NAD+), 3-aminoisobutyrate, 2-arachidonoylglycerophosphoethanolamine, 2-arachidonoylglycerophosphocholine, 2-oleoylglycerophosphocholine, glycerate, choline-phosphate, pyruvate, 1-arachidonoylglycerophosphoethanolamine, adenine, 1-2-propanediol, 2-docosahexaenoylglycerophosphoethanolamine, 2-hydroxybutyrate (AHB), creatine, glycolate (hydroxyacetate), malate, 5-methylthioadenosine (MTA), stearolycarnitine, 1-arachidonoylglycerophosphoinositol, arachidonate, mannose-6-phosphate, alpha-tocopherol, flavin adenine dinucleotide (FAD), fructose-6-phosphate, maltose, maltotriose, fructose 1-phosphate, maltotetraose, 1-stearoylglycerophosphoinositol, methyl-alpha-glucopyranoside, glucose-6-phosphate (G6P), eicosenoate, 1-stearoylglycerophosphoethanolamine, 1-palmitoylglycerophosphoinositol, 1-oleoylglycerophosphoethanolamine, 1-palmitoylglycerophosphoethanolamine, 2-palmitoylglycerophosphoethanolamine, 1-oleoylglycerophosphoinositol, gamma-glutamylglutamate, ergothioneine, arabitol, 1-palmitoylplasmenylethanolamine, N-acetylneuraminate, malonylcarnitine, 2-hydroxyglutarate, beta-alanine, pantothenate, citrate, kynurenine, N1-methyladenosine, hippurate, glucose, N-acetylaspartate (NAA), N1-methylguanosine, pseudouridine, phenylacetylglutamine, N2-methylguanosine, 2-methylbutyrylcarnitine (C5), N-acetyl-aspartyl-glutamate (NAAG), N6-acetyllysine, dimethylarginine (SDMA+ADMA), methyl-4-hydroxybenzoate, catechol-sulfate, glycerol, 2-hydroxyhippurate (salicylurate), N(2)-furoyl-glycine, 3-hydroxyphenylacetate, gulono 1,4-lactone, 2-isopropylmalate, 2-3-dihydroxyisovalerate, 1-2-propanediol, gluconate, cinnamoylglycine, phenylacetylglycine, sorbose, sucrose, adenosine 5′-monophosphate (AMP), hexanoylglycine, methyl-indole-3-acetate, 3-hydroxyhippurate, N6-methyladenosine, 4-hydroxy-2-oxoglutaric acid, alpha-CEHC-glucuronide, phenylpropinylglycine, vanillate, ethanolamine, galactose, adipate, 2-oxindole-3-acetate, 1, 3-7-trimethylurate, and 3-4-dihydroxyphenylacetate. Additionally, for example, the level(s) of one biomarker, two or more biomarkers, three or more biomarkers, four or more biomarkers, five or more biomarkers, six or more biomarkers, seven or more biomarkers, eight or more biomarkers, nine or more biomarkers, ten or more biomarkers, etc., including a combination of all of the biomarkers in Tables 1, 2, 4, and/or 11 and combinations thereof or any fraction thereof, may be determined and used in such methods. Determining levels of combinations of the biomarkers may allow greater sensitivity and specificity in diagnosing kidney cancer and aiding in the diagnosis of kidney cancer. For example, ratios of the levels of certain biomarkers (and non-biomarker compounds) in biological samples may allow greater sensitivity and specificity in diagnosing kidney cancer and aiding in the diagnosis of kidney cancer.

After the level(s) of the one or more biomarkers in the sample are determined, the level(s) are compared to kidney cancer-positive and/or kidney cancer-negative reference levels to aid in diagnosing or to diagnose whether the subject has kidney cancer. Levels of the one or more biomarkers in a sample matching the kidney cancer-positive reference levels (e.g., levels that are the same as the reference levels, substantially the same as the reference levels, above and/or below the minimum and/or maximum of the reference levels, and/or within the range of the reference levels) are indicative of a diagnosis of kidney cancer in the subject. Levels of the one or more biomarkers in a sample matching the kidney cancer-negative reference levels (e.g., levels that are the same as the reference levels, substantially the same as the reference levels, above and/or below the minimum and/or maximum of the reference levels, and/or within the range of the reference levels) are indicative of a diagnosis of no kidney cancer in the subject. In addition, levels of the one or more biomarkers that are differentially present (especially at a level that is statistically significant) in the sample as compared to kidney cancer-negative reference levels are indicative of a diagnosis of kidney cancer in the subject. Levels of the one or more biomarkers that are differentially present (especially at a level that is statistically significant) in the sample as compared to kidney cancer-positive reference levels are indicative of a diagnosis of no kidney cancer in the subject.

The level(s) of the one or more biomarkers may be compared to kidney cancer-positive and/or kidney cancer-negative reference levels using various techniques, including a simple comparison (e.g., a manual comparison) of the level(s) of the one or more biomarkers in the biological sample to kidney cancer-positive and/or kidney cancer-negative reference levels. The level(s) of the one or more biomarkers in the biological sample may also be compared to kidney cancer-positive and/or kidney cancer-negative reference levels using one or more statistical analyses (e.g., t-test, Welch's T-test, Wilcoxon's rank sum test, Random Forest, T-score, Z-score) or using a mathematical model (e.g., algorithm, statistical model).

For example, a mathematical model comprising a single algorithm or multiple algorithms may be used to determine whether a subject has kidney cancer. A mathematical model may also be used to distinguish between kidney cancer stages. An exemplary mathematical model may use the measured levels of any number of biomarkers (for example, 2, 3, 5, 7, 9, etc.) from a subject to determine, using an algorithm or a series of algorithms based on mathematical relationships between the levels of the measured biomarkers, whether a subject has kidney cancer, whether kidney cancer is progressing or regressing in a subject, whether a subject has high stage or low stage kidney cancer, whether a subject has more aggressive or less aggressive kidney cancer, etc.

The results of the method may be used along with other methods (or the results thereof) useful in the diagnosis of kidney cancer in a subject.

In one aspect, the biomarkers provided herein can be used to provide a physician with an RCC Score indicating the existence and/or severity of kidney cancer in a subject. The score is based upon clinically significantly changed reference level(s) for a biomarker and/or combination of biomarkers. The reference level can be derived from an algorithm. The RCC Score can be used to place the subject in a severity range of kidney cancer from normal (i.e. no kidney cancer) to high. The RCC Score can be used in multiple ways: for example, disease progression, regression, or remission can be monitored by periodic determination and monitoring of the RCC Score; response to therapeutic intervention can be determined by monitoring the RCC Score; and drug efficacy can be evaluated using the RCC Score.

Methods for determining a subject's RCC Score may be performed using one or more of the kidney cancer biomarkers identified in Tables 1, 2, 4 and/or 11 in a biological sample. The method may comprise comparing the level(s) of the one or more kidney cancer biomarkers in the sample to kidney cancer reference levels of the one or more biomarkers in order to determine the subject's RCC score. The method may employ any number of markers selected from those listed in Table 1, 2, 4 and/or 11, including 1, 2, 3, 4, 5, 6, 7, 8, 9, 10, or more markers. Multiple biomarkers may be correlated with kidney cancer, by any method, including statistical methods such as regression analysis.

After the level(s) of the one or more biomarker(s) is determined, the level(s) may be compared to kidney cancer reference level(s) or reference curves of the one or more biomarker(s) to determine a rating for each of the one or more biomarker(s) in the sample. The rating(s) may be aggregated using any algorithm to create a score, for example, an RCC score, for the subject. The algorithm may take into account any factors relating to kidney cancer including the number of biomarkers, the correlation of the biomarkers to kidney cancer, etc.

In an embodiment, a mathematical model or formula containing one or more biomarkers as variables is established using regression analysis, e.g., multiple linear regressions. By way of non-limiting example, the developed formulas may include the following:

A+B(Biomarker₁)+C(Biomarker₂)+D(Biomarker₃)+E(Biomarker₄)=RScore

A+B*ln(Biomarker₁)+C*ln(Biomarker₂)+D*ln(Biomarker₃)+E*ln(Biomarker₄)=ln RScore

wherein A, B, C, D, E are constant numbers; Biomarker₁, Biomarker₂, Biomarker₃, Biomarker₄ are the measured values of the analyte (Biomarker) and RScore is the measure of cancer presence or absence or cancer aggressivity.

The formulas may include one or more biomarkers as variables, such as 1, 2, 3, 4, 5, 10, 15, 20 or more biomarkers.

Additionally, in one embodiment, the biomarkers provided herein to diagnose or aid in the diagnosis of kidney cancer may be used to distinguish kidney cancer from other urological cancers. A method of distinguishing kidney cancer from other urological cancers in a subject comprises (1) analyzing a biological sample from a subject to determine the level(s) of one or more biomarkers of kidney cancer in the sample and (2) comparing the level(s) of the one or more biomarkers in the sample to kidney cancer-positive and/or kidney cancer-negative reference levels of the one or more biomarkers in order to distinguish kidney cancer from other urological cancers. The one or more biomarkers that are used are selected from Table 11. For example, one or more of the following biomarkers may be used alone or in any combination to distinguish kidney cancer from other urological cancers: gluconate, 1,2-propanediol, galactose, gulono 1,4-lactone, orotidine, quinate, 1, 3-7-trimethylurate, guanine, phenylacetylglutamine, mannitol, 2-oxindole-3-acetate, 1,3-aminopropyl-2-pyrrolidone, 1,3-dimethylurate, glucuronate-galacturonate-5-keto-gluconate, glycocholate, azelate (nonanedioate), N-acetylthreonine, 7-ketodeoxycholate, 3-sialyllactose, isovalerylcarnitine, cholate, adenosine 5′ monophosphate (AMP), 2-hydroxyisobutyrate, 4-hydroxyhippurate, pipecolate, N-acetylphenylalanine, 12-dehydrocholate, alpha-ketoglutarate, sulforaphane, 3-indoxyl-sulfate, methyl-indole-3-acetate, methyl-4-hydroxybenzoate, lactate, N(2)-furoyl-glycine, N6-methyladenosine, gamma-CEHC, glycerol, 2-3-butanediol, palmitoyl-sphingomyelin, succinate, 4-hydroxyphenylacetate, caffeate, imidazole-pripionate, beta-alanine, 4-androsten-3beta-17beta-diol-disulfate-2,5-methylthioadenosine (MTA), N2-acetyllysine, sucrose, phenylacetylglycine, 4-androsten-3beta-17beta-diol-disulfate-1, cyclo-gly-pro, N-methyl-proline, catechol-sulfate, serine, vanillate, threonine, and 21-hydroxypregnenolone-disulfate. When such a method is used to distinguish kidney cancer from other urological cancers, the results of the method may be used along with other methods (or the results thereof) useful in the clinical determination of distinguishing kidney cancer from other urological cancers.

B. Methods of Monitoring Progression/Regression of Kidney Cancer

The identification of biomarkers for kidney cancer also allows for monitoring progression/regression of kidney cancer in a subject. A method of monitoring the progression/regression of kidney cancer in a subject comprises (1) analyzing a first biological sample from a subject to determine the level(s) of one or more biomarkers for kidney cancer selected from Tables 1, 2, 4, 8, 10 and/or 11, the first sample obtained from the subject at a first time point, (2) analyzing a second biological sample from a subject to determine the level(s) of the one or more biomarkers, the second sample obtained from the subject at a second time point, and (3) comparing the level(s) of one or more biomarkers in the first sample to the level(s) of the one or more biomarkers in the second sample in order to monitor the progression/regression of kidney cancer in the subject. The results of the method are indicative of the course of kidney cancer (i.e., progression or regression, if any change) in the subject.

The levels of one or more of the biomarkers of Tables 1, 2, 4, 8, 10 and/or 11 may be determined in the methods of monitoring progression/regression of kidney cancer. For example, one or more of the following biomarkers may be used alone or in combination to monitor the progression/regression of kidney cancer: oxidized glutathione (GSSG), proline, 2-oleoylglycerophosphoethanolamine, 2-aminobutyrate, sphingosine, 3-dehydrocamitine, 2-docosahexaenoylglycerophosphocholine, 2-linoleoylglycerophosphocholine, phosphoethanolamine, glutamate, pyrophosphate (PPi), nicotinamide-adenine-dinucleotide (NAD+), 3-aminoisobutyrate, 2-arachidonoylglycerophosphoethanolamine, 2-arachidonoylglycerophosphocholine, 2-oleoylglycerophosphocholine, glycerate, choline-phosphate, pyruvate, 1-arachidonoylglycerophosphoethanolamine, adenine, 1-2-propanediol, 2-docosahexaenoylglycerophosphoethanolamine, 2-hydroxybutyrate (AHB), creatine, glycolate (hydroxyacetate), malate, 5-methylthioadenosine (MTA), stearolycamitine, 1-arachidonoylglycerophosphoinositol, arachidonate, mannose-6-phosphate, alpha-tocopherol, flavin adenine dinucleotide (FAD), fructose-6-phosphate, maltose, maltotriose, fructose 1-phosphate, maltotetraose, 1-stearoylglycerophosphoinositol, methyl-alpha-glucopyranoside, glucose-6-phosphate (G6P), eicosenoate, 1-stearoylglycerophosphoethanolamine, 1-palmitoylglycerophosphoinositol, 1-oleoylglycerophosphoethanolamine, 1-palmitoylglycerophosphoethanolamine, 2-palmitoylglycerophosphoethanolamine, 1-oleoylglycerophosphoinositol, gamma-glutamylglutamate, ergothioneine, arabitol, 1-palmitoylplasmenylethanolamine, N-acetylneuraminate, malonylcarnitine, 2-hydroxyglutarate, beta-alanine, pantothenate, citrate, kynurenine, N1-methyladenosine, hippurate, glucose, N-acetylaspartate (NAA), N1-methylguanosine, pseudouridine, phenylacetylglutamine, N2-methylguanosine, 2-methylbutyrylcarnitine (C5), N-acetyl-aspartyl-glutamate (NAAG), N6-acetyllysine, dimethylarginine (SDMA+ADMA), methyl-4-hydroxybenzoate, catechol-sulfate, glycerol, 2-hydroxyhippurate (salicylurate), N(2)-furoyl-glycine, 3-hydroxyphenylacetate, gulono 1,4-lactone, 2-isopropylmalate, 2-3-dihydroxyisovalerate, 1-2-propanediol, gluconate, cinnamoylglycine, phenylacetylglycine, sorbose, sucrose, adenosine 5′-monophosphate (AMP), hexanoylglycine, methyl-indole-3-acetate, 3-hydroxyhippurate, N6-methyladenosine, 4-hydroxy-2-oxoglutaric acid, alpha-CEHC-glucuronide, phenylpropinylglycine, vanillate, ethanolamine, galactose, adipate, 2-oxindole-3-acetate, 1, 3-7-trimethylurate, 3-4-dihydroxyphenylacetate, choline, pelargonate (9:0), arginine, gamma-glutamylleucine, xanthine, tyrosine, 5-oxoproline, inositiol-1-phosphate (I1P), isoleucine, 2-ethylhexanoate, leucine, laurate (12:0), phenylalanine, mannose, uracil, xanthosine, erythritol, guanosine-5-monophosphate-5 (GMP), homocysteine, lactate, 4-hydroxybutyrate (GHB), ribose, fucose, S-adenosylhomocysteine (SAH), mannitol, hypoxanthine, and threonine. Additionally, for example, the level(s) of one biomarker, two or more biomarkers, three or more biomarkers, four or more biomarkers, five or more biomarkers, six or more biomarkers, seven or more biomarkers, eight or more biomarkers, nine or more biomarkers, ten or more biomarkers, etc., including a combination of all of the biomarkers in Tables 1, 2, 4, 8, 10 and 11 or any fraction thereof, may be determined and used in methods of monitoring the progression/regression of kidney cancer in a subject.

The change (if any) in the level(s) of the one or more biomarkers over time may be indicative of progression or regression of kidney cancer in the subject. In order to characterize the course of kidney cancer in the subject, the level(s) of the one or more biomarkers in the first sample, the level(s) of the one or more biomarkers in the second sample, and/or the results of the comparison of the levels of the biomarkers in the first and second samples may be compared to kidney cancer-positive and kidney cancer-negative reference levels. If the comparisons indicate that the level(s) of the one or more biomarkers are increasing or decreasing over time (e.g., in the second sample as compared to the first sample) to become more similar to the kidney cancer-positive reference levels (or less similar to the kidney cancer-negative reference levels), then the results are indicative of kidney cancer progression. If the comparisons indicate that the level(s) of the one or more biomarkers are increasing or decreasing over time to become more similar to the kidney cancer-negative reference levels (or less similar to the kidney cancer-positive reference levels), then the results are indicative of kidney cancer regression.

In one embodiment, the assessment may be based on an RCC Score which is indicative of kidney cancer in the subject and which can be monitored over time. By comparing the RCC Score from a first time point sample to the RCC Score from at least a second time point sample the progression or regression of kidney cancer can be determined. Such a method of monitoring the progression/regression of kidney cancer in a subject comprises (1) analyzing a first biological sample from a subject to determine an RCC score for the first sample obtained from the subject at a first time point, (2) analyzing a second biological sample from a subject to determine a second RCC score, the second sample obtained from the subject at a second time point, and (3) comparing the RCC score in the first sample to the RCC score in the second sample in order to monitor the progression/regression of kidney cancer in the subject.

The biomarkers and algorithms described herein may guide or assist a physician in deciding a treatment path, for example, whether to implement procedures such as surgical procedures (e.g., full or partial nephrectomy), treat with drug therapy, or employ a watchful waiting approach.

As with the other methods described herein, the comparisons made in the methods of monitoring progression/regression of kidney cancer in a subject may be carried out using various techniques, including simple comparisons, one or more statistical analyses, mathematical models (algorithms) and combinations thereof.

The results of the method may be used along with other methods (or the results thereof) useful in the clinical monitoring of progression/regression of kidney cancer in a subject.

As described above in connection with methods of diagnosing (or aiding in the diagnosis of) kidney cancer, any suitable method may be used to analyze the biological samples in order to determine the level(s) of the one or more biomarkers in the samples. In addition, the level(s) one or more biomarkers, including a combination of all of the biomarkers in Tables 1, 2, 4, 8, 10 and/or 11 or any fraction thereof, may be determined and used in methods of monitoring progression/regression of kidney cancer in a subject.

Such methods could be conducted to monitor the course of kidney cancer in subjects having kidney cancer or could be used in subjects not having kidney cancer (e.g., subjects suspected of being predisposed to developing kidney cancer) in order to monitor levels of predisposition to kidney cancer.

C. Methods of Staging Kidney Cancer

The identification of biomarkers for kidney cancer also allows for the determination of kidney cancer stage of a subject, including the cancer stage of a subject having a cancer-positive SRM. A method of determining the stage of kidney cancer comprises (1) analyzing a biological sample from a subject to determine the level(s) of one or more biomarkers listed in Table 8 in the sample and (2) comparing the level(s) of the one or more biomarkers in the sample to high stage kidney cancer and/or low stage kidney cancer reference levels of the one or more biomarkers in order to determine the stage of the subject's kidney cancer. The results of the method may be used along with other methods (or the results thereof) useful in the clinical determination of the stage of a subject's kidney cancer.

As described above in connection with methods of diagnosing (or aiding in the diagnosis of) kidney cancer, any suitable method may be used to analyze the biological sample in order to determine the level(s) of the one or more biomarkers in the sample.

The levels of one or more biomarkers listed in Table 8 and combinations thereof may be determined in the methods of determining the stage of a subject's kidney cancer. For example, one or more of the following biomarkers may be used alone or in combination to determine the stage of kidney cancer: choline, pelargonate (9:0), arginine, gamma-glutamylleucine, xanthine, tyrosine, 5-oxoproline, inositiol-1-phosphate (HP), N2-methylguanosine, isoleucine, 2-ethylhexanoate, leucine, adenine, 5-methylthioadenosine (MTA), laurate (12:0), phenylalanine, mannose, uracil, xanthosine, erythritol, guanosine-5-monophosphate-5 (GMP), homocysteine, lactate, 4-hydroxybutyrate (GHB), ribose, fucose, S-adenosylhomocysteine (SAH), mannitol, hypoxanthine, and threonine. Additionally, for example, the level(s) of one biomarker, two or more biomarkers, three or more biomarkers, four or more biomarkers, five or more biomarkers, six or more biomarkers, seven or more biomarkers, eight or more biomarkers, nine or more biomarkers, ten or more biomarkers, etc., including a combination of all of the biomarkers in Table 8 or any fraction thereof, may be determined and used in methods of determining the stage of kidney cancer of a subject.

After the level(s) of the one or more biomarkers in a sample are determined, the level(s) are compared to low stage kidney cancer and/or high stage kidney cancer reference levels in order to predict the stage of kidney cancer of a subject. Levels of the one or more biomarkers in a sample matching the high stage kidney cancer reference levels (e.g., levels that are the same as the reference levels, substantially the same as the reference levels, above and/or below the minimum and/or maximum of the reference levels, and/or within the range of the reference levels) are indicative of the subject having high stage kidney cancer. Levels of the one or more biomarkers in a sample matching the low stage kidney cancer reference levels (e.g., levels that are the same as the reference levels, substantially the same as the reference levels, above and/or below the minimum and/or maximum of the reference levels, and/or within the range of the reference levels) are indicative of the subject having low stage kidney cancer. In addition, levels of the one or more biomarkers that are differentially present (especially at a level that is statistically significant) in the sample as compared to low stage kidney cancer reference levels are indicative of the subject not having low stage kidney cancer. Levels of the one or more biomarkers that are differentially present (especially at a level that is statistically significant) in the sample as compared to high stage kidney cancer reference levels are indicative of the subject not having high stage kidney cancer.

Studies were carried out to identify a set of biomarkers that can be used to determine the kidney cancer stage of a subject. In another embodiment, the biomarkers provided herein can be used to provide a physician with an RCC Score indicating the stage of kidney cancer in a subject. The score is based upon clinically significantly changed reference level(s) for a biomarker and/or combination of biomarkers. The reference level can be derived from an algorithm. The RCC Score can be used to determine the stage of kidney cancer in a subject from normal (i.e. no kidney cancer) to high stage kidney cancer.

The biomarkers and algorithms described herein may guide or assist a physician in deciding a treatment path, for example, whether to implement procedures such as surgical procedures (e.g., full or partial nephrectomy), treat with drug therapy, or employ a watchful waiting approach.

As with the methods described above, the level(s) of the one or more biomarkers may be compared to high stage kidney cancer and/or low stage kidney cancer reference levels using various techniques, including a simple comparison, one or more statistical analyses, and combinations thereof.

As with the methods of diagnosing (or aiding in diagnosing) whether a subject has kidney cancer, the methods of determining the stage of kidney cancer of a subject may further comprise analyzing the biological sample to determine the level(s) of one or more non-biomarker compounds.

D. Methods of Distinguishing Less Aggressive Kidney Cancer from More aggressive Kidney Cancer

The identification of biomarkers for kidney cancer also allows for the identification of biomarkers for distinguishing less aggressive kidney cancer from more aggressive kidney cancer, including distinguishing less aggressive cancer-positive SRMs from more aggressive cancer-positive SRMs. A method of distinguishing less aggressive kidney cancer from more aggressive kidney cancer in a subject having kidney cancer comprises (1) analyzing a biological sample from a subject to determine the level(s) of one or more biomarkers listed in Table 10 in the sample and (2) comparing the level(s) of the one or more biomarkers in the sample to less aggressive kidney cancer and/or more aggressive kidney cancer reference levels of the one or more biomarkers in order to determine the aggressiveness of the subject's kidney cancer. The results of the method may be used along with other methods (or the results thereof) useful in the clinical determination of the aggressiveness of a subject's kidney cancer.

As described above in connection with methods of diagnosing (or aiding in the diagnosis of) kidney cancer, any suitable method may be used to analyze the biological sample in order to determine the level(s) of the one or more biomarkers in the sample.

The levels of one or more biomarkers listed in Tables 4 and/or 10 may be determined in the methods of determining the aggressiveness of a subject's kidney cancer. For example, one or more of the following biomarkers may be used alone or in combination to determine the aggressiveness of a subject's kidney cancer:pelargonate (9:0), laurate (12:0), homocysteine, 2′-deoxyinosine, S-adenosylmethionine (SAM), glycylthreonine, aspartylphenylalanine, phenylalanylglycine, cytidine 5′-diphosphocholine, alanylglycine, lysylmethionine, glycylisoleucine, ribose, aspartylleucine, 2-ethylhexanoate, asparagine, homoserine, 2′-deoxyguanosine, valerylcarnitine, 4-hydroxybutyrate (GHB), caprate (10:0), galactose, heme, butyrylcarnitine, choline, isoleucine, mannitol, fucose, tyrosine, xanthine, 5-oxoproline, 5-methylthioadenosine (MTA), phenylalanine, leucine, threonate, gamma-glutamylleucine, benzoate, proline, methionine, glycylproline, N2-methylguanosine, adenine, 2-methylbutyroylcarnitine, S-adenosylhomocysteine (SAH), citrate, xanthosine, 5,6-dihydrouracil, threonine, valine, and pantothenate. Additionally, for example, as with the methods of diagnosing (or aiding in the diagnosis of) kidney cancer described above, the level(s) of one biomarker, two or more biomarkers, three or more biomarkers, four or more biomarkers, five or more biomarkers, six or more biomarkers, seven or more biomarkers, eight or more biomarkers, nine or more biomarkers, ten or more biomarkers, etc., including a combination of all of the biomarkers in Tables 4 and 10 or any fraction thereof, may be determined and used in methods of determining the aggressiveness of kidney cancer of a subject.

After the level(s) of the one or more biomarkers in the sample are determined, the level(s) are compared to less aggressive kidney cancer and/or more aggressive kidney cancer reference levels in order to determine the aggressiveness of kidney cancer of a subject. Levels of the one or more biomarkers in a sample matching the more aggressive kidney cancer reference levels (e.g., levels that are the same as the reference levels, substantially the same as the reference levels, above and/or below the minimum and/or maximum of the reference levels, and/or within the range of the reference levels) are indicative of the subject having more aggressive kidney cancer. Levels of the one or more biomarkers in a sample matching the less aggressive kidney cancer reference levels (e.g., levels that are the same as the reference levels, substantially the same as the reference levels, above and/or below the minimum and/or maximum of the reference levels, and/or within the range of the reference levels) are indicative of the subject having less aggressive kidney cancer. In addition, levels of the one or more biomarkers that are differentially present (especially at a level that is statistically significant) in the sample as compared to less aggressive kidney cancer reference levels are indicative of the subject not having less aggressive kidney cancer. Levels of the one or more biomarkers that are differentially present (especially at a level that is statistically significant) in the sample as compared to more aggressive kidney cancer reference levels are indicative of the subject not having more aggressive kidney cancer.

Studies were carried out to identify a set of biomarkers that can be used to distinguish less aggressive kidney cancer from more aggressive kidney cancer. In another embodiment, the biomarkers provided herein can be used to provide a physician with an RCC Score indicating the aggressiveness of kidney cancer in a subject. The score is based upon clinically significantly changed reference level(s) for a biomarker and/or combination of biomarkers. The reference level can be derived from an algorithm. The RCC Score can be used to determine the aggressiveness of kidney cancer in a subject from normal (i.e. no kidney cancer) to more aggressive kidney cancer.

The biomarkers and algorithms described herein may guide or assist a physician in deciding a treatment path, for example, whether to implement procedures such as surgical procedures (e.g., full or partial nephrectomy), treat with drug therapy, or employ a watchful waiting approach.

As with the methods described above, the level(s) of the one or more biomarkers may be compared to more aggressive kidney cancer and/or less aggressive kidney cancer reference levels using various techniques, including a simple comparison, one or more statistical analyses, and combinations thereof.

As with the methods of diagnosing (or aiding in diagnosing) whether a subject has kidney cancer, the methods of determining the aggressiveness of kidney cancer of a subject may further comprise analyzing the biological sample to determine the level(s) of one or more non-biomarker compounds.

E. Methods of Determining Whether a Small Renal Mass (SRM) is Cancerous

The identification of biomarkers for kidney cancer also allows for the determination of whether a subject discovered as having an SRM has a benign SRM or an SRM that is cancerous. A method of determining the cancer status of an SRM comprises (1) analyzing a biological sample from a subject to determine the level(s) of one or more biomarkers listed in Tables 1, 2, 4, 8, 10, and/or 11 in the sample and (2) comparing the level(s) of the one or more biomarkers in the sample to kidney cancer-positive and/or kidney cancer-negative reference levels of the one or more biomarkers in order to determine the cancer status of the subject's SRM. The results of the method may be used along with other methods (or the results thereof) useful in the clinical determination of the cancer status of a subject's SRM.

As described above in connection with methods of diagnosing (or aiding in the diagnosis of) kidney cancer, any suitable method may be used to analyze the biological sample in order to determine the level(s) of the one or more biomarkers in the sample.

As with the methods of diagnosing (or aiding in the diagnosis of) kidney cancer described above, the level(s) of one or more of the biomarkers in Tables 1, 2, 4, 8, 10 and/or 11 may be determined in the methods of determining the cancer status of an SRM. For example, one or more of the following biomarkers may be used alone or in combination to determine the cancer status of a subject's SRM: oxidized glutathione (GSSG), proline, 2-oleoylglycerophosphoethanolamine, 2-aminobutyrate, sphingosine, 3-dehydrocarnitine, 2-docosahexaenoylglycerophosphocholine, 2-linoleoylglycerophosphocholine, phosphoethanolamine, glutamate, pyrophosphate (PPi), nicotinamide-adenine-dinucleotide (NAD+), 3-aminoisobutyrate, 2-arachidonoylglycerophosphoethanolamine, 2-arachidonoylglycerophosphocholine, 2-oleoylglycerophosphocholine, glycerate, choline-phosphate, pyruvate, 1-arachidonoylglycerophosphoethanolamine, adenine, 1-2-propanediol, 2-docosahexaenoylglycerophosphoethanolamine, 2-hydroxybutyrate (AHB), creatine, glycolate (hydroxyacetate), malate, 5-methylthioadenosine (MTA), stearolycamitine, 1-arachidonoylglycerophosphoinositol, arachidonate, mannose-6-phosphate, alpha-tocopherol, flavin adenine dinucleotide (FAD), fructose-6-phosphate, maltose, maltotriose, fructose 1-phosphate, maltotetraose, 1-stearoylglycerophosphoinositol, methyl-alpha-glucopyranoside, glucose-6-phosphate (G6P), eicosenoate, 1-stearoylglycerophosphoethanolamine, 1-palmitoylglycerophosphoinositol, 1-ol eoylglycerophosphoethanolamine, 1-palmitoylglycerophosphoethanolamine, 2-palmitoylglycerophosphoethanolamine, 1-oleoylglycerophosphoinositol, gamma-glutamylglutamate, ergothioneine, arabitol, 1-palmitoylplasmenylethanolamine, N-acetylneuraminate, malonylcarnitine, 2-hydroxyglutarate, beta-alanine, pantothenate, citrate, kynurenine, N1-methyladenosine, hippurate, glucose, N-acetylaspartate (NAA), N1-methylguanosine, pseudouridine, phenylacetylglutamine, N2-methylguanosine, 2-methylbutyrylcarnitine (C5), N-acetyl-aspartyl-glutamate (NAAG), N6-acetyllysine, dimethylarginine (SDMA+ADMA), methyl-4-hydroxybenzoate, catechol-sulfate, glycerol, 2-hydroxyhippurate (salicylurate), N(2)-furoyl-glycine, 3-hydroxyphenylacetate, gulono 1,4-lactone, 2-isopropylmalate, 2-3-dihydroxyisovalerate, 1-2-propanediol, gluconate, cinnamoylglycine, phenylacetylglycine, sorbose, sucrose, adenosine 5′-monophosphate (AMP), hexanoylglycine, methyl-indole-3-acetate, 3-hydroxyhippurate, N6-methyladenosine, 4-hydroxy-2-oxoglutaric acid, alpha-CEHC-glucuronide, phenylpropinylglycine, vanillate, ethanolamine, galactose, adipate, 2-oxindole-3-acetate, 1, 3-7-trimethylurate, and 3-4-dihydroxyphenylacetate. Additionally, for example, the level(s) of one biomarker, two or more biomarkers, three or more biomarkers, four or more biomarkers, five or more biomarkers, six or more biomarkers, seven or more biomarkers, eight or more biomarkers, nine or more biomarkers, ten or more biomarkers, etc., including a combination of all of the biomarkers in Tables 1, 2, 4, 8, 10, and/or 11 or any fraction thereof, may be determined and used in methods of determining the cancer status of a subject's SRM.

After the level(s) of the one or more biomarkers in a sample are determined, the level(s) are compared to kidney cancer-positive and/or kidney cancer-negative reference levels in order to determine the cancer status of a subject's SRM. Levels of the one or more biomarkers in a sample matching the kidney cancer-positive reference levels (e.g., levels that are the same as the reference levels, substantially the same as the reference levels, above and/or below the minimum and/or maximum of the reference levels, and/or within the range of the reference levels) are indicative of the subject having a cancer-positive SRM. Levels of the one or more biomarkers in a sample matching the kidney cancer-negative reference levels (e.g., levels that are the same as the reference levels, substantially the same as the reference levels, above and/or below the minimum and/or maximum of the reference levels, and/or within the range of the reference levels) are indicative of the subject having a cancer-negative SRM. In addition, levels of the one or more biomarkers that are differentially present (especially at a level that is statistically significant) in the sample as compared to kidney cancer-negative reference levels are indicative of a diagnosis of a cancer-positive SRM. Levels of the one or more biomarkers that are differentially present (especially at a level that is statistically significant) in the sample as compared to kidney cancer-positive reference levels are indicative of the subject not having a cancer-positive SRM.

As with the methods described above, the level(s) of the one or more biomarkers may be compared to kidney cancer-positive and/or kidney cancer-negative reference levels using various techniques, including a simple comparison, one or more statistical analyses, and combinations thereof. An RCC Score may also be used in indicating the existence and/or severity of cancer in a SRM.

As with the methods of diagnosing (or aiding in diagnosing) whether a subject has kidney cancer, the methods of assessing the cancer status of a SRM of a subject may further comprise analyzing the biological sample to determine the level(s) of one or more non-biomarker compounds.

F. Methods of Assessing Efficacy of Compositions for Treating Kidney Cancer

The identification of biomarkers for kidney cancer also allows for assessment of the efficacy of a composition for treating kidney cancer as well as the assessment of the relative efficacy of two or more compositions for treating kidney cancer. Such assessments may be used, for example, in efficacy studies as well as in lead selection of compositions for treating kidney cancer.

A method of assessing the efficacy of a composition for treating kidney cancer comprises (1) analyzing, from a subject having kidney cancer and currently or previously being treated with a composition, a biological sample to determine the level(s) of one or more biomarkers selected from Tables 1, 2, 4, 8, 10 and/or 11, and (2) comparing the level(s) of the one or more biomarkers in the sample to (a) level(s) of the one or more biomarkers in a previously-taken biological sample from the subject, wherein the previously-taken biological sample was obtained from the subject before being treated with the composition, (b) kidney cancer-positive reference levels of the one or more biomarkers, and (c) kidney cancer-negative reference levels of the one or more biomarkers. The results of the comparison are indicative of the efficacy of the composition for treating kidney cancer.

The levels of one or more of the biomarkers of Tables 1, 2, 4, 8, 10 and/or 11 may be determined in the methods of assessing the efficacy of a composition for treating kidney cancer. For example, one or more of the following biomarkers may be used alone or in combination to assess the efficacy of a composition for treating kidney cancer: oxidized glutathione (GSSG), proline, 2-oleoylglycerophosphoethanolamine, 2-aminobutyrate, sphingosine, 3-dehydrocarnitine, 2-docosahexaenoylglycerophosphocholine, 2-linoleoylglycerophosphocholine, phosphoethanolamine, glutamate, pyrophosphate (PPi), nicotinamide-adenine-dinucleotide (NAD+), 3-aminoisobutyrate, 2-arachidonoylglycerophosphoethanolamine, 2-arachidonoylglycerophosphocholine, 2-oleoylglycerophosphocholine, glycerate, choline-phosphate, pyruvate, 1-arachidonoylglycerophosphoethanolamine, adenine, 1-2-propanediol, 2-docosahexaenoylglycerophosphoethanolamine, 2-hydroxybutyrate (AHB), creatine, glycolate (hydroxyacetate), malate, 5-methylthioadenosine (MTA), stearolycarnitine, 1-arachidonoylglycerophosphoinositol, arachidonate, mannose-6-phosphate, alpha-tocopherol, flavin adenine dinucleotide (FAD), fructose-6-phosphate, maltose, maltotriose, fructose 1-phosphate, maltotetraose, 1-stearoylglycerophosphoinositol, methyl-alpha-glucopyranoside, glucose-6-phosphate (G6P), eicosenoate, 1-stearoylglycerophosphoethanolamine, 1-palmitoylglycerophosphoinositol, 1-oleoylglycerophosphoethanolamine, 1-palmitoylglycerophosphoethanolamine, 2-palmitoylglycerophosphoethanolamine, 1-oleoylglycerophosphoinositol, gamma-glutamylglutamate, ergothioneine, arabitol, 1-palmitoylplasmenylethanolamine, N-acetylneuraminate, malonylcarnitine, 2-hydroxyglutarate, beta-alanine, pantothenate, citrate, kynurenine, N1-methyladenosine, hippurate, glucose, N-acetylaspartate (NAA), N1-methylguanosine, pseudouridine, phenylacetylglutamine, N2-methylguanosine, 2-methylbutyrylcarnitine (C5), N-acetyl-aspartyl-glutamate (NAAG), N⁶-acetyllysine, dimethylarginine (SDMA+ADMA), methyl-4-hydroxybenzoate, catechol-sulfate, glycerol, 2-hydroxyhippurate (salicylurate), N(2)-furoyl-glycine, 3-hydroxyphenylacetate, gulono 1,4-lactone, 2-isopropylmalate, 2-3-dihydroxyisovalerate, 1-2-propanediol, gluconate, cinnamoylglycine, phenylacetylglycine, sorbose, sucrose, adenosine 5′-monophosphate (AMP), hexanoylglycine, methyl-indole-3-acetate, 3-hydroxyhippurate, N6-methyladenosine, 4-hydroxy-2-oxoglutaric acid, alpha-CEHC-glucuronide, phenylpropinylglycine, vanillate, ethanolamine, galactose, adipate, 2-oxindole-3-acetate, 1, 3-7-trimethylurate, 3-4-dihydroxyphenylacetate, choline, pelargonate (9:0), arginine, gamma-glutamylleucine, xanthine, tyrosine, 5-oxoproline, inositiol-1-phosphate (11P), isoleucine, 2-ethylhexanoate, leucine, laurate (12:0), phenylalanine, mannose, uracil, xanthosine, erythritol, guanosine-5-monophosphate-5 (GMP), homocysteine, lactate, 4-hydroxybutyrate (GHB), ribose, fucose, S-adenosylhomocysteine (SAH), mannitol, hypoxanthine, and threonine. Additionally, for example, the level(s) of one biomarker, two or more biomarkers, three or more biomarkers, four or more biomarkers, five or more biomarkers, six or more biomarkers, seven or more biomarkers, eight or more biomarkers, nine or more biomarkers, ten or more biomarkers, etc., including a combination of all of the biomarkers in Tables 1, 2, 4, 8, 10 and 11 or any fraction thereof, may be determined and used in methods of assessing the efficacy of a composition for treating kidney cancer.

Thus, in order to characterize the efficacy of the composition for treating kidney cancer, the level(s) of the one or more biomarkers in the biological sample are compared to (1) kidney cancer-positive reference levels, (2) kidney cancer-negative reference levels, and (3) previous levels of the one or more biomarkers in the subject before treatment with the composition.

When comparing the level(s) of the one or more biomarkers in the biological sample (from a subject having kidney cancer and currently or previously being treated with a composition) to kidney cancer-positive reference levels and/or kidney cancer-negative reference levels, level(s) in the sample matching the kidney cancer-negative reference levels (e.g., levels that are the same as the reference levels, substantially the same as the reference levels, above and/or below the minimum and/or maximum of the reference levels, and/or within the range of the reference levels) are indicative of the composition having efficacy for treating kidney cancer. Levels of the one or more biomarkers in the sample matching the kidney cancer-positive reference levels (e.g., levels that are the same as the reference levels, substantially the same as the reference levels, above and/or below the minimum and/or maximum of the reference levels, and/or within the range of the reference levels) are indicative of the composition not having efficacy for treating kidney cancer. The comparisons may also indicate degrees of efficacy for treating kidney cancer based on the level(s) of the one or more biomarkers.

When the level(s) of the one or more biomarkers in the biological sample (from a subject having kidney cancer and currently or previously being treated with a composition) are compared to level(s) of the one or more biomarkers in a previously-taken biological sample from the subject before treatment with the composition, any changes in the level(s) of the one or more biomarkers are indicative of the efficacy of the composition for treating kidney cancer. That is, if the comparisons indicate that the level(s) of the one or more biomarkers have increased or decreased after treatment with the composition to become more similar to the kidney cancer-negative reference levels (or less similar to the kidney cancer-positive reference levels), then the results are indicative of the composition having efficacy for treating kidney cancer. If the comparisons indicate that the level(s) of the one or more biomarkers have not increased or decreased after treatment with the composition to become more similar to the kidney cancer-negative reference levels (or less similar to the kidney cancer-positive reference levels), then the results are indicative of the composition not having efficacy for treating kidney cancer. The comparisons may also indicate degrees of efficacy for treating kidney cancer based on the amount of changes observed in the level(s) of the one or more biomarkers after treatment. In order to help characterize such a comparison, the changes in the level(s) of the one or more biomarkers, the level(s) of the one or more biomarkers before treatment, and/or the level(s) of the one or more biomarkers in the subject currently or previously being treated with the composition may be compared to kidney cancer-positive reference levels, and/or to kidney cancer-negative reference levels.

Another method for assessing the efficacy of a composition in treating kidney cancer comprises (1) analyzing a first biological sample from a subject to determine the level(s) of one or more biomarkers selected from Tables 1, 2, 4, 8, 10 and/or 11, the first sample obtained from the subject at a first time point, (2) administering the composition to the subject, (3) analyzing a second biological sample from a subject to determine the level(s) of the one or more biomarkers, the second sample obtained from the subject at a second time point after administration of the composition, and (4) comparing the level(s) of one or more biomarkers in the first sample to the level(s) of the one or more biomarkers in the second sample in order to assess the efficacy of the composition for treating kidney cancer. As indicated above, if the comparison of the samples indicates that the level(s) of the one or more biomarkers have increased or decreased after administration of the composition to become more similar to the kidney cancer-negative reference levels, then the results are indicative of the composition having efficacy for treating kidney cancer. If the comparisons indicate that the level(s) of the one or more biomarkers have not increased or decreased after treatment with the composition to become more similar to the kidney cancer-negative reference levels (or less similar to the kidney cancer-positive reference levels) then the results are indicative of the composition not having efficacy for treating kidney cancer. The comparison may also indicate a degree of efficacy for treating kidney cancer based on the amount of changes observed in the level(s) of the one or more biomarkers after administration of the composition as discussed above.

A method of assessing the relative efficacy of two or more compositions for treating kidney cancer comprises (1) analyzing, from a first subject having kidney cancer and currently or previously being treated with a first composition, a first biological sample to determine the level(s) of one or more biomarkers selected from Tables 1, 2, 4, 8, 10 and/or 11 (2) analyzing, from a second subject having kidney cancer and currently or previously being treated with a second composition, a second biological sample to determine the level(s) of the one or more biomarkers, and (3) comparing the level(s) of one or more biomarkers in the first sample to the level(s) of the one or more biomarkers in the second sample in order to assess the relative efficacy of the first and second compositions for treating kidney cancer. The results are indicative of the relative efficacy of the two compositions, and the results (or the levels of the one or more biomarkers in the first sample and/or the level(s) of the one or more biomarkers in the second sample) may be compared to kidney cancer-positive reference levels, kidney cancer-negative reference levels to aid in characterizing the relative efficacy.

Each of the methods of assessing efficacy may be conducted on one or more subjects or one or more groups of subjects (e.g., a first group being treated with a first composition and a second group being treated with a second composition).

As with the other methods described herein, the comparisons made in the methods of assessing efficacy (or relative efficacy) of compositions for treating kidney cancer may be carried out using various techniques, including simple comparisons, one or more statistical analyses, mathematical models, algorithms and combinations thereof. An example of a technique that may be used is determining the RCC score for a subject. Any suitable method may be used to analyze the biological samples in order to determine the level(s) of the one or more biomarkers in the samples. In addition, the level(s) of one or more biomarkers, including a combination of all of the biomarkers in Tables 1, 2, 4, 8, 10 and/or 11 or any fraction thereof, may be determined and used in methods of assessing efficacy (or relative efficacy) of compositions for treating kidney cancer.

Finally, the methods of assessing efficacy (or relative efficacy) of one or more compositions for treating kidney cancer may further comprise analyzing the biological sample to determine the level(s) of one or more non-biomarker compounds. The non-biomarker compounds may then be compared to reference levels of non-biomarker compounds for subjects having (or not having) kidney cancer.

G. Methods of Screening a Composition for Activity in Modulating Biomarkers Associated with Kidney Cancer

The identification of biomarkers for kidney cancer also allows for the screening of compositions for activity in modulating biomarkers associated with kidney cancer, which may be useful in treating kidney cancer. Methods of screening compositions useful for treatment of kidney cancer comprise assaying test compositions for activity in modulating the levels of one or more biomarkers in Tables 1, 2, 4, 8, 10 and/or 11. Such screening assays may be conducted in vitro and/or in vivo, and may be in any form known in the art useful for assaying modulation of such biomarkers in the presence of a test composition such as, for example, cell culture assays, organ culture assays, and in vivo assays (e.g., assays involving animal models).

In one embodiment, a method for screening a composition for activity in modulating one or more biomarkers of kidney cancer comprises (1) contacting one or more cells with a composition, (2) analyzing at least a portion of the one or more cells or a biological sample associated with the cells to determine the level(s) of one or more biomarkers of kidney cancer selected from Tables 1, 2, 4, 8, 10 and/or 11; and (3) comparing the level(s) of the one or more biomarkers with predetermined standard levels for the one or more biomarkers to determine whether the composition modulated the level(s) of the one or more biomarkers. As discussed above, the cells may be contacted with the composition in vitro and/or in vivo. The predetermined standard levels for the one or more biomarkers may be the levels of the one or more biomarkers in the one or more cells in the absence of the composition. The predetermined standard levels for the one or more biomarkers may also be the level(s) of the one or more biomarkers in control cells not contacted with the composition.

In addition, the methods may further comprise analyzing at least a portion of the one or more cells or a biological sample associated with the cells to determine the level(s) of one or more non-biomarker compounds of kidney cancer. The levels of the non-biomarker compounds may then be compared to predetermined standard levels of the one or more non-biomarker compounds.

Any suitable method may be used to analyze at least a portion of the one or more cells or a biological sample associated with the cells in order to determine the level(s) of the one or more biomarkers (or levels of non-biomarker compounds).

Suitable methods include chromatography (e.g., HPLC, gas chromatograph, liquid chromatography), mass spectrometry (e.g., MS, MS-MS), ELISA, antibody linkage, other immunochemical techniques, and combinations thereof. Further, the level(s) of the one or more biomarkers (or levels of non-biomarker compounds) may be measured indirectly, for example, by using an assay that measures the level of a compound (or compounds) that correlates with the level of the biomarker(s) (or non-biomarker compounds) that are desired to be measured.

H. Methods of Treating Kidney Cancer

The identification of biomarkers for kidney cancer also allows for the treatment of kidney cancer. For example, in order to treat a subject having kidney cancer, an effective amount of one or more kidney cancer biomarkers that are lowered in kidney cancer as compared to a healthy subject not having kidney cancer may be administered to the subject. The biomarkers that may be administered may comprise one or more of the biomarkers in Tables 1, 2, 4, 8, 10 and/or 11 that are decreased in kidney cancer. In some embodiments, the biomarkers that are administered are one or more biomarkers listed in Tables 1, 2, 4, 8, 10 and/or 11 that are decreased in kidney cancer and that have a p-value less than 0.10. In other embodiments, the biomarkers that are administered are one or biomarkers listed in Tables 1, 2, 4, 8, 10 and/or 11 that are decreased in kidney cancer by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, or by 100% (i.e., absent).

### III. OTHER METHODS

Other methods of using the biomarkers discussed herein are also contemplated. For example, the methods described in U.S. Pat. No. 7,005,255,

U.S. Pat. No. 7,329,489, U.S. Pat. No. 7,553,616, U.S. Pat. No. 7,550,260, U.S. Pat. No. 7,550,258, U.S. Pat. No. 7,635,556, U.S. patent application Ser. No. 11/728,826, U.S. patent application Ser. No. 12/463,690 and U.S. patent application Ser. No. 12/182,828 may be conducted using a small molecule profile comprising one or more of the biomarkers disclosed herein.

In any of the methods listed herein, the biomarkers that are used may be selected from those biomarkers in Tables 1, 2, 4, 8, 10 and/or 11 having p-values of less than 0.05. The biomarkers that are used in any of the methods described herein may also be selected from those biomarkers in Tables 1, 2, 4, 8, 10 and/or 11 that are decreased in kidney cancer (as compared to the control) or that are decreased in high stage (as compared to control or low stage) or that are decreased in more aggressive (as compared to control or less aggressive) by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, or by 100% (i.e., absent); and/or those biomarkers in Tables 1, 2, 4, 8, 10 and/or 11 that are increased in kidney cancer (as compared to the control or remission) or that are increased high stage (as compared to control or low stage) or that are increased in more aggressive (as compared to control or less aggressive) by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, by at least 100%, by at least 110%, by at least 120%, by at least 130%, by at least 140%, by at least 150%, or more.

### IV. EXAMPLES

The invention will be further explained by the following illustrative examples that are intended to be non-limiting.

I. General Methods

A. Identification of Metabolic profiles for kidney cancer

Each sample was analyzed to determine the concentration of several hundred metabolites. Analytical techniques such as GC-MS (gas chromatography-mass spectrometry) and LC-MS (liquid chromatography-mass spectrometry) were used to analyze the metabolites. Multiple aliquots were simultaneously, and in parallel, analyzed, and, after appropriate quality control (QC), the information derived from each analysis was recombined. Every sample was characterized according to several thousand characteristics, which ultimately amount to several hundred chemical species. The techniques used were able to identify novel and chemically unnamed compounds.

B. Statistical Analysis

The data was analyzed using T-tests to identify molecules present at differential levels in a definable population or subpopulation (e.g., biomarkers for kidney cancer biological samples compared to control biological samples or compared to patients in remission from kidney cancer) useful for distinguishing between the definable populations (e.g., kidney cancer and control). Other molecules in the definable population or subpopulation were also identified.

Data was also analyzed using Random Forest Analysis. Random Forests give an estimate of how well individuals in a new data set can be classified into existing groups. Random Forest Analysis creates a set of classification trees based on continual sampling of the experimental units and compounds. Then each observation is classified based on the majority votes from all the classification trees. In statistics, a classification tree classifies the observations into groups based on combinations of the variables (in this instance variables are metabolites or compounds). There are many variations on the algorithms used to create trees. A tree algorithm searches for the metabolite (compound) that provides the largest split between the two groups. This produces nodes. Then at each node, the metabolite that provides the best split is used and so on. If the node cannot be improved on, then it stops at that node and any observation in that node is classified as the majority group.

Random Forests classify based on a large number (e.g. thousands) of trees. A subset of compounds and a subset of observations are used to create each tree. The observations used to create the tree are called the in-bag samples, and the remaining samples are called the out-of-bag samples. The classification tree is created from the in-bag samples, and the out-of-bag samples are predicted from this tree. To get the final classification for an observation, the “votes” for each group are counted based on the times it was an out-of-bag sample. For example, suppose observation 1 was classified as a “Control” by 2,000 trees, but classified as “Disease” by 3,000 trees. Using “majority wins” as the criterion, this sample is classified as “Disease.”

The results of the Random Forest are summarized in a Confusion Matrix. The rows correspond to the true grouping, and the columns correspond to the classification from the random forest. Thus, the diagonal elements indicate the correct classifications. A 50% error would occur by random chance for 2 groups, 66.67% error for three groups by random chance, etc. The “Out-of-Bag” (OOB) Error rate gives an estimate of how accurately new observations can be predicted using the random forest model (e.g., whether a sample is from a diseased subject or a control subject).

It is also of interest to see which variables are more “important” in the final classifications. The “Importance Plot” shows the top compounds ranked in terms of their importance. There are different criteria for ranking the importance, but the general idea is that removing an important variable will cause a greater decrease in accuracy than a variable that is less important.

The data were also analyzed using a mixed model which consists of both fixed effect and random effect and is widely used for clustered data to build models that are useful to identify the biomarker compounds that are associated with kidney cancer. This method allows for the ability to control the known confounding factors (e.g., age, gender, BMI) to reduce the likelihood of a spurious relationship and thus reduce the probability of false positives. To assess biomarkers for tumor aggressiveness, Fisher's method was used following the mixed model analysis to combine the results of stage, grade and metastatic potential. Biomarker compounds that are useful to predict kidney cancer and that are positively or negatively correlated with kidney cancer were identified in these analyses.

C. Biomarker Identification

Various peaks identified in the analyses (e.g. GC-MS, LC-MS, LC-MS-MS), including those identified as statistically significant, were subjected to a mass spectrometry based chemical identification process.

### Example 1

### Intact Biopsy Tissue Biomarkers for Kidney Cancer

Biomarkers were discovered by (1) analyzing tissue samples from human subjects to determine the levels of metabolites in the samples and then (2) statistically analyzing the results to determine those metabolites that were differentially present in the kidney cancer tissue samples compared to the benign tissue samples.

Six kidney cancer positive and 6 patient-matched non-cancer human kidney core biopsies were obtained post-nephrectomy using an 18 gauge biopsy gun and placed into cryovials (Nalgene) containing 2 ml of 80% methanol. A single biopsy was placed in each vial and incubated for 24-72 hours at room temperature (22-24° C.). Following incubation, the tissues were removed from the solvent for histological analysis, and the solvent was prepared for metabolomics analysis. The cancer status of the sample was verified by histopathology analysis. Histological analysis was performed by a board-certified pathologist.

For metabolomics analysis, the solvent extracts were evaporated to dryness under a stream of nitrogen gas at 40° C. in a Turbovap LV evaporator (Zymark). The dried extracts were reconstituted in 550 μl methanol:water (80:20) containing recovery standards (D,L-2-fluorophenylglycine, D,L-4-chlorophenylalanine, tridecanoic acid, D6 cholesterol). The reconstituted solution was analyzed by metabolomics.

After the levels of metabolites were determined, statistical analysis was performed to identify metabolites that were significantly altered in the kidney cancer samples compared to the patient-matched non-cancer samples. The results of the matched pairs t-test analysis showed that 91 metabolites were significantly (p<0.1) altered in kidney cancer samples compared to the non-cancer samples. Table 1 lists the identified biomarkers having a p-value of less than 0.1. Table 1 includes, for each listed biomarker, the biochemical name of the biomarker, an indication of the percentage difference in the cancer sample mean as compared to the non-cancer sample mean (positive values represent an increase in kidney cancer, and negative values represent a decrease in kidney cancer), the p-value, and the q-value determined in the statistical analysis of the data concerning the biomarkers. Also included in Table 1 are: the identifier for that biomarker compound in the Kyoto Encyclopedia of Genes and Genomes (KEGG), if available; and the identifier for that biomarker compound in the Human Metabolome Database (HMDB), if available.

TABLE 1
Kidney Cancer Tissue Biomarkers, p < 0.1

| | % change | | | | |
|---|---|---|---|---|---|
| Biochemical Name | in cancer | P-Value | Q-Value | Kegg | HMDB |
| glycerate | 175% | 0.0242 | 0.065 | C00258 | HMDB00139 |
| sphingosine | 716% | 0.0212 | 0.065 | C00319 | HMDB00252 |
| phosphoethanolamine | 779% | 0.0365 | 0.0667 | C00346 | HMDB00224 |
| choline phosphate | 229% | 0.0576 | 0.0798 | | |
| pyrophosphate (PPi) | 446% | 0.0611 | 0.082 | C00013 | HMDB00250 |
| 2-oleoylglycerophosphoethanolamine | 374% | 0.0011 | 0.0522 | | |
| 2-docosahexaenoylglycerophosphocholine | 124% | 0.0059 | 0.065 | | |
| 2-docosahexaenoylglycerophosphoethanolamine | 379% | 0.0153 | 0.065 | | |
| glutathione, oxidized (GSSG) | 433% | 0.0158 | 0.065 | C00127 | HMDB03337 |
| 2-arachidonoylglycerophosphoethanolamine | 731% | 0.0172 | 0.065 | | |
| 2-arachidonoylglycerophosphocholine | 701% | 0.0236 | 0.065 | | |
| 2-oleoylglycerophosphocholine | 327% | 0.0251 | 0.065 | | |
| 1-arachidonoylglycerophosphoinositol | 160% | 0.0359 | 0.0667 | | |
| nicotinamide adenine dinucleotide | 188% | 0.0366 | 0.0667 | C00003 | HMDB00902 |
| (NAD+) | | | | | |
| 2-linoleoylglycerophosphocholine | 185% | 0.0616 | 0.082 | | |
| 1-arachidonoylglycerophosphoethanolamine | 192% | 0.0724 | 0.093 | | HMDB11517 |
| methyl-alpha-glucopyranoside | 354% | <0.001 | 0.0272 | C04942, | |
| | | | | C02603 | |
| margarate (17:0) | 54% | 0.0061 | 0.065 | | HMDB02259 |
| cholesterol | 75% | 0.0071 | 0.065 | C00187 | HMDB00067 |
| stearate (18:0) | 38% | 0.0073 | 0.065 | C01530 | HMDB00827 |
| palmitate (16:0) | 25% | 0.0086 | 0.065 | C00249 | HMDB00220 |
| deoxycarnitine | 186% | 0.0114 | 0.065 | C01181 | HMDB01161 |
| arginine | 26% | 0.0208 | 0.065 | C00062 | HMDB00517 |
| 2-palmitoylglycerophosphocholine | 342% | 0.0223 | 0.065 | | |
| 1-palmitoylglycerophosphocholine | 522% | 0.0224 | 0.065 | | |
| betaine | 139% | 0.0242 | 0.065 | | HMDB00043 |
| 1-linoleoylglycerophosphocholine | 450% | 0.0282 | 0.066 | C04100 | |
| 1-oleoylglycerophosphocholine | 320% | 0.0304 | 0.0667 | | |
| uridine | 60% | 0.0316 | 0.0667 | C00299 | HMDB00296 |
| ornithine | 73% | 0.0342 | 0.0667 | C00077 | HMDB03374 |
| butyrylcarnitine | 163% | 0.0344 | 0.0667 | | |
| phosphate | 102% | 0.0348 | 0.0667 | C00009 | HMDB01429 |
| 1-linoleoylglycerophosphoethanolamine | 128% | 0.0363 | 0.0667 | | HMDB11507 |
| urea | 417% | 0.0413 | 0.069 | C00086 | HMDB00294 |
| oleoylcarnitine | 1134% | 0.0454 | 0.0724 | | HMDB05065 |
| 1-arachidonoylglycerophosphocholine | 110% | 0.0496 | 0.0746 | C05208 | |
| phosphoglycerate (2 or 3) | 43% | 0.0497 | 0.0746 | | |
| palmitoylcarnitine | 1333% | 0.0501 | 0.0746 | | |
| methylphosphate | 141% | 0.0575 | 0.0798 | | |
| eicosenoate (20:1n9 or 11) | 95% | 0.0623 | 0.082 | | HMDB02231 |
| inositol 1-phosphate (I1P) | 430% | 0.0693 | 0.0901 | | HMDB00213 |
| ophthalmate | 284% | 0.0867 | 0.1061 | | HMDB05765 |
| 1-stearoylglycerophosphocholine | 319% | 0.0902 | 0.1081 | | |
| 1-palmitoylplasmenylethanolamine | 114% | 0.0919 | 0.1081 | | |
| trans-4-hydroxyproline | 227% | 0.0924 | 0.1081 | C01157 | HMDB00725 |
| 6-phosphogluconate | 235% | 0.0971 | 0.1124 | C00345 | HMDB01316 |
| 2-hydroxybutyrate (AHB) | 41% | 0.002 | 0.0522 | C05984 | HMDB00008 |
| glycerol | 60% | 0.0037 | 0.0648 | C00116 | HMDB00131 |
| 2-hydroxyglutarate | 205% | 0.0295 | 0.066 | C02630 | HMDB00606 |
| stearoylcarnitine | 548% | 0.0337 | 0.0667 | | HMDB00848 |
| N-acetylneuraminate | 365% | 0.0424 | 0.0698 | C00270 | HMDB00230 |
| 1,5-anhydroglucitol (1,5-AG) | 16% | 0.076 | 0.0963 | C07326 | HMDB02712 |
| 5-oxoproline | 93% | 0.002 | 0.0522 | C01879 | HMDB00267 |
| 3-hydroxybutyrate (BHBA) | 85% | 0.0029 | 0.0602 | C01089 | HMDB00357 |
| lactate | 89% | 0.0075 | 0.065 | C00186 | HMDB00190 |
| tyrosine | 55% | 0.0076 | 0.065 | C00082 | HMDB00158 |
| isoleucine | 56% | 0.0098 | 0.065 | C00407 | HMDB00172 |
| leucine | 48% | 0.0102 | 0.065 | C00123 | HMDB00687 |
| valine | 36% | 0.0103 | 0.065 | C00183 | HMDB00883 |
| 3-dehydrocarnitine | 172% | 0.0132 | 0.065 | C02636 | HMDB12154 |
| lysine | 38% | 0.0139 | 0.065 | C00047 | HMDB00182 |
| 3-aminoisobutyrate | 418% | 0.0144 | 0.065 | C05145 | HMDB03911 |
| acetylcarnitine | 233% | 0.0149 | 0.065 | C02571 | HMDB00201 |
| adenine | 96% | 0.0171 | 0.065 | C00147 | HMDB00034 |
| serine | 131% | 0.0178 | 0.065 | C00065 | HMDB03406 |
| phenylalanine | 50% | 0.0226 | 0.065 | C00079 | HMDB00159 |
| 5-methylthioadenosine (MTA) | 270% | 0.0229 | 0.065 | C00170 | HMDB01173 |
| tryptophan | 56% | 0.0239 | 0.065 | C00078 | HMDB00929 |
| succinate | 206% | 0.0248 | 0.065 | C00042 | HMDB00254 |
| hexanoylcarnitine | 187% | 0.0253 | 0.065 | C01585 | HMDB00705 |
| carnitine | 79% | 0.0253 | 0.065 | | |
| pyruvate | 431% | 0.0254 | 0.065 | C00022 | HMDB00243 |
| proline | 107% | 0.0259 | 0.065 | C00148 | HMDB00162 |
| stachydrine | 82% | 0.0272 | 0.066 | C10172 | HMDB04827 |
| histidine | 41% | 0.028 | 0.066 | C00135 | HMDB00177 |
| pyroglutamine | 255% | 0.0295 | 0.066 | | |
| 5,6-dihydrouracil | 84% | 0.037 | 0.0667 | C00429 | HMDB00076 |
| 2-aminobutyrate | 66% | 0.0379 | 0.0667 | CO2261 | HMDB00650 |
| alanine | 168% | 0.0383 | 0.0667 | C00041 | HMDB00161 |
| malate | 321% | 0.0389 | 0.0667 | C00149 | HMDB00156 |
| glutamine | 40% | 0.0393 | 0.0667 | C00064 | HMDB00641 |
| glycine | 114% | 0.0446 | 0.0723 | C00037 | HMDB00123 |
| threonine | 58% | 0.0462 | 0.0726 | C00188 | HMDB00167 |
| creatine | 127% | 0.0503 | 0.0746 | C00300 | HMDB00064 |
| hypoxanthine | 53% | 0.0516 | 0.0754 | C00262 | HMDB00157 |
| erythritol | 133% | 0.0548 | 0.079 | C00503 | HMDB02994 |
| glycerol 3-phosphate (G3P) | 89% | 0.0573 | 0.0798 | C00093 | HMDB00126 |
| glutamate | 158% | 0.0613 | 0.082 | C00025 | HMDB03339 |
| octanoylcarnitine | 55% | 0.0771 | 0.0966 | | |
| choline | 61% | 0.0842 | 0.1042 | | |
| glycolate (hydroxyacetate) | 33% | 0.0924 | 0.1081 | C00160 | HMDB00115 |

Listed in Table 2 are biomarkers that were identified as differentially present between kidney cancer samples compared to the patient-matched non-cancer samples where p>0.1. All of the biomarkers in Table 2 differentially increase or decrease at least 5% in the kidney cancer samples. Table 2 includes, for each listed biomarker, the biochemical name of the biomarker, an indication of the percentage difference in the cancer sample mean as compared to the benign sample mean (positive values represent an increase in cancer, and negative values represent a decrease in cancer), the p-value and the q-value. Also included in Table 2 are: the identifier for that biomarker compound in the Kyoto Encyclopedia of Genes and Genomes (KEGG), if available; and the identifier for that biomarker compound in the Human Metabolome Database (HMDB), if available.

TABLE 2
Kidney Cancer Biomarkers, p > 0.1

| | % change | | | | |
|---|---|---|---|---|---|
| Biochemical Name | in cancer | P-Value | Q-Value | Kegg | HMDB |
| 1,2-propanediol | 182% | 0.3703 | 0.2515 | C00717, | HMDB01881 |
| | | | | C02912, | |
| | | | | C00583, | |
| | | | | C01506, | |
| | | | | C02917 | |
| glutamate, gamma-methyl ester | 483% | 0.1085 | 0.1241 | | |
| Isobar: fructose 1,6-diphosphate, glucose | 220% | 0.1099 | 0.1241 | | |
| 1,6-diphosphate | | | | | |
| cytidine 5′-monophosphate (5′-CMP) | 48% | 0.1125 | 0.1241 | C00055 | HMDB00095 |
| adrenate (22:4n6) | 107% | 0.1219 | 0.1301 | C16527 | HMDB02226 |
| taurine | 82% | 0.1301 | 0.1342 | C00245 | HMDB00251 |
| 1-stearoylglycerophosphoinositol | 133% | 0.1385 | 0.1376 | | |
| inosine | 71% | 0.1424 | 0.1401 | | |
| hypotaurine | 28% | 0.1473 | 0.1436 | C00519 | HMDB00965 |
| ethanolamine | 398% | 0.1496 | 0.1444 | C00189 | HMDB00149 |
| adenosine 5'-monophosphate (AMP) | 307% | 0.1527 | 0.1448 | C00020 | HMDB00045 |
| 10-heptadecenoate (17:1n7) | 43% | 0.1647 | 0.1546 | | |
| 2-linoleoylglycerophosphoethanolamine | 322% | 0.1659 | 0.1546 | | |
| 2-docosapentaenoylglycerophosphoethanolamine | 529% | 0.1686 | 0.1557 | | |
| glycylleucine | 46% | 0.181 | 0.1657 | C02155 | HMDB00759 |
| nicotinamide | 157% | 0.192 | 0.1728 | C00153 | HMDB01406 |
| 1-oleoylglycerophosphoethanolamine | 113% | 0.1993 | 0.1763 | | HMDB11506 |
| glucose 1-phosphate | 126% | 0.2102 | 0.1813 | C00103 | HMDB01586 |
| palmitoyl sphingomyelin | 78% | 0.2132 | 0.1814 | | |
| 1-oleoylglycerol (1-monoolein) | −24% | 0.2137 | 0.1814 | | HMDB11567 |
| glutathione, reduced (GSH) | 1351% | 0.2199 | 0.1837 | C00051 | HMDB00125 |
| ergothioneine | 111% | 0.2236 | 0.1839 | C05570 | HMDB03045 |
| nicotinamide adenine dinucleotide | 67% | 0.2373 | 0.1883 | C00004 | HMDB01487 |
| reduced (NADH) | | | | | |
| 1-stearoylglycerophosphoethanolamine | 163% | 0.2383 | 0.1883 | | HMDB11130 |
| pentadecanoate (15:0) | 28% | 0.2412 | 0.1883 | C16537 | HMDB00826 |
| methyl palmitate (15 or 2) | 20% | 0.2414 | 0.1883 | | |
| 4-hydroxybutyrate (GHB) | 254% | 0.2839 | 0.2165 | C00989 | HMDB00710 |
| dihomo-linoleate (20:2n6) | 79% | 0.2917 | 0.2194 | C16525 | |
| cysteine-glutathione disulfide | −19% | 0.307 | 0.2292 | | HMDB00656 |
| glucose-6-phosphate (G6P) | 383% | 0.3097 | 0.2296 | C00668 | HMDB01401 |
| heme | 1219% | 0.3325 | 0.2448 | | |
| citalopram | 49% | 0.3632 | 0.2483 | C07572 | HMDB05038 |
| S-adenosylmethionine (SAM) | 11% | 0.3632 | 0.2483 | | |
| gamma-glutamylglutamate | 85% | 0.3932 | 0.2637 | | |
| glycerol 2-phosphate | 113% | 0.4122 | 0.2713 | C02979, | HMDB02520 |
| | | | | D01488 | |
| docosapentaenoate (n3 DPA; 22:5n3) | 23% | 0.4656 | 0.2989 | C16513 | HMDB01976 |
| 1-behenoyl glycerol (1-monobehenin) | −6% | 0.4747 | 0.3029 | | |
| oleate (18:1n9) | 18% | 0.4965 | 0.3111 | C00712 | HMDB00207 |
| citrulline | 14% | 0.5164 | 0.3198 | C00327 | HMDB00904 |
| arabitol | −6% | 0.5263 | 0.324 | C00474 | HMDB01851 |
| caproate (6:0) | 350% | 0.5763 | 0.3507 | C01585 | HMDB00535 |
| arachidonate (20:4n6) | 6% | 0.5829 | 0.3527 | C00219 | HMDB01043 |
| octaethylene glycol | 58% | 0.6077 | 0.3615 | | |
| docosapentaenoate (n6 DPA; 22:5n6) | 17% | 0.6078 | 0.3615 | C06429 | HMDB13123 |
| 1 -palmitoylglycerophosphoethanolamine | 57% | 0.6128 | 0.3623 | | HMDB11503 |
| 2-hydroxypalmitate | 29% | 0.639 | 0.3737 | | |
| linoleate (18:2n6) | 12% | 0.6593 | 0.3813 | C01595 | HMDB00673 |
| heptaethylene glycol | 66% | 0.6691 | 0.3849 | | |
| 13-methylmyristic acid | 62% | 0.6781 | 0.3864 | | |
| 1-myristoylglycerol (1-monomyristin) | 41% | 0.679 | 0.3864 | | HMDB11561 |
| 2-hydroxystearate | 34% | 0.7269 | 0.4071 | C03045 | |
| pelargonate (9:0) | 18% | 0.7533 | 0.413 | C01601 | HMDB00847 |
| tetraethylene glycol | 767% | 0.7963 | 0.4323 | | |
| myristate (14:0) | 7% | 0.7967 | 0.4323 | C06424 | HMDB00806 |
| 2-ethylhexanoate | 56% | 0.803 | 0.4326 | | |
| heptanoate (7:0) | 15% | 0.8149 | 0.4352 | C17714 | HMDB00666 |
| palmitoleate (16:1n7) | 32% | 0.8214 | 0.4352 | C08362 | HMDB03229 |
| hexaethylene glycol | 111% | 0.8227 | 0.4352 | | |
| 2-stearoylglycerol (2-monostearin) | 8% | 0.8349 | 0.4391 | | |
| triethyleneglycol | 323% | 0.8384 | 0.4391 | | |
| 1-heptadecanoylglycerol (1-monoheptadecanoin) | 35% | 0.8509 | 0.4403 | | |
| docosahexaenoate (DHA; 22:6n3) | 19% | 0.8694 | 0.4443 | C06429 | HMDB02183 |
| caprate (10:0) | 10% | 0.9059 | 0.4607 | C01571 | HMDB00511 |
| 1-stearoyl glycerol (1-monostearin) | 15% | 0.9147 | 0.4629 | D01947 | |
| dihomo-linolenate (20:3n3 or n6) | 34% | 0.9299 | 0.4684 | C03242 | HMDB02925 |
| linoleamide (18:2n6) | 84% | 0.9344 | 0.4684 | | |
| caprylate (8:0) | 26% | 0.9446 | 0.4694 | C06423 | HMDB00482 |
| linolenate [alpha or gamma; (18:3n3 or 6)] | 15% | 0.9454 | 0.4694 | C06427 | HMDB01388 |
| 1-octadecanol | 7% | 0.9575 | 0.4732 | D01924 | HMDB02350 |
| pentaethylene glycol | 199% | 0.9722 | 0.4783 | | |
| n-Butyl Oleate | 20% | 0.9868 | 0.4832 | | |
| 1-palmitoylglycerol (1-monopalmitin) | 14% | 0.997 | 0.4837 | | |
| C-glycosyltryptophan | 38% | 0.125 | 0.1303 | | |
| trizma acetate | −28% | 0.2347 | 0.1883 | C07182 | |
| 4-methyl-2-oxopentanoate | 37% | 0.4105 | 0.2713 | C00233 | HMDB00695 |
| glucose | 297% | 0.112 | 0.1241 | C00293 | HMDB00122 |
| methionine | 10% | 0.1131 | 0.1241 | C00073 | HMDB00696 |
| glycerophosphorylcholine (GPC) | 41% | 0.1199 | 0.1301 | C00670 | HMDB00086 |
| aspartate | 197% | 0.1223 | 0.1301 | C00049 | HMDB00191 |
| ribitol | 195% | 0.1247 | 0.1303 | C00474 | HMDB00508 |
| beta-alanine | 93% | 0.1326 | 0.1355 | C00099 | HMDB00056 |
| fumarate | 245% | 0.1356 | 10.1363 | C00122 | HMDB00134 |
| citrate | 55% | 0.136 | 0.1363 | C00158 | HMDB00094 |
| propionylcarnitine | 167% | 0.1509 | 0.1444 | C03017 | HMDB00824 |
| uracil | 54% | 0.185 | 0.1679 | C00106 | HMDB00300 |
| scyllo-inositol | 234% | 0.1982 | 0.1763 | C06153 | HMDB06088 |
| pantothenate | 81% | 0.2079 | 0.1813 | C00864 | HMDB00210 |
| sorbitol | 75% | 0.2087 | 0.1813 | C00794 | HMDB00247 |
| isobutyrylcarnitine | 83% | 0.2183 | 0.1837 | | |
| kynurenine | 60% | 0.2223 | 0.1839 | C00328 | HMDB00684 |
| threonate | 103% | 0.2279 | 0.185 | C01620 | HMDB00943 |
| gluconate | 33% | 0.2285 | 0.185 | C00257 | HMDB00625 |
| 2-aminoadipate | 138% | 0.2719 | 0.2105 | C00956 | HMDB00510 |
| xanthine | 72% | 0.2766 | 0.2126 | C00385 | HMDB00292 |
| erythronate | 83% | 0.2905 | 0.2194 | | HMDB00613 |
| pipecolate | 41% | 0.3578 | 0.2483 | C00408 | HMDB00070 |
| 3-methyl-2-oxovalerate | 30% | 0.3632 | 0.2483 | C00671 | HMDB03736 |
| p-acetamidophenylglucuronide | 6% | 0.3632 | 0.2483 | | HMDB10316 |
| glutaroyl carnitine | −7% | 0.3632 | 0.2483 | | HMDB13130 |
| pseudouridine | −13% | 0.3632 | 0.2483 | C02067 | HMDB00767 |
| myo-inositol | 186% | 0.3752 | 0.2532 | C00137 | HMDB00211 |
| pro-hydroxy-pro | −12% | 0.4123 | 0.2713 | | HMDB06695 |
| fructose | 186% | 0.4202 | 0.2747 | C00095 | HMDB00660 |
| adenosine | 97% | 0.431 | 0.2801 | C00212 | HMDB00050 |
| p-cresol sulfate | −5% | 0.4362 | 0.2817 | C01468 | |
| gamma-aminobutyrate (GABA) | −5% | 0.4786 | 0.3035 | C00334 | HMDB00112 |
| 1-methylnicotinamide | 19% | 0.4853 | 0.3059 | C02918 | HMDB00699 |
| benzoate | 43% | 0.5148 | 0.3198 | C00180 | HMDB01870 |
| mannitol | 6% | 0.616 | 0.3623 | C00392 | HMDB00765 |
| xylitol | 7% | 0.687 | 0.3888 | C00379 | HMDB00568 |
| N-acetylaspartate (NAA) | 12% | 0.7133 | 0.4015 | C01042 | HMDB00812 |
| phenylacetylglutamine | 186% | 0.7351 | 0.4091 | C05597 | HMDB06344 |
| urate | 60% | 0.7423 | 0.4091 | C00366 | HMDB00289 |
| creatinine | 9% | 0.8054 | 0.4326 | C00791 | HMDB00562 |
| cysteine | 57% | 0.8551 | 0.4403 | C00097 | HMDB00574 |
| metoprolol acid metabolite | 40% | 0.9946 | 0.4837 | | |

### Example 2

### Statistical Analysis for the Classification of Subjects Based on Tissue Biomarkers

The data obtained in Example 1 concerning biopsy samples was used to create a statistical (mathematical) model to classify the samples into kidney cancer or non-cancer groups.

Random Forest Analysis was used to classify kidney samples into kidney cancer positive (kidney cancer) or cancer negative groups. Random Forests give an estimate of how well individuals in a new data set can be classified into each group. This is in contrast to a t-test, which tests whether or not the unknown means for two populations are different. Random forests create a set of classification trees based on continual sampling of the experimental units and compounds. Then each observation is classified based on the majority votes from all the classification trees.

Random forest results show that the samples can be classified correctly with 83% prediction accuracy. The Confusion Matrix presented in Table 3 shows the number of samples predicted for each classification and the actual in each group (Kidney Cancer or Non-Cancer). The “Out-of-Bag” (OOB) Error rate gives an estimate of how accurately new observations can be predicted using the Random Forest Model (e.g., whether a sample contains tumor (cancer-positive) or is cancer-negative). The OOB error from this Random Forest was approximately 17%, and the model estimated that, when used on a new set of samples, the identity of kidney cancer positive samples could be predicted correctly 67% of the time and non-cancer samples could be predicted correctly 100% of the time.

TABLE 3
Random Forest Classification of cancer-positive and benign kidney tissue
samples.

| | | Random Forest Prediction | | Class |
|---|---|---|---|---|
| | | Kidney Cancer | Non-Cancer | Error |
| Histologically | Kidney Cancer | 4 | 2 | 0.333 |
| confirmed | Acutal | | | |
| patient | Non-Cancer | 0 | 6 | 0 |
| samples | Acutal | | | |

Predictive accuracy = 83%

Based on the OOB Error rate of 17%, the Random Forest model that was created predicted whether a sample was kidney cancer positive with about 83% accuracy based on the levels of the biomarkers measured in samples from the subjects. Exemplary biomarkers for distinguishing the groups are oxidized glutathione (GSSG), proline, 2-oleoylglycerophosphoethanolamine, 2-aminobutyrate, sphingosine, 3-dehydrocamitine, 2-docosahexaenoylglycerophosphocholine, 2-linoleoylglycerophosphocholine, phosphoethanolamine, glutamate, pyrophosphate (PPi), nicotinamide-adenine-dinucleotide (NAD+), 3-aminoisobutyrate, 2-arachidonoylglycerophosphoethanolamine, 2-arachidonoylglycerophosphocholine, 2-oleoylglycerophosphocholine, glycerate, choline-phosphate, pyruvate, 1-arachidonoylglycerophosphoethanolamine, adenine, 1-2-propanediol, 2-docosahexaenoylglycerophosphoethanolamine, 2-hydroxybutyrate (AHB), creatine, glycolate (hydroxyacetate), malate, 5-methylthioadenosine (MTA), stearolycarnitine, and 1-arachidonoylglycerophosphoinositol.

The Random Forest analysis demonstrated that by using the biomarkers, kidney cancer positive samples were distinguished from non-cancer samples with 67% sensitivity, 100% specificity, 100% Positive Predictive Value (PPV), and 75% Negative Predictive Value (NPV).

In addition, Principal Component Analysis (PCA) was carried out using the biomarkers where p<0.05 obtained from biopsy samples in Example 1 to classify the samples as non-cancer or Kidney Cancer (RCC).

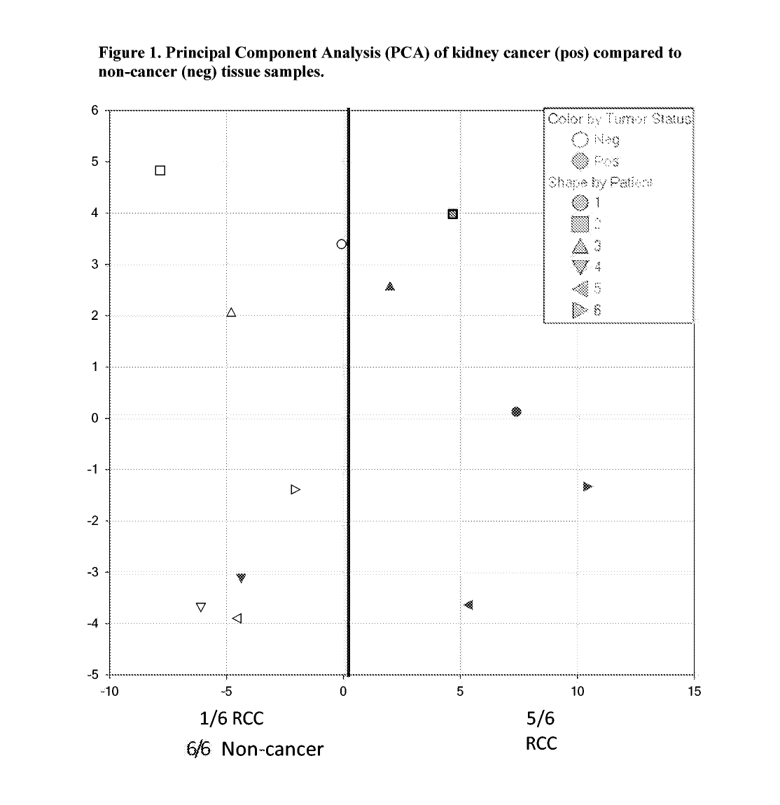

Using the mathematical model created using PCA, it was found that 6 of 6 cancer-negative samples were correctly classified as cancer negative while 5 of 6 kidney cancer-positive samples were correctly classified as kidney cancer based on the biomarker abundance. A graphical depiction of the PCA results is presented in FIG. 1.

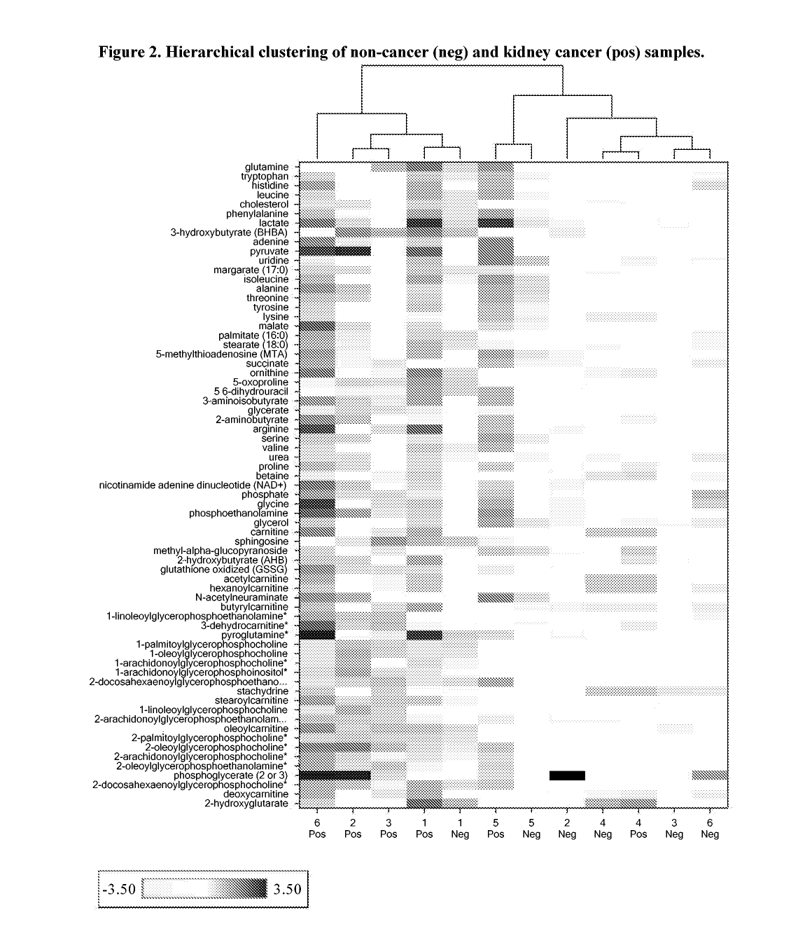

Hierarchical clustering (Euclidean distance) using the biomarkers where p<0.05 identified from biopsy samples in Example 1 was also used to classify the subjects. This analysis resulted in the subjects being divided into two distinct groups. One group consisted of four cancer biopsies and one non-cancer biopsy, and the other group consisted of two cancer biopsies and five non-cancer biopsies. These data suggest that there are multiple metabolic types of kidney disease and/or kidney cancer that can be distinguished using tissue biopsy biomarker metabolite levels. For example, the cancer-containing samples identified in the second group may have a less aggressive faun of kidney cancer or may be at an earlier stage of cancer. Distinguishing between types of cancer (e.g., less vs. more aggressive) and stage of cancer may be valuable information to a doctor determining a course of treatment. FIG. 2 provides a graphical depiction of the results of the hierarchical clustering.

### Example 3

### Tissue Biomarkers for Kidney Cancer

Biomarkers were discovered by (1) analyzing different groups of tissue samples from human subjects to determine the levels of metabolites in the samples and then (2) statistically analyzing the results to determine those metabolites that are differentially present in the following groups: normal tissue compared to tumor tissue; early stage (T1) cancer tissue compared to normal tissue; and later stage (T3) cancer tissue compared to normal tissue.

The samples used for the analysis were matched pairs of RCC tumor and adjacent normal kidney tissue collected from 140 subjects with RCC. Subjects were further divided based on tumor stage with 43 subjects having Stage 1 (T1), 13 subjects with Stage 2 (T2), 80 subjects with Stage 3 (T3) and 4 subjects with Stage 4 (T4) kidney cancer.

After the levels of metabolites were determined, the data were analyzed using Welch's two-sample t-tests. Three comparisons were used to identify biomarkers for kidney cancer: Kidney cancer vs. Normal; T1 Kidney cancer vs. Normal; T3 Kidney cancer vs. Normal. As listed in Table 4 below, the analysis of named compounds resulted in the identification of biomarkers that are differentially present between a) kidney cancer and Normal tissue b) early stage (T1) kidney cancer and Normal tissue and/or c) later stage (T3) kidney cancer and Normal tissue.

Table 4 includes, for each biomarker, the biochemical name of the biomarker, the fold change (FC) of the biomarker in kidney cancer compared to non-kidney cancer samples (Tumor/Normal, T1 Tumor/T1 Normal and T3 Tumor/T3 Normal) which is the ratio of the mean level of the biomarker in kidney cancer samples as compared to the non-kidney cancer mean level and the p-value determined in the statistical analysis of the data concerning the biomarkers. Bold values indicate a fold of change with a p-value of ≦0.1.

TABLE 4
Tissue Biomarkers for Kidney Cancer

| | Tumor | | T1 Tumor | | T3 Tumor | |
|---|---|---|---|---|---|---|
| | N̅o̅r̅m̅a̅l̅ | | T̅1̅ N̅o̅r̅m̅a̅l̅ | | T̅3̅ N̅o̅r̅m̅a̅l̅ | |
| Biochemical Name | FC | p-value | FC | p-value | FC | p-value |
| eicosenoate (20:1n9 or 11) | 4.91 | p < 0.0001 | 5.42 | p < 0.0001 | 4.66 | p < 0.0001 |
| arachidonate (20:4n6) | 0.3 | p < 0.0001 | 0.29 | p < 0.0001 | 0.31 | p < 0.0001 |
| mannose-6-phosphate | 8.39 | p < 0.0001 | 5.38 | 3.81E−09 | 9.28 | p < 0.0001 |
| alpha-tocopherol | 8.76 | p < 0.0001 | 8.84 | 2.74E−12 | 9.21 | p < 0.0001 |
| flavin adenine dinucleotide (FAD) | 0.24 | p < 0.0001 | 0.23 | 7.43E−12 | 0.25 | p < 0.0001 |
| fructose-6-phosphate | 6.92 | p < 0.0001 | 6.1 | 2.00E−15 | 7.02 | p < 0.0001 |
| maltose | 17.03 | p < 0.0001 | 13.98 | p < 0.0001 | 17.5 | p < 0.0001 |
| maltotriose | 21.95 | p < 0.0001 | 14.41 | p < 0.0001 | 26.14 | p < 0.0001 |
| fructose 1-phosphate | 9.62 | p < 0.0001 | 10.09 | 9.38E−11 | 9.48 | p < 0.0001 |
| maltotetraose | 13.04 | p < 0.0001 | 8.7 | 2.52E−11 | 14.42 | p < 0.0001 |
| 1-stearoylglycerophosphoinositol | 0.29 | p < 0.0001 | 0.22 | 1.00E−15 | 0.33 | p < 0.0001 |
| methyl-alpha-glucopyranoside | 4.65 | p < 0.0001 | 3.85 | 1.51E−07 | 5.32 | p < 0.0001 |
| glucose-6-phosphate (G6P) | 9.38 | p < 0.0001 | 6.63 | 3.40E−14 | 10.24 | p < 0.0001 |
| 1-stearoylglycerophosphoethanolamine | 0.1 | p < 0.0001 | 0.07 | p < 0.0001 | 0.11 | p < 0.0001 |
| 1-palmitoylglycerophosphoinositol | 0.21 | p < 0.0001 | 0.19 | 3.00E−15 | 0.23 | p < 0.0001 |
| 1-oleoylglycerophosphoethanolamine | 0.05 | p < 0.0001 | 0.04 | p < 0.0001 | 0.06 | p < 0.0001 |
| 1-palmitoylglycerophosphoethanolamine | 0.03 | p < 0.0001 | 0.02 | p < 0.0001 | 0.03 | p < 0.0001 |
| 2-oleoylglycerophosphoethanolamine | 0.09 | p < 0.0001 | 0.08 | p < 0.0001 | 0.1 | p < 0.0001 |
| 2-palmitoylglycerophosphoethanolamine | 0.03 | p < 0.0001 | 0.02 | p < 0.0001 | 0.03 | p < 0.0001 |
| 1-oleoylglycerophosphoinositol | 0.34 | p < 0.0001 | 0.33 | 1.42E−12 | 0.35 | p < 0.0001 |
| gamma-glutamylglutamate | 4.6 | p < 0.0001 | 7.25 | 2.68E−12 | 3.7 | 1.42E−13 |
| ergothioneine | 4.22 | p < 0.0001 | 3.8 | 6.58E−12 | 4.61 | p < 0.0001 |
| arabitol | 0.38 | p < 0.0001 | 0.45 | 5.06E−08 | 0.37 | p < 0.0001 |
| 1-palmitoylplasmenylethanolamine | 0.12 | p < 0.0001 | 0.1 | 1.00E−15 | 0.14 | p < 0.0001 |
| phosphoenolpyruvate (PEP) | 0.37 | p < 0.0001 | 0.36 | 3.30E−06 | 0.37 | 1.66E−09 |
| putrescine | 4.65 | p < 0.0001 | 5.7 | 4.04E−06 | 4.94 | 1.00E−15 |
| inositol 1-phosphate (I1P) | 0.4 | p < 0.0001 | 0.45 | 7.10E−10 | 0.36 | p < 0.0001 |
| ethanolamine | 0.4 | p < 0.0001 | 0.39 | 5.62E−07 | 0.42 | 1.13E−08 |
| erucate (22:1n9) | 4.63 | p < 0.0001 | 5.69 | 3.03E−12 | 4.17 | 8.60E−14 |
| 3,4-dihydroxyphenethyleneglycol | 0.27 | p < 0.0001 | 0.25 | 6.73E−12 | 0.28 | 1.60E−14 |
| N-acetylalanine | 0.44 | p < 0.0001 | 0.42 | 1.19E−13 | 0.45 | p < 0.0001 |
| N-acetylmethionine | 2.46 | p < 0.0001 | 2.02 | 7.54E−05 | 2.7 | 1.00E−15 |
| pyridoxal | 0.36 | p < 0.0001 | 0.32 | 1.21E−13 | 0.41 | p < 0.0001 |
| urea | 0.52 | p < 0.0001 | 0.6 | 0.0001 | 0.53 | 6.12E−10 |
| glutathione, reduced (GSH) | 37.54 | p < 0.0001 | 9.03 | 1.04E−05 | 43.43 | 2.40E−14 |
| asparagine | 0.38 | p < 0.0001 | 0.34 | 5.91E−10 | 0.41 | 3.03E−09 |
| glucose 1-phosphate | 9.38 | p < 0.0001 | 9.92 | 0.00E+00 | 8.26 | p < 0.0001 |
| dihomo-linoleate (20:2n6) | 2.57 | p < 0.0001 | 2.57 | 2.69E−09 | 2.66 | p < 0.0001 |
| 5-methyltetrahydrofolate (5MeTHF) | 0.22 | p < 0.0001 | 0.2 | 1.00E−15 | 0.24 | p < 0.0001 |
| glycylvaline | 0.4 | p < 0.0001 | 0.38 | 6.70E−14 | 0.44 | 6.28E−12 |
| eicosapentaenoate (EPA; 20:5n3) | 0.45 | p < 0.0001 | 0.43 | 6.54E−09 | 0.48 | 3.89E−08 |
| 1-oleoylglycerophosphoserine | 0.45 | p < 0.0001 | 0.38 | 5.57E−10 | 0.52 | 1.45E−12 |
| docosahexaenoate (DHA; 22:6n3) | 0.4 | p < 0.0001 | 0.37 | 3.50E−14 | 0.42 | 3.00E−15 |
| glycylglycine | 0.37 | p < 0.0001 | 0.36 | 5.63E−12 | 0.4 | 1.76E−12 |
| docosadienoate (22:2n6) | 3.52 | p < 0.0001 | 3.9 | 1.23E−11 | 3.49 | p < 0.0001 |
| docosatrienoate (22:3n3) | 2.63 | p < 0.0001 | 2.3 | 2.65E−07 | 2.93 | p < 0.0001 |
| myristoleate (14:1n5) | 0.7 | p < 0.0001 | 0.77 | 0.0001 | 0.69 | 2.20E−10 |
| 1-linoleoylglycerophosphoethanolamine | 0.12 | p < 0.0001 | 0.11 | 4.40E−14 | 0.14 | p < 0.0001 |
| gamma-tocopherol | 5.03 | p < 0.0001 | 5.62 | 2.69E−11 | 4.85 | 1.44E−13 |
| glutamate, gamma-methyl ester | 0.43 | p < 0.0001 | 0.36 | 1.67E−07 | 0.5 | 2.55E−08 |
| 10-nonadecenoate (19:1n9) | 2.23 | p < 0.0001 | 2.26 | 2.13E−08 | 2.2 | 4.00E−15 |
| 1-arachidonoylglycerophosphoinositol | 0.54 | p < 0.0001 | 0.53 | 2.39E−07 | 0.57 | 3.97E−13 |
| valerylcarnitine | 0.55 | p < 0.0001 | 0.37 | 1.56E−10 | 0.68 | 1.06E−05 |
| laurylcarnitine | 2.73 | p < 0.0001 | 2.6 | 2.89E−07 | 2.87 | 1.97E−11 |
| 1-palmitoleoylglycerophosphoethanolamine | 0.08 | p < 0.0001 | 0.06 | 5.70E−14 | 0.09 | p < 0.0001 |
| adenosine 3′-monophosphate (3′-AMP) | 0.48 | p < 0.0001 | 0.42 | 2.17E−06 | 0.5 | 1.18E−12 |
| cysteine-glutathione disulfide | 6.25 | p < 0.0001 | 3.14 | 1.34E−07 | 7.96 | 1.39E−13 |
| maltopentaose | 4.44 | p < 0.0001 | 4.9 | 1.58E−06 | 3.84 | 2.09E−10 |
| 1-arachidonoylglycerophosphoethanolamine | 0.42 | p < 0.0001 | 0.4 | 3.49E−10 | 0.45 | p < 0.0001 |
| VGAHAGEYGAEALER | 4.98 | p < 0.0001 | 6.75 | 1.21E−08 | 4.5 | 1.75E−07 |
| 1-myristoylglycerophosphoethanolamine | 0.15 | p < 0.0001 | 0.11 | 3.62E−10 | 0.18 | 1.00E−14 |
| 2-linoleoylglycerophosphoethanolamine | 0.36 | p < 0.0001 | 0.33 | 2.45E−07 | 0.42 | 6.47E−11 |
| 7-alpha-hydroxy-3-oxo-4-cholestenoate | 4.08 | p < 0.0001 | 3.85 | 2.86E−10 | 4.35 | 3.00E−15 |
| (7-Hoca) | | | | | | |
| 5-HETE | 0.22 | p < 0.0001 | 0.25 | 1.65E−07 | 0.2 | p < 0.0001 |
| 1-pentadecanoylglycerophosphocholine | 0.28 | p < 0.0001 | 0.15 | 1.79E−11 | 0.38 | 5.41E−07 |
| 1-heptadecanoylglycerophosphoethanolamine | 0.04 | p < 0.0001 | 0.03 | p < 0.0001 | 0.06 | p < 0.0001 |
| glycerophosphoethanolamine | 0.41 | p < 0.0001 | 0.34 | 1.97E−07 | 0.46 | 7.12E−08 |
| docosapentaenoate (n6 DPA; 22:5n6) | 0.54 | p < 0.0001 | 0.45 | 2.88E−07 | 0.59 | 2.98E−09 |
| 5-oxoETE | 0.25 | p < 0.0001 | 0.27 | 2.93E−10 | 0.24 | 1.00E−15 |
| 3-hydroxyhippurate | 0.11 | p < 0.0001 | 0.08 | 1.06E−07 | 0.13 | p < 0.0001 |
| phenylalanylserine | 4.43 | p < 0.0001 | 4.2 | 1.18E−11 | 4.36 | p < 0.0001 |
| histidylleucine | 3.07 | p < 0.0001 | 2.87 | 1.78E−06 | 3.23 | 3.80E−12 |
| prolylglycine | 0.45 | p < 0.0001 | 0.44 | 8.56E−09 | 0.47 | 1.55E−10 |
| 2-stearoylglycerophosphoethanolamine | 0.03 | p < 0.0001 | 0.02 | 1.22E−10 | 0.04 | 8.00E−15 |
| phenylalanylglycine | 2.86 | p < 0.0001 | 1.92 | 1.04E−05 | 3.33 | 2.34E−11 |
| phenylalanylalanine | 7.89 | p < 0.0001 | 7.84 | 8.04E−11 | 7.85 | p < 0.0001 |
| tyrosylvaline | 3.01 | p < 0.0001 | 3.22 | 4.02E−06 | 2.9 | 1.44E−11 |
| nervonate (24:1n9) | 3.84 | p < 0.0001 | 5.53 | 4.56E−08 | 3.6 | 3.40E−11 |
| glycylthreonine | 0.3 | p < 0.0001 | 0.26 | p < 0.0001 | 0.35 | 3.49E−11 |
| lysyltyrosine | 4.76 | p < 0.0001 | 2.47 | 2.49E−06 | 6.07 | 4.08E−11 |
| guanosine | 1.84 | 1.00E−15 | 1.75 | 0.0001 | 1.99 | 6.36E−12 |
| 6-phosphogluconate | 3.14 | 1.00E−15 | 3.29 | 2.89E−07 | 3.38 | 1.21E−09 |
| 1-heptadecanoylglycerophosphocholine | 0.26 | 1.00E−15 | 0.14 | 1.61E−09 | 0.36 | 5.31E−08 |
| beta-tocopherol | 4.38 | 1.00E−15 | 5.75 | 2.33E−07 | 4.16 | 1.99E−09 |
| Isobar: ribulose 5-phosphate, xylulose | 2.16 | 1.00E−15 | 1.62 | 0.0006 | 2.56 | 8.41E−13 |
| 5-phosphate | | | | | | |
| 3-(4-hydroxyphenyl)lactate | 1.53 | 2.00E−15 | 1.83 | 6.75E−07 | 1.47 | 4.38E−08 |
| 10-heptadecenoate (17:1n7) | 1.62 | 2.00E−15 | 1.71 | 1.89E−06 | 1.61 | 6.55E−10 |
| phenylalanylproline | 2.74 | 2.00E−15 | 2.35 | 1.28E−05 | 2.94 | 5.28E−11 |
| serylleucine | 4.27 | 3.00E−15 | 3.42 | 8.75E−05 | 4.76 | 6.70E−12 |
| phenylalanylaspartate | 3.73 | 3.00E−15 | 4.38 | 1.56E−06 | 3.58 | 6.85E−11 |
| N-methylglutamate | 0.3 | 4.00E−15 | 0.23 | 2.11E−06 | 0.33 | 1.28E−07 |
| adenosine 2′-monophosphate (2′-AMP) | 0.54 | 4.00E−15 | 0.45 | 2.69E−06 | 0.6 | 3.32E−08 |
| 1-oleoylglycerophosphocholine | 0.3 | 7.00E−15 | 0.14 | 2.43E−10 | 0.44 | 5.71E−06 |
| 1-palmitoylglycerophosphocholine | 0.35 | 8.00E−15 | 0.24 | 1.04E−08 | 0.41 | 2.44E−07 |
| arachidate (20:0) | 2.39 | 1.20E−14 | 2.6 | 2.45E−08 | 2.32 | 1.19E−07 |
| 15-methylpalmitate (isobar with 2- | 1.36 | 1.20E−14 | 1.45 | 1.61E−06 | 1.33 | 9.03E−09 |
| methylpalmitate) | | | | | | |
| N-acetylserine | 0.57 | 2.80E−14 | 0.51 | 5.11E−07 | 0.64 | 5.46E−07 |
| nicotinamide adenine dinucleotide | 0.55 | 7.60E−14 | 0.35 | 5.26E−07 | 0.78 | 6.45E−06 |
| (NAD+) | | | | | | |
| N1-Methyl-2-pyridone-5-carboxamide | 0.66 | 1.15E−13 | 0.77 | 0.0039 | 0.62 | 1.89E−09 |
| 2-palmitoleoylglycerophosphocholine | 2.81 | 1.36E−13 | 1.98 | 0.0247 | 3.47 | 1.23E−12 |
| 4-hydroxyglutamate | 6.7 | 1.39E−13 | 5.59 | 6.31E−05 | 6.38 | 1.44E−08 |
| threonylphenylalanine | 5.4 | 1.84E−13 | 3.91 | 0.0022 | 5.69 | 1.70E−11 |
| phenylalanyltyrosine | 2.9 | 1.94E−13 | 2.97 | 7.30E−05 | 2.94 | 5.60E−09 |
| cytidine 5′-monophosphate (5′-CMP) | 2.21 | 2.23E−13 | 2.44 | 2.34E−07 | 2.28 | 1.40E−09 |
| tyrosylalanine | 2.36 | 2.37E−13 | 2.09 | 0.0007 | 2.5 | 3.58E−10 |
| tyrosylphenylalanine | 2.4 | 2.61E−13 | 2.45 | 7.82E−06 | 2.37 | 1.37E−08 |
| 1-stearoylglycerol (1-monostearin) | 0.61 | 4.85E−13 | 0.58 | 1.48E−06 | 0.64 | 1.98E−06 |
| oleoylcarnitine | 2.02 | 5.01E−13 | 1.54 | 0.0008 | 2.61 | 3.04E−09 |
| aspartylleucine | 2.73 | 1.28E−12 | 2.41 | 0.0006 | 2.98 | 3.12E−10 |
| glycylphenylalanine | 2.16 | 1.34E−12 | 1.96 | 0.0002 | 2.35 | 3.40E−09 |
| N-acetylglucosamine 6-phosphate | 1.94 | 1.38E−12 | 1.63 | 0.0022 | 2.21 | 6.44E−11 |
| arginylphenylalanine | 3.98 | 1.48E−12 | 2.71 | 0.0002 | 4.55 | 3.18E−09 |
| xylitol | 0.55 | 1.72E−12 | 0.43 | 1.47E−06 | 0.66 | 2.86E−05 |
| leucylhistidine | 2.03 | 2.66E−12 | 2.06 | 0.0039 | 1.77 | 1.84E−08 |
| guanosine 5′-monophosphate (5′-GMP) | 2.93 | 2.86E−12 | 3.53 | 1.04E−06 | 2.62 | 4.70E−07 |
| cytidine-3′-monophosphate (3′-CMP) | 0.59 | 3.88E−12 | 0.56 | 1.39E−05 | 0.61 | 2.15E−06 |
| phenylalanylleucine | 4.3 | 4.50E−12 | 3.51 | 2.52E−06 | 4.67 | 1.74E−07 |
| uridine monophosphate (5′ or 3′) | 2.72 | 5.60E−12 | 3 | 2.88E−06 | 2.71 | 4.81E−07 |
| 1-myristoylglycerophosphocholine | 0.38 | 6.99E−12 | 0.2 | 1.95E−08 | 0.51 | 3.98E−05 |
| spermidine | 1.7 | 7.32E−12 | 1.84 | 6.39E−06 | 1.66 | 5.36E−07 |
| tyrosylglutamine | 2.03 | 8.13E−12 | 1.91 | 2.74E−06 | 2.08 | 5.39E−07 |
| cytidine | 0.49 | 1.21E−11 | 0.34 | 1.52E−07 | 0.57 | 4.74E−05 |
| L-urobilin | 0.29 | 1.32E−11 | 0.26 | 0.0017 | 0.33 | 7.50E−09 |
| Isobar: fructose 1,6-diphosphate, | 2.99 | 1.84E−11 | 3.14 | 3.20E−06 | 2.9 | 5.23E−06 |
| glucose 1,6-diphosphate, myo-inositol | | | | | | |
| 1,4 or 1,3-diphosphate | | | | | | |
| maltohexaose | 1.64 | 1.86E−11 | 1.91 | 0.0001 | 1.42 | 4.01E−06 |
| sphingosine | 2.58 | 2.25E−11 | 1.83 | 0.0024 | 3.11 | 1.41E−07 |
| phenylalanylphenylalanine | 2.76 | 2.39E−11 | 2.73 | 5.78E−05 | 2.86 | 7.96E−07 |
| alanylleucine | 4.55 | 3.18E−11 | 3.15 | 0.0059 | 5.23 | 4.69E−09 |
| gamma-glutamylglutamine | 4.2 | 5.55E−11 | 3.54 | 5.82E−06 | 4.52 | 0.0001 |
| serylphenyalanine | 2.74 | 6.12E−11 | 2.48 | 1.75E−05 | 2.98 | 5.21E−08 |
| citrulline | 1.4 | 6.91E−11 | 1.57 | 3.29E−06 | 1.29 | 0.0002 |
| methionylalanine | 6.38 | 8.26E−11 | 5.2 | 0.0216 | 6.48 | 7.52E−09 |
| squalene | 0.6 | 1.02E−10 | 0.62 | 1.64E−06 | 0.64 | 0.0003 |
| homoserine | 1.97 | 1.18E−10 | 1.47 | 0.0492 | 2.25 | 7.80E−11 |
| arginine | 0.7 | 1.65E−10 | 0.69 | 7.02E−05 | 0.73 | 2.54E−05 |
| undecanedioate | 1.4 | 2.13E−10 | 1.49 | 0.0004 | 1.41 | 1.40E−07 |
| 2-hydroxypalmitate | 1.83 | 2.86E−10 | 1.34 | 0.0005 | 2.13 | 6.44E−06 |
| stearidonate (18:4n3) | 1.96 | 2.92E−10 | 1.93 | 8.26E−05 | 2.07 | 4.95E−06 |
| saccharopine | 5.43 | 2.99E−10 | 4.81 | 4.47E−05 | 5.78 | 2.24E−05 |
| glutathione, oxidized (GSSG) | 31.39 | 3.57E−10 | 21.01 | 0.0366 | 32.2 | 1.53E−07 |
| leucylserine | 4.22 | 3.64E−10 | 3.06 | 0.0454 | 4.6 | 2.02E−09 |
| laurate (12:0) | 0.79 | 3.94E−10 | 0.98 | 0.3717 | 0.67 | 1.06E−11 |
| tryptophylleucine | 2.62 | 1.31E−09 | 3.15 | 0.0001 | 2.38 | 1.94E−05 |
| arginylleucine | 3.88 | 1.71E−09 | 3.2 | 0.0011 | 4.12 | 2.56E−07 |
| valylmethionine | 4.01 | 2.69E−09 | 2.49 | 0.0304 | 4.77 | 4.06E−08 |
| alanylphenylalanine | 4.1 | 2.78E−09 | 3.5 | 0.002 | 4.41 | 4.83E−08 |
| phenylalanylmethionine | 2.49 | 3.30E−09 | 2.14 | 0.0014 | 2.59 | 8.97E−06 |
| phenylalanylglutamate | 3.4 | 3.36E−09 | 2.57 | 2.84E−06 | 3.93 | 7.16E−08 |
| caprate (10:0) | 0.82 | 3.57E−09 | 0.91 | 0.068 | 0.77 | 2.25E−08 |
| pregnanediol-3-glucuronide | 0.7 | 4.21E−09 | 0.68 | 0.0018 | 0.68 | 1.94E−06 |
| stearate (18:0) | 1.29 | 5.26E−09 | 1.33 | 0.0002 | 1.27 | 3.40E−05 |
| myristoylcarnitine | 1.85 | 6.64E−09 | 1.64 | 0.0122 | 2.08 | 2.15E−07 |
| 1-palmitoleoylglycerophosphocholine | 0.42 | 9.63E−09 | 0.22 | 2.06E−07 | 0.58 | 0.0045 |
| Ac-Ser-Asp-Lys-Pro-OH | 1.57 | 1.09E−08 | 1.6 | 0.0002 | 1.6 | 2.98E−05 |
| palmitoleate (16:1n7) | 1.41 | 1.44E−08 | 1.54 | 2.61E−05 | 1.39 | 2.59E−05 |
| linolenate [alpha or gamma; (18:3n3 or 6)] | 1.64 | 1.54E−08 | 1.76 | 2.17E−05 | 1.67 | 1.12E−05 |
| methylphosphate | 0.65 | 1.63E−08 | 0.56 | 0.0004 | 0.73 | 0.0003 |
| sphinganine | 2.21 | 1.99E−08 | 1.63 | 0.0569 | 2.6 | 5.63E−07 |
| palmitoylcarnitine | 1.54 | 2.31E−08 | 1.19 | 0.0332 | 1.89 | 3.08E−06 |
| 1-docosahexaenoylglycerophosphocholine | 0.54 | 2.97E−08 | 0.32 | 7.39E−10 | 0.65 | 0.007 |
| 2-stearoylglycerophosphocholine | 0.3 | 3.84E−08 | 0.15 | 4.75E−07 | 0.46 | 0.0036 |
| isoleucyltyrosine | 3.86 | 4.04E−08 | 2.75 | 0.1293 | 4.39 | 4.97E−08 |
| 1-stearoylglycerophosphocholine | 0.38 | 4.60E−08 | 0.21 | 1.37E−06 | 0.5 | 0.0012 |
| ophthalmate | 1.74 | 4.76E−08 | 1.22 | 0.1967 | 2.07 | 7.95E−07 |
| tyrosylleucine | 3.93 | 6.12E−08 | 3.54 | 0.0037 | 4.15 | 3.11E−07 |
| cinnamoylglycine | 0.75 | 6.45E−08 | 0.75 | 0.0158 | 0.75 | 1.04E−05 |
| phosphate | 0.8 | 7.35E−08 | 0.77 | 0.0016 | 0.84 | 0.001 |
| histamine | 2.57 | 9.15E−08 | 2.99 | 0.0011 | 2.32 | 0.0009 |
| trans-4-hydroxyproline | 0.82 | 1.01E−07 | 0.58 | 0.002 | 0.92 | 5.28E−05 |
| 3′-dephosphocoenzyme A | 0.53 | 1.25E−07 | 0.46 | 0.0003 | 0.63 | 0.0018 |
| caproate (6:0) | 0.82 | 1.61E−07 | 0.93 | 0.4299 | 0.75 | 2.64E−08 |
| cysteinylglycine | 6.85 | 1.75E−07 | 1.95 | 0.0866 | 9.79 | 8.35E−06 |
| aspartyltryptophan | 0.75 | 2.12E−07 | 0.6 | 5.37E−07 | 0.88 | 0.0412 |
| cytosine-2′,3′-cyclic monophosphate | 0.84 | 2.21E−07 | 0.57 | 1.31E−08 | 1 | 0.0461 |
| aspartate-glutamate | 0.84 | 2.34E−07 | 0.66 | 5.97E−06 | 0.98 | 0.0216 |
| nicotinamide ribonucleotide (NMN) | 0.52 | 3.22E−07 | 0.39 | 0.0005 | 0.68 | 0.0029 |
| gamma-glutamylcysteine | 2.72 | 3.44E−07 | 2.54 | 0.0384 | 2.9 | 1.32E−06 |
| pelargonate (9:0) | 0.88 | 5.72E−07 | 1.01 | 0.5819 | 0.79 | 3.33E−08 |
| valyltryptophan | 3.45 | 8.20E−07 | 2.77 | 0.0094 | 4.07 | 4.47E−06 |
| inosine | 1.27 | 8.34E−07 | 1.13 | 0.116 | 1.41 | 3.62E−08 |
| 2-myristoylglycerophosphocholine | 1.72 | 8.48E−07 | 1.5 | 0.1114 | 1.83 | 2.33E−05 |
| methionylglycine | 2.49 | 8.80E−07 | 1.58 | 0.3241 | 2.85 | 5.56E−07 |
| threonylleucine | 3.1 | 8.91E−07 | 2.21 | 0.0363 | 3.53 | 1.70E−06 |
| linoleate (18:2n6) | 1.34 | 1.35E−06 | 1.37 | 0.0004 | 1.34 | 0.0002 |
| histidylphenylalanine | 2.41 | 2.47E−06 | 2.49 | 0.0165 | 2.47 | 0.0001 |
| tyrosylglycine | 1.37 | 2.93E−06 | 1.45 | 0.0487 | 1.37 | 7.88E−06 |
| sorbitol 6-phosphate | 2.19 | 3.11E−06 | 2.14 | 0.1707 | 2.4 | 3.53E−06 |
| isoleucylglycine | 0.8 | 6.58E−06 | 0.74 | 3.00E−06 | 0.88 | 0.1275 |
| alanyltyrosine | 2.35 | 7.20E−06 | 2.24 | 0.0003 | 2.49 | 0.0002 |
| imidazole propionate | 0.87 | 8.19E−06 | 0.87 | 0.0702 | 0.86 | 4.55E−05 |
| methionylleucine | 3.35 | 8.35E−06 | 2.39 | 0.1661 | 3.55 | 9.16E−05 |
| ribulose | 1.62 | 8.82E−06 | 1.2 | 0.1179 | 1.88 | 1.23E−05 |
| tyrosylhistidine | 1.81 | 9.40E−06 | 2.03 | 4.04E−05 | 1.81 | 0.0004 |
| 3-phosphoglycerate | 0.59 | 9.94E−06 | 0.79 | 0.3998 | 0.52 | 7.36E−05 |
| phenylalanylvaline | 2.41 | 1.13E−05 | 2.21 | 0.0737 | 2.49 | 1.90E−05 |
| 2-oleoylglycerol (2-monoolein) | 2.61 | 1.64E−05 | 2.4 | 0.0676 | 3.21 | 2.07E−05 |
| leucylleucine | 3.55 | 1.75E−05 | 2.76 | 0.0361 | 3.99 | 2.66E−05 |
| leucylalanine | 2.54 | 1.76E−05 | 1.86 | 0.2007 | 2.86 | 5.92E−05 |
| glycyltyrosine | 1.48 | 1.81E−05 | 1.47 | 0.0065 | 1.55 | 6.69E−05 |
| heme | 2.6 | 1.97E−05 | 11.64 | 8.19E−05 | 1.49 | 0.0552 |
| deoxycarnitine | 1.27 | 2.02E−05 | 1.15 | 0.3199 | 1.37 | 6.53E−06 |
| valylleucine | 4.02 | 2.23E−05 | 2.16 | 0.0923 | 5.08 | 0.0001 |
| butyrylcarnitine | 1.47 | 2.59E−05 | 1.39 | 0.5491 | 1.66 | 1.19E−07 |
| arginyltyrosine | 2.11 | 2.93E−05 | 2.2 | 0.0967 | 2.07 | 0.0006 |
| leucylglutamate | 2.74 | 3.09E−05 | 2.13 | 0.1254 | 3.12 | 4.94E−05 |
| valylphenylalanine | 3.62 | 3.19E−05 | 2.2 | 0.1674 | 4.31 | 1.52E−05 |
| sedoheptulose-7-phosphate | 1.52 | 4.23E−05 | 0.94 | 0.9353 | 1.94 | 1.69E−06 |
| methionylasparagine | 1.94 | 4.60E−05 | 2.26 | 0.0059 | 1.87 | 0.0031 |
| spermine | 1.17 | 4.63E−05 | 4.94 | 0.0048 | 0.97 | 0.0005 |
| histidyltryptophan | 1.69 | 5.94E−05 | 1.59 | 0.0565 | 1.77 | 0.0003 |
| lysylleucine | 2.48 | 6.35E−05 | 1.75 | 0.6591 | 2.91 | 1.55E−06 |
| pentadecanoate (15:0) | 1.3 | 6.59E−05 | 1.34 | 0.0075 | 1.35 | 0.0001 |
| cis-vaccenate (18:1n7) | 1.57 | 6.63E−05 | 1.51 | 0.098 | 1.66 | 1.02E−05 |
| caprylate (8:0) | 0.86 | 6.95E−05 | 1.05 | 0.7927 | 0.76 | 4.65E−06 |
| 5-methyluridine (ribothymidine) | 0.81 | 7.09E−05 | 0.85 | 0.0057 | 0.78 | 0.0069 |
| histidyltyrosine | 2.03 | 7.44E−05 | 3.37 | 0.0503 | 1.7 | 0.0015 |
| alanylglutamate | 2.05 | 8.45E−05 | 1.43 | 0.3645 | 2.27 | 2.80E−06 |
| 2-linoleoylglycerol (2-monolinolein) | 2.25 | 8.78E−05 | 2.61 | 0.0026 | 2.18 | 0.0049 |
| histidylmethionine | 2.23 | 9.00E−05 | 2.68 | 0.023 | 2.23 | 0.0008 |
| bilirubin (Z,Z) | 1.5 | 0.0001 | 1.4 | 0.0046 | 1.17 | 0.0373 |
| methionylglutamate | 1.99 | 0.0001 | 1.88 | 0.091 | 2.14 | 0.0014 |
| 1-palmitoylglycerol (1-monopalmitin) | 0.78 | 0.0002 | 0.65 | 0.0028 | 0.89 | 0.1082 |
| 3-hydroxyoctanoate | 0.8 | 0.0002 | 0.78 | 0.0118 | 0.79 | 0.0078 |
| glycylisoleucine | 0.83 | 0.0002 | 0.67 | 7.07E−05 | 0.97 | 0.3598 |
| isoleucylmethionine | 3.9 | 0.0002 | 2.39 | 0.8164 | 4.65 | 2.61E−06 |
| S-methylcysteine | 0.81 | 0.0002 | 0.8 | 0.0405 | 0.87 | 0.0489 |
| valylglycine | 0.87 | 0.0002 | 0.73 | 2.17E−05 | 1 | 0.3709 |
| tyrosyltyrosine | 2.04 | 0.0002 | 1.87 | 0.1295 | 2.16 | 0.0011 |
| alanyltryptophan | 1.72 | 0.0002 | 2.45 | 6.65E−05 | 1.46 | 0.0587 |
| oleate (18:1n9) | 1.49 | 0.0003 | 1.47 | 0.0601 | 1.55 | 0.0003 |
| 2-ethylhexanoate | 0.93 | 0.0003 | 1.23 | 0.9113 | 0.71 | 1.57E−06 |
| 2-docosapentaenoylglycerophosphoethanolamine | 1.71 | 0.0003 | 1.35 | 0.4746 | 1.82 | 0.0051 |
| thymidine | 0.75 | 0.0003 | 0.64 | 0.0015 | 0.79 | 0.0341 |
| 1-oleoylglycerol (1-monoolein) | 1.65 | 0.0004 | 1.41 | 0.2749 | 1.79 | 0.0002 |
| adenosine 5′-monophosphate (AMP) | 1.9 | 0.0005 | 2.28 | 0.0005 | 1.82 | 0.0135 |
| choline phosphate | 1.31 | 0.0005 | 1.47 | 0.0003 | 1.25 | 0.0482 |
| 4-hydroxybutyrate (GHB) | 3.12 | 0.0005 | 1.92 | 0.6215 | 3.69 | 1.70E−06 |
| 2-oleoylglycerophosphoserine | 0.96 | 0.0005 | 0.93 | 0.0122 | 1.05 | 0.2395 |
| leucylglycine | 2.53 | 0.0005 | 1.65 | 0.5448 | 2.95 | 0.0002 |
| valyltyrosine | 3.12 | 0.0005 | 2.25 | 0.6048 | 3.51 | 8.19E−05 |
| valylserine | 1.96 | 0.0005 | 1.08 | 0.83 | 2.5 | 3.84E−05 |
| valylarginine | 1.72 | 0.0005 | 1.96 | 0.0482 | 1.65 | 0.003 |
| nicotinamide | 0.86 | 0.0008 | 0.88 | 0.0674 | 0.9 | 0.0856 |
| leucylmethionine | 1.09 | 0.0008 | 0.75 | 0.0001 | 1.36 | 0.338 |
| isoleucyltryptophan | 3.04 | 0.0008 | 1.44 | 0.5864 | 3.93 | 8.60E−06 |
| valylhistidine | 0.82 | 0.0009 | 0.54 | 0.0003 | 1.04 | 0.2933 |
| arginylmethionine | 1.8 | 0.0009 | 2.24 | 0.0454 | 1.62 | 0.0155 |
| 2-arachidonoylglycerophosphoethanolamine | 0.88 | 0.0011 | 0.81 | 0.0182 | 0.99 | 0.2724 |
| alanylmethionine | 2.32 | 0.0012 | 1.86 | 0.1669 | 2.51 | 0.0023 |
| threonylvaline | 1.79 | 0.0012 | 1.84 | 0.1523 | 1.71 | 0.0085 |
| 6-keto prostaglandin F1alpha | 0.65 | 0.0015 | 0.53 | 0.0263 | 0.72 | 0.0468 |
| leucyltyrosine | 1.97 | 0.0015 | 1.76 | 0.7723 | 1.92 | 0.0036 |
| 7-beta-hydroxycholesterol | 1.71 | 0.0016 | 1.27 | 0.3887 | 2.01 | 0.0043 |
| glycylmethionine | 1.7 | 0.0016 | 1.45 | 0.3622 | 1.86 | 0.0006 |
| pyrophosphate (PPi) | 0.72 | 0.0018 | 0.64 | 0.0162 | 0.7 | 0.0274 |
| aspartylphenylalanine | 1.82 | 0.0019 | 1.45 | 0.6813 | 2.03 | 4.59E−05 |
| 16-hydroxypalmitate | 0.74 | 0.0019 | 0.83 | 0.0121 | 0.66 | 0.0316 |
| 1-linoleoylglycerophosphocholine | 0.64 | 0.0025 | 0.37 | 0.0001 | 0.9 | 0.5971 |
| valylglutamate | 1.84 | 0.003 | 1.43 | 0.8909 | 2.1 | 4.15E−05 |
| cystine | 1.58 | 0.003 | 1.89 | 0.0601 | 1.46 | 0.0657 |
| phosphoethanolamine | 0.92 | 0.0032 | 0.92 | 0.0974 | 0.95 | 0.0686 |
| N-acetyltryptophan | 0.1 | 0.0035 | 0.09 | 0.1115 | 0.1 | 0.023 |
| 3-hydroxydecanoate | 0.76 | 0.0036 | 0.77 | 0.0443 | 0.77 | 0.0623 |
| betaine | 0.79 | 0.0036 | 0.72 | 0.19 | 0.85 | 0.0241 |
| leucylasparagine | 2.07 | 0.0036 | 1.6 | 0.9498 | 2.27 | 0.0012 |
| cytidine 5′-diphosphocholine | 1.85 | 0.0037 | 1.52 | 0.6134 | 1.98 | 0.0014 |
| leucylphenylalanine | 2.15 | 0.0038 | 1.59 | 0.9033 | 2.37 | 0.0008 |
| tryptophylglutamate | 1.56 | 0.0042 | 1.62 | 0.2478 | 1.58 | 0.0029 |
| 2-phosphoglycerate | 0.61 | 0.0054 | 0.73 | 0.1842 | 0.54 | 0.0129 |
| 6′-sialyllactose | 2.62 | 0.007 | 2.49 | 0.1936 | 2.85 | 0.0038 |
| margarate (17:0) | 1.15 | 0.0076 | 1.16 | 0.0824 | 1.14 | 0.0527 |
| glycerate | 0.85 | 0.0076 | 0.86 | 0.0664 | 0.86 | 0.0993 |
| isoleucylhistidine | 0.7 | 0.0077 | 0.7 | 0.1031 | 0.81 | 0.3691 |
| alpha-glutamyltyrosine | 2.04 | 0.0079 | 1.68 | 0.78 | 2.28 | 0.0011 |
| tryptophylasparagine | 2.15 | 0.0083 | 1.7 | 0.4846 | 2.34 | 0.0006 |
| arginylvaline | 1.3 | 0.0099 | 1.47 | 0.1562 | 1.23 | 0.0646 |
| adenylosuccinate | 0.81 | 0.0103 | 0.6 | 0.002 | 1.11 | 0.7343 |
| myristate (14:0) | 0.94 | 0.0107 | 1.05 | 0.5054 | 0.88 | 0.0017 |
| lysylmethionine | 1.28 | 0.0107 | 1.46 | 0.8904 | 1.22 | 0.0035 |
| 1-linoleoylglycerol (1-monolinolein) | 1.67 | 0.0125 | 1.6 | 0.2315 | 1.67 | 0.0181 |
| 1-arachidonylglycerol | 0.74 | 0.0132 | 0.86 | 0.6146 | 0.72 | 0.0457 |
| guanine | 0.89 | 0.0136 | 0.48 | 0.5964 | 1.15 | 0.0572 |
| glycerol 2-phosphate | 1.59 | 0.0137 | 1.4 | 0.2948 | 1.79 | 0.0048 |
| 2′-deoxyinosine | 1.32 | 0.0144 | 1.05 | 0.7128 | 1.42 | 0.0052 |
| palmitate (16:0) | 1.13 | 0.0168 | 1.18 | 0.0478 | 1.11 | 0.1342 |
| prostaglandin A2 | 0.65 | 0.0188 | 0.51 | 0.112 | 0.71 | 0.1511 |
| isoleucylarginine | 1.02 | 0.0194 | 1.05 | 0.002 | 1.02 | 0.9057 |
| phenylalanyltryptophan | 1.52 | 0.0203 | 1.53 | 0.5818 | 1.47 | 0.0491 |
| homocysteine | 1 | 0.0228 | 0.42 | 0.0004 | 1.49 | 0.4194 |
| 1,3-dihydroxyacetone | 1.37 | 0.024 | 1.03 | 0.7914 | 1.48 | 0.0102 |
| 1-arachidonoylglycerophosphocholine | 0.8 | 0.0269 | 0.49 | 0.0002 | 1.05 | 0.9462 |
| aspartylvaline | 1.4 | 0.0269 | 0.72 | 0.0008 | 1.74 | 0.6929 |
| 2-oleoylglycerophosphocholine | 0.85 | 0.0275 | 0.48 | 0.0008 | 1.16 | 0.9341 |
| threonylmethionine | 1.81 | 0.0281 | 1.3 | 0.7264 | 2.07 | 0.0025 |
| dihydrocholesterol | 1.46 | 0.0314 | 1.12 | 0.2523 | 1.9 | 0.0001 |
| valylasparagine | 1.63 | 0.0314 | 0.84 | 0.1212 | 2.13 | 0.0015 |
| uridine | 0.89 | 0.0331 | 0.8 | 0.0181 | 0.96 | 0.5118 |
| 2-palmitoylglycerophosphocholine | 0.66 | 0.0362 | 0.37 | 0.0007 | 0.89 | 0.7683 |
| 7-alpha-hydroxycholesterol | 2.52 | 0.0367 | 1.53 | 0.9998 | 2.73 | 0.0665 |
| cholesterol | 1.16 | 0.0369 | 1.07 | 0.3459 | 1.26 | 0.0146 |
| isoleucylisoleucine | 2.26 | 0.0383 | 1.89 | 0.8332 | 2.43 | 0.0087 |
| alpha-glutamyltryptophan | 1.8 | 0.0389 | 1.36 | 0.6571 | 2.05 | 0.0044 |
| isoleucylserine | 1.94 | 0.0408 | 1.38 | 0.8156 | 2.28 | 0.0046 |
| bilirubin (E,E) | 1.23 | 0.0433 | 1.17 | 0.0457 | 1.02 | 0.7542 |
| stearoylcarnitine | 1.2 | 0.0435 | 0.95 | 0.9679 | 1.48 | 0.0366 |
| 1,2-propanediol | 0.87 | 0.0507 | 0.95 | 0.946 | 0.85 | 0.0454 |
| 2-docosahexaenoylglycerophosphocholine | 0.87 | 0.0575 | 0.58 | 0.0069 | 1.04 | 0.6503 |
| prostaglandin E2 | 0.53 | 0.0624 | 0.29 | 0.2867 | 0.83 | 0.2277 |
| methionylaspartate | 1.7 | 0.0633 | 1.66 | 0.3022 | 1.88 | 0.0767 |
| isoleucylalanine | 2.01 | 0.0751 | 1.44 | 0.5482 | 2.32 | 0.0015 |
| N-acetylglucosamine | 0.66 | 0.0835 | 0.57 | 0.0957 | 0.68 | 0.2922 |
| triethyleneglycol | 0.9 | 0.0988 | 0.82 | 0.0476 | 1.06 | 0.696 |
| threonylglutamate | 1.11 | 0.0999 | 0.88 | 0.0274 | 1.25 | 0.882 |
| valylalanine | 1.78 | 0.1209 | 1.36 | 0.4229 | 1.99 | 0.0049 |
| hypotaurine | 1.69 | 0.1214 | 1.87 | 0.0574 | 1.77 | 0.144 |
| 2′-deoxyadenosine 3′-monophosphate | 1.21 | 0.1295 | 1.05 | 0.9603 | 1.33 | 0.0266 |
| palmitoyl sphingomyelin | 0.92 | 0.1296 | 0.86 | 0.1301 | 0.99 | 0.7402 |
| argininosuccinate | 0.53 | 0.1327 | 0.47 | 0.0623 | 0.56 | 0.6963 |
| adrenate (22:4n6) | 1.12 | 0.1383 | 0.99 | 0.7539 | 1.21 | 0.0211 |
| alanylalanine | 1.1 | 0.1551 | 1.05 | 0.0105 | 1.15 | 0.8715 |
| 2′-deoxycytidine 3′-monophosphate | 1.21 | 0.1915 | 1.01 | 0.933 | 1.2 | 0.6439 |
| S-adenosylmethionine (SAM) | 1.24 | 0.196 | 0.83 | 0.0027 | 1.48 | 0.0004 |
| alanylthreonine | 1.66 | 0.201 | 1.74 | 0.5377 | 1.72 | 0.014 |
| tyrosyllysine | 1.62 | 0.2136 | 0.81 | 0.1455 | 2.33 | 0.0318 |
| valylglutamine | 1.66 | 0.2152 | 1.11 | 0.1806 | 2.01 | 0.0048 |
| phytosphingosine | 0.82 | 0.2359 | 0.69 | 0.1964 | 0.96 | 0.8095 |
| cortisol | 0.74 | 0.2361 | 0.51 | 0.8553 | 0.95 | 0.5266 |
| valyllysine | 1.12 | 0.2369 | 0.74 | 0.0346 | 1.37 | 0.5939 |
| serylvaline | 1.59 | 0.2378 | 1.29 | 0.3069 | 1.74 | 0.0141 |
| leucylarginine | 1.56 | 0.2687 | 1.43 | 0.7131 | 1.59 | 0.0396 |
| 2-arachidonoylglycerophosphocholine | 1.3 | 0.2775 | 0.73 | 0.0671 | 1.79 | 0.019 |
| glycyllysine | 1.13 | 0.282 | 1.14 | 0.6421 | 1.25 | 0.266 |
| galactose | 1.5 | 0.2857 | 1.4 | 0.6402 | 1.5 | 0.0284 |
| valylvaline | 1.92 | 0.3058 | 1.22 | 0.2967 | 2.3 | 0.0219 |
| nicotinamide adenine dinucleotide | 1.45 | 0.3061 | 1.57 | 0.5098 | 1.53 | 0.3233 |
| reduced (NADH) | | | | | | |
| agmatine | 1.53 | 0.3279 | 0.83 | 0.2243 | 2.31 | 0.0026 |
| leucyltryptophan | 1.18 | 0.3339 | 1.06 | 0.3349 | 1.24 | 0.0976 |
| ribose | 1.19 | 0.3602 | 0.72 | 0.0034 | 1.53 | 0.0555 |
| alpha-glutamylglutamate | 1.55 | 0.3695 | 1.17 | 0.5033 | 1.8 | 0.075 |
| prolylmethionine | 1.78 | 0.3832 | 1.39 | 0.1804 | 2.09 | 0.0024 |
| 2-palmitoylglycerol (2-monopalmitin) | 1 | 0.4149 | 0.87 | 0.0578 | 1.15 | 0.2072 |
| dodecanedioate | 0.92 | 0.4214 | 1.03 | 0.8457 | 0.82 | 0.0947 |
| valylisoleucine | 2.09 | 0.4309 | 1.38 | 0.1845 | 2.43 | 0.0355 |
| 2′-deoxyguanosine | 1.18 | 0.4593 | 0.93 | 0.1993 | 1.35 | 0.0602 |
| 2-docosapentaenoylglycerophosphocholine | 1.1 | 0.4792 | 0.63 | 0.0546 | 1.44 | 0.0556 |
| glycylleucine | 1.13 | 0.486 | 1.12 | 0.0573 | 1.2 | 0.2792 |
| serylisoleucine | 1.25 | 0.5075 | 1.23 | 0.1074 | 1.33 | 0.2853 |
| N-acetylornithine | 1.11 | 0.5223 | 1.2 | 0.2014 | 1.13 | 0.4737 |
| isoleucylvaline | 1.8 | 0.523 | 1.21 | 0.009 | 2.13 | 0.0923 |
| arabonate | 1.07 | 0.5252 | 1.21 | 0.0977 | 1.04 | 0.9216 |
| ornithine | 1.17 | 0.5853 | 1.58 | 0.0488 | 1.07 | 0.2307 |
| glycyltryptophan | 1.4 | 0.5951 | 1.22 | 0.3179 | 1.6 | 0.059 |
| testosterone | 1.01 | 0.6287 | 1.27 | 0.0247 | 0.89 | 0.3475 |
| methionylphenylalanine | 1.47 | 0.6522 | 1.23 | 0.0263 | 1.3 | 0.236 |
| alanylglycine | 1.26 | 0.7033 | 0.96 | 0.1068 | 1.45 | 0.0723 |
| alanylvaline | 1.4 | 0.7425 | 1.21 | 0.1474 | 1.54 | 0.1896 |
| isoleucylphenylalanine | 2.97 | 0.7426 | 1.88 | 0.4284 | 3.45 | 0.1202 |
| docosapentaenoate (n3 DPA; 22:5n3) | 1.09 | 0.7743 | 1.03 | 0.6054 | 1.14 | 0.6734 |
| valylaspartate | 1.38 | 0.7778 | 1.05 | 0.0819 | 1.63 | 0.1175 |
| 2-linoleoylglycerophosphocholine | 1.11 | 0.8078 | 0.66 | 0.0131 | 1.58 | 0.0463 |
| piperine | 1.08 | 0.8111 | 1.1 | 0.9512 | 1.05 | 0.8957 |
| 13-HODE + 9-HODE | 1.15 | 0.8212 | 1.3 | 0.9076 | 1.04 | 0.9013 |
| alanylisoleucine | 1.53 | 0.8533 | 1.14 | 0.0337 | 1.8 | 0.0789 |
| lysyllysine | 1.17 | 0.8843 | 1 | 0.1283 | 1.25 | 0.175 |
| dihomo-linolenate (20:3n3 or n6) | 1.08 | 0.9478 | 0.86 | 0.0567 | 1.25 | 0.0966 |
| 2-eicosatrienoylglycerophosphocholine | 1.21 | 0.9714 | 0.55 | 0.0036 | 1.87 | 0.0338 |
| phenylalanylarginine | 1.21 | 0.9854 | 1.7 | 0.2294 | 1.05 | 0.627 |
| nicotinamide riboside | 1.18 | 0.9877 | 0.82 | 0.1453 | 1.65 | 0.0561 |
| 2-docosahexaenoylglycerophosphoethanolamine | 1.1 | 0.9879 | 0.89 | 0.2814 | 1.18 | 0.8106 |
| isoleucylglutamate | 1.3 | 0.9945 | 0.94 | 0.0357 | 1.53 | 0.0811 |
| creatinine | 0.33 | p < 0.0001 | 0.38 | 1.00E−15 | 0.32 | p < 0.0001 |
| N-acetylneuraminate | 2.45 | p < 0.0001 | 3.09 | 9.66E−12 | 2.34 | 6.31E−13 |
| 4-hydroxyhippurate | 0.09 | p < 0.0001 | 0.16 | 9.72E−12 | 0.08 | p < 0.0001 |
| malonylcarnitine | 0.36 | p < 0.0001 | 0.27 | 9.78E−11 | 0.4 | p < 0.0001 |
| 3-methylglutarylcarnitine (C6) | 0.51 | p < 0.0001 | 0.72 | 3.19E−10 | 0.25 | p < 0.0001 |
| tryptophan betaine | 2.84 | p < 0.0001 | 2.47 | 7.85E−08 | 3.21 | 2.00E−14 |
| 2-hydroxyglutarate | 6.14 | p < 0.0001 | 4.68 | 0.0002 | 7.38 | p < 0.0001 |
| chiro-inositol | 0.36 | 4.19E−11 | 0.42 | 0.0001 | 0.37 | 1.30E−05 |
| glycolithocholate sulfate | 0.69 | 2.99E−06 | 0.91 | 0.6539 | 0.59 | 6.79E−07 |
| pregnen-diol disulfate | 0.65 | 2.93E−05 | 0.92 | 0.1813 | 0.54 | 2.15E−05 |
| C-glycosyltryptophan | 0.8 | 0.0004 | 0.96 | 0.3785 | 0.74 | 0.0021 |
| glycocholenate sulfate | 0.88 | 0.0024 | 0.88 | 0.0484 | 0.86 | 0.0125 |
| succinylcarnitine | 0.91 | 0.0029 | 0.91 | 0.0796 | 0.93 | 0.0681 |
| 4-androsten-3beta,17beta-diol disulfate 1 | 0.82 | 0.0488 | 1.11 | 0.5082 | 0.7 | 0.0234 |
| glycerol | 1 | 0.0677 | 0.95 | 0.1488 | 1.06 | 0.7738 |
| 1,5-anhydroglucitol (1,5-AG) | 0.98 | 0.1785 | 1.07 | 0.2849 | 0.94 | 0.0714 |
| 4-methyl-2-oxopentanoate | 1.1 | 0.3792 | 1.04 | 0.9335 | 1.13 | 0.3022 |
| glutarate (pentanedioate) | 1.2 | 0.6189 | 0.92 | 0.1615 | 1.31 | 0.7364 |
| 2-hydroxybutyrate (AHB) | 1.05 | 0.7168 | 1.17 | 0.0306 | 0.96 | 0.2883 |
| tryptophan | 0.31 | p < 0.0001 | 0.29 | 5.90E−14 | 0.33 | p < 0.0001 |
| beta-alanine | 4.27 | p < 0.0001 | 5.68 | 2.32E−13 | 4.09 | 1.42E−10 |
| glutamate | 1.5 | p < 0.0001 | 1.45 | 2.78E−06 | 1.57 | 1.53E−13 |
| histidine | 0.49 | p < 0.0001 | 0.51 | 1.62E−09 | 0.5 | 9.00E−15 |
| leucine | 0.59 | p < 0.0001 | 0.55 | 1.11E−10 | 0.62 | 4.23E−10 |
| phenylalanine | 0.59 | p < 0.0001 | 0.55 | 6.65E−10 | 0.63 | 1.77E−09 |
| 4-hydroxyphenylacetate | 0.31 | p < 0.0001 | 0.32 | 4.92E−11 | 0.31 | p < 0.0001 |
| fructose | 4.9 | p < 0.0001 | 3.72 | 0.0001 | 5.32 | p < 0.0001 |
| gluconate | 0.3 | p < 0.0001 | 0.33 | 8.03E−09 | 0.3 | 6.31E−12 |
| trans-urocanate | 0.5 | p < 0.0001 | 0.59 | 1.15E−05 | 0.45 | p < 0.0001 |
| isoleucine | 0.55 | p < 0.0001 | 0.5 | 1.50E−11 | 0.59 | 8.50E−12 |
| threonine | 0.39 | p < 0.0001 | 0.36 | 4.23E−10 | 0.42 | 1.90E−11 |
| tyrosine | 0.51 | p < 0.0001 | 0.47 | 8.54E−12 | 0.54 | 1.86E−13 |
| methionine | 0.49 | p < 0.0001 | 0.44 | 2.98E−12 | 0.52 | 1.21E−12 |
| malate | 0.48 | p < 0.0001 | 0.46 | 1.65E−07 | 0.52 | 1.02E−09 |
| gamma-aminobutyrate (GABA) | 0.26 | p < 0.0001 | 0.27 | 1.12E−08 | 0.26 | 1.05E−13 |
| pantothenate | 0.21 | p < 0.0001 | 0.21 | p < 0.0001 | 0.23 | p < 0.0001 |
| sarcosine (N-Methylglycine) | 2.78 | p < 0.0001 | 2.23 | 1.93E−08 | 2.98 | 7.13E−12 |
| 5,6-dihydrouracil | 2.51 | p < 0.0001 | 2.11 | 2.75E−05 | 2.85 | 1.96E−12 |
| citrate | 3.32 | p < 0.0001 | 14.84 | p < 0.0001 | 1.83 | 2.47E−08 |
| vanillylmandelate (VMA) | 0.09 | p < 0.0001 | 0.12 | p < 0.0001 | 0.09 | p < 0.0001 |
| fumarate | 0.29 | p < 0.0001 | 0.24 | 3.58E−13 | 0.32 | 1.00E−15 |
| serine | 0.34 | p < 0.0001 | 0.31 | 1.01E−11 | 0.36 | 4.00E−14 |
| valine | 0.54 | p < 0.0001 | 0.52 | 3.58E−10 | 0.57 | 3.58E−13 |
| cortisone | 0.27 | p < 0.0001 | 0.23 | 3.39E−07 | 0.28 | 1.05E−10 |
| riboflavin (Vitamin B2) | 0.42 | p < 0.0001 | 0.4 | 4.86E−09 | 0.45 | 1.57E−13 |
| proline | 0.5 | p < 0.0001 | 0.46 | 3.31E−13 | 0.54 | 4.90E−14 |
| hypoxanthine | 0.59 | p < 0.0001 | 0.54 | 5.24E−09 | 0.63 | 5.15E−13 |
| xanthine | 0.66 | p < 0.0001 | 0.54 | 1.00E−11 | 0.74 | 5.78E−08 |
| cis-aconitate | 2.18 | p < 0.0001 | 4.78 | 6.28E−12 | 1.48 | 2.24E−05 |
| xanthosine | 0.53 | p < 0.0001 | 0.42 | 3.31E−11 | 0.58 | 1.59E−11 |
| kynurenine | 7.89 | p < 0.0001 | 8.74 | 2.50E−14 | 7.74 | p < 0.0001 |
| mannitol | 0.26 | p < 0.0001 | 0.29 | 9.48E−07 | 0.22 | 5.68E−12 |
| glucuronate | 0.3 | p < 0.0001 | 0.25 | 6.43E−09 | 0.34 | 1.58E−13 |
| choline | 0.66 | p < 0.0001 | 0.79 | 1.22E−05 | 0.6 | p < 0.0001 |
| N1-methyladenosine | 0.28 | p < 0.0001 | 0.35 | 6.36E−13 | 0.26 | p < 0.0001 |
| 3-methylhistidine | 0.55 | p < 0.0001 | 0.63 | 3.93E−08 | 0.51 | 1.92E−11 |
| glycolate (hydroxyacetate) | 0.71 | p < 0.0001 | 0.72 | 2.73E−05 | 0.71 | 1.78E−11 |
| anserine | 0.27 | p < 0.0001 | 0.22 | 1.16E−05 | 0.34 | 2.95E−09 |
| hippurate | 0.1 | p < 0.0001 | 0.11 | p < 0.0001 | 0.09 | p < 0.0001 |
| aspartate | 0.46 | p < 0.0001 | 0.54 | 2.62E−06 | 0.45 | 1.78E−12 |
| myo-inositol | 0.32 | p < 0.0001 | 0.28 | 2.83E−10 | 0.4 | 9.50E−13 |
| glucose | 4.18 | p < 0.0001 | 3.19 | 6.35E−09 | 4.48 | p < 0.0001 |
| adipate | 0.28 | p < 0.0001 | 0.25 | 5.62E−10 | 0.34 | 1.14E−10 |
| 2-hydroxyisobutyrate | 0.41 | p < 0.0001 | 0.46 | 3.10E−09 | 0.41 | p < 0.0001 |
| citramalate | 0.19 | p < 0.0001 | 0.15 | 1.90E−14 | 0.22 | p < 0.0001 |
| N-acetylaspartate (NAA) | 0.09 | p < 0.0001 | 0.07 | p < 0.0001 | 0.11 | p < 0.0001 |
| indoleacetate | 0.2 | p < 0.0001 | 0.2 | 9.45E−13 | 0.2 | p < 0.0001 |
| pyridoxate | 0.29 | p < 0.0001 | 0.31 | 3.20E−14 | 0.27 | p < 0.0001 |
| androsterone sulfate | 0.59 | p < 0.0001 | 0.76 | 0.0007 | 0.52 | 1.94E−13 |
| N1-methylguanosine | 0.19 | p < 0.0001 | 0.18 | p < 0.0001 | 0.2 | p < 0.0001 |
| acetylcarnitine | 2.77 | p < 0.0001 | 2.62 | 1.37E−08 | 2.92 | p < 0.0001 |
| 1-methylimidazoleacetate | 0.58 | p < 0.0001 | 0.77 | 0.0024 | 0.49 | 2.00E−15 |
| scyllo-inositol | 0.23 | p < 0.0001 | 0.16 | 4.70E−14 | 0.33 | p < 0.0001 |
| trigonelline (N′-methylnicotinate) | 0.39 | p < 0.0001 | 0.33 | 4.58E−08 | 0.41 | 3.40E−14 |
| phenol sulfate | 0.51 | p < 0.0001 | 0.78 | 0.0078 | 0.44 | p < 0.0001 |
| pyroglutamine | 3.61 | p < 0.0001 | 3.18 | 1.23E−05 | 3.98 | 2.00E−15 |
| pseudouridine | 0.28 | p < 0.0001 | 0.26 | p < 0.0001 | 0.3 | p < 0.0001 |
| N-acetylglutamine | 6.41 | p < 0.0001 | 7.39 | 5.88E−11 | 6.11 | 6.76E−13 |
| isovalerylcarnitine | 0.28 | p < 0.0001 | 0.22 | 1.40E−14 | 0.33 | 1.10E−13 |
| phenylacetylglutamine | 0.1 | p < 0.0001 | 0.12 | p < 0.0001 | 0.1 | p < 0.0001 |
| pro-hydroxy-pro | 0.43 | p < 0.0001 | 0.37 | 1.44E−10 | 0.46 | p < 0.0001 |
| N2-methylguanosine | 0.26 | p < 0.0001 | 0.19 | p < 0.0001 | 0.28 | p < 0.0001 |
| N2,N2-dimethylguanosine | 0.19 | p < 0.0001 | 0.22 | p < 0.0001 | 0.17 | p < 0.0001 |
| N6-carbamoylthreonyladenosine | 0.37 | p < 0.0001 | 0.36 | p < 0.0001 | 0.37 | p < 0.0001 |
| 2-methylbutyrylcarnitine (C5) | 0.35 | p < 0.0001 | 0.28 | 6.10E−14 | 0.41 | p < 0.0001 |
| N-acetyl-aspartyl-glutamate (NAAG) | 0.18 | p < 0.0001 | 0.19 | p < 0.0001 | 0.19 | p < 0.0001 |
| threitol | 0.57 | p < 0.0001 | 0.3 | 7.22E−10 | 0.69 | 1.64E−12 |
| p-cresol sulfate | 0.55 | p < 0.0001 | 0.73 | 0.0063 | 0.49 | 1.50E−14 |
| N6-acetyllysine | 0.22 | p < 0.0001 | 0.22 | 2.00E−15 | 0.22 | p < 0.0001 |
| dimethylarginine (SDMA + ADMA) | 0.28 | p < 0.0001 | 0.31 | 6.23E−12 | 0.26 | p < 0.0001 |
| glycylproline | 1.7 | 1.00E−15 | 1.57 | 5.31E−05 | 1.84 | 3.80E−12 |
| glutarylcarnitine (C5) | 0.46 | 1.00E−15 | 0.44 | 2.09E−07 | 0.46 | 4.92E−09 |
| catechol sulfate | 0.57 | 1.20E−14 | 0.57 | 0.0001 | 0.56 | 6.36E−10 |
| glutamine | 1.37 | 1.30E−14 | 1.44 | 1.62E−07 | 1.35 | 2.32E−07 |
| isobutyrylcarnitine | 0.66 | 2.80E−14 | 0.67 | 4.59E−05 | 0.71 | 1.79E−07 |
| gamma-glutamylisoleucine | 0.52 | 3.10E−14 | 0.59 | 0.0031 | 0.47 | 6.86E−11 |
| octanoylcarnitine | 2.14 | 3.50E−14 | 1.91 | 5.49E−05 | 2.24 | 5.96E−09 |
| gulono-1,4-lactone | 0.48 | 3.90E−14 | 0.56 | 0.008 | 0.48 | 4.78E−10 |
| urate | 0.74 | 2.01E−13 | 0.89 | 0.0108 | 0.64 | 2.49E−13 |
| 2-aminoadipate | 4.63 | 3.51E−13 | 5.01 | 1.79E−08 | 4.56 | 1.64E−06 |
| guanidinoacetate | 0.46 | 4.55E−13 | 0.41 | 3.18E−05 | 0.5 | 1.81E−07 |
| quinate | 0.43 | 4.73E−13 | 0.54 | 0.0033 | 0.42 | 3.62E−08 |
| lysine | 0.64 | 1.08E−12 | 0.63 | 1.99E−05 | 0.66 | 3.82E−07 |
| 5-aminovalerate | 1.82 | 3.24E−12 | 1.42 | 0.0066 | 2.22 | 4.74E−11 |
| 3-aminoisobutyrate | 3.86 | 3.38E−12 | 4.95 | 1.21E−08 | 3.91 | 4.92E−07 |
| sorbitol | 6.4 | 3.78E−12 | 7.27 | 1.60E−05 | 6.74 | 8.12E−08 |
| S-adenosylhomocysteine (SAH) | 2.09 | 4.41E−12 | 1.44 | 0.0838 | 2.58 | 6.79E−13 |
| tartarate | 0.08 | 1.24E−11 | 0.3 | 0.0007 | 0.07 | 4.50E−08 |
| creatine | 2.09 | 5.21E−11 | 1.67 | 0.0005 | 2.57 | 9.74E−10 |
| 2-isopropylmalate | 0.58 | 8.52E−11 | 0.61 | 1.73E−05 | 0.58 | 3.15E−05 |
| gamma-glutamylphenylalanine | 0.73 | 1.58E−10 | 0.89 | 0.1345 | 0.67 | 2.95E−08 |
| N-acetylarginine | 4.49 | 1.70E−10 | 4.01 | 0.0001 | 4.89 | 1.55E−06 |
| uracil | 0.66 | 1.86E−10 | 0.63 | 1.86E−05 | 0.7 | 6.75E−05 |
| N-6-trimethyllysine | 0.63 | 2.64E−10 | 0.67 | 0.0003 | 0.62 | 1.65E−05 |
| homostachydrine | 1.57 | 2.82E−10 | 1.48 | 0.0002 | 1.6 | 2.57E−07 |
| xylulose | 1.69 | 5.34E−10 | 1.41 | 0.0047 | 1.81 | 1.30E−07 |
| xylose | 0.21 | 3.60E−09 | 0.23 | 0.0563 | 0.2 | 1.37E−07 |
| 3-indoxyl sulfate | 0.47 | 4.38E−09 | 0.69 | 0.0691 | 0.37 | 1.06E−07 |
| adenosine | 0.65 | 6.10E−09 | 0.62 | 0.0019 | 0.69 | 2.75E−05 |
| hexanoylcarnitine | 1.51 | 2.94E−08 | 1.32 | 0.1342 | 1.75 | 4.14E−09 |
| 5-oxoproline | 0.84 | 4.46E−08 | 1.3 | 0.1643 | 0.62 | 4.09E−13 |
| stachydrine | 1.3 | 9.15E−08 | 1.28 | 0.0008 | 1.32 | 0.0002 |
| alanine | 0.74 | 1.01E−07 | 0.68 | 0.0002 | 0.79 | 0.0014 |
| lactate | 1.48 | 2.22E−07 | 1.41 | 0.0103 | 1.58 | 6.17E−06 |
| N-acetylleucine | 2.03 | 8.18E−07 | 1.47 | 0.1471 | 2.44 | 3.12E−06 |
| glycerophosphorylcholine (GPC) | 1.57 | 4.83E−06 | 1.3 | 0.318 | 1.84 | 2.39E−09 |
| cholate | 0.66 | 7.93E−06 | 0.8 | 0.1036 | 0.57 | 3.43E−05 |
| N-acetylphenylalanine | 0.78 | 9.93E−06 | 0.57 | 1.26E−05 | 1.05 | 0.1404 |
| succinate | 1.97 | 1.11E−05 | 1.45 | 0.2597 | 2.31 | 5.95E−06 |
| mannose | 2.1 | 1.60E−05 | 1.25 | 0.9842 | 2.56 | 9.59E−07 |
| benzoate | 0.87 | 2.88E−05 | 1.14 | 0.8585 | 0.7 | 1.36E−07 |
| N-acetylasparagine | 2.25 | 5.84E−05 | 2.11 | 0.0279 | 2.38 | 0.0017 |
| propionylcarnitine | 0.88 | 7.81E−05 | 0.74 | 0.0007 | 0.97 | 0.0755 |
| 2-hydroxyhippurate (salicylurate) | 0.58 | 0.0002 | 0.87 | 0.1239 | 0.47 | 0.0014 |
| 2-aminobutyrate | 1.34 | 0.0004 | 1.46 | 0.0003 | 1.33 | 0.0404 |
| glycine | 0.84 | 0.0006 | 0.89 | 0.1623 | 0.86 | 0.0186 |
| N-acetylthreonine | 1.3 | 0.0006 | 1.41 | 0.0028 | 1.24 | 0.0253 |
| N-acetylisoleucine | 1.29 | 0.0011 | 1.15 | 0.2296 | 1.35 | 0.0044 |
| glycerol 3-phosphate (G3P) | 0.84 | 0.0012 | 0.68 | 0.028 | 1.02 | 0.1327 |
| allo-threonine | 0.57 | 0.0013 | 0.75 | 0.322 | 0.48 | 0.001 |
| carnitine | 1.27 | 0.0022 | 1.17 | 0.3274 | 1.39 | 0.0002 |
| theobromine | 0.79 | 0.0027 | 0.83 | 0.2223 | 0.78 | 0.0186 |
| fucose | 0.81 | 0.0032 | 0.87 | 0.0266 | 0.8 | 0.1222 |
| quinolinate | 2.04 | 0.0042 | 2.58 | 0.0024 | 1.9 | 0.3388 |
| ribitol | 1.37 | 0.0085 | 1.58 | 0.1303 | 1.45 | 0.2585 |
| azelate (nonanedioate) | 1.16 | 0.0117 | 1.17 | 0.276 | 1.17 | 0.0122 |
| threonate | 1.78 | 0.0151 | 2.92 | 0.0003 | 1.21 | 0.4008 |
| 3-carboxy-4-methyl-5-propyl-2- | 1.3 | 0.0164 | 1.62 | 9.06E−06 | 1.06 | 0.9562 |
| furanpropanoate (CMPF) | | | | | | |
| 5-methylthioadenosine (MTA) | 1.67 | 0.0177 | 0.86 | 0.0367 | 2.21 | 7.90E−06 |
| glucarate (saccharate) | 1.34 | 0.0218 | 1.44 | 0.3828 | 1.31 | 0.0478 |
| nicotinate | 1.1 | 0.0485 | 1.07 | 0.6339 | 1.14 | 0.0091 |
| 3-dehydrocarnitine | 0.98 | 0.062 | 0.93 | 0.1582 | 1.07 | 0.8919 |
| thymine | 0.79 | 0.0702 | 0.83 | 0.0277 | 0.75 | 0.5818 |
| erythronate | 0.89 | 0.0766 | 0.99 | 0.7247 | 0.89 | 0.4353 |
| 3-ureidopropionate | 1.33 | 0.0839 | 1.34 | 0.1297 | 1.36 | 0.2074 |
| N-acetylvaline | 0.97 | 0.0864 | 0.78 | 0.057 | 1.06 | 0.5605 |
| 3-hydroxybutyrate (BHBA) | 0.94 | 0.0937 | 1.04 | 0.698 | 0.89 | 0.1488 |
| gamma-glutamylleucine | 0.94 | 0.0998 | 1.33 | 0.0031 | 0.75 | 0.0003 |
| indolelactate | 0.83 | 0.1075 | 1.17 | 0.5598 | 0.72 | 0.0227 |
| pipecolate | 1.29 | 0.1524 | 1.11 | 0.7949 | 1.29 | 0.5894 |
| alpha-hydroxyisovalerate | 1.1 | 0.2137 | 1.14 | 0.1512 | 1.12 | 0.4197 |
| gamma-glutamylvaline | 0.98 | 0.2204 | 1.17 | 0.434 | 0.86 | 0.0388 |
| ascorbate (Vitamin C) | 1.12 | 0.2491 | 0.95 | 0.1257 | 1.29 | 0.418 |
| 3-methyl-2-oxovalerate | 0.9 | 0.2641 | 0.85 | 0.8026 | 0.91 | 0.3935 |
| beta-hydroxypyruvate | 1.04 | 0.3506 | 0.9 | 0.1368 | 1.1 | 0.1346 |
| N2-acetyllysine | 2.31 | 0.3516 | 2.07 | 0.6481 | 2.48 | 0.6123 |
| taurine | 1.08 | 0.3532 | 0.94 | 0.3709 | 1.22 | 0.719 |
| N-acetyltyrosine | 1.06 | 0.3873 | 0.82 | 0.0102 | 1.28 | 0.3139 |
| N-acetylglycine | 1.13 | 0.4728 | 1.01 | 0.428 | 1.2 | 0.1732 |
| 4-guanidinobutanoate | 1.2 | 0.4889 | 1.19 | 0.4321 | 1.2 | 0.7021 |
| adenine | 1.57 | 0.6044 | 0.67 | 0.0002 | 2.34 | 0.0216 |
| dimethylglycine | 1.07 | 0.711 | 0.87 | 0.656 | 1.2 | 0.1971 |
| cysteine | 1.46 | 0.7909 | 1.27 | 0.271 | 1.69 | 0.2777 |
| xylonate | 0.9 | 0.7933 | 1.15 | 0.129 | 0.83 | 0.6313 |

The biomarkers were used to create a statistical model to classify the samples. Using Random Forest analysis, the biomarkers were used in a mathematical model to classify samples as Normal tissue or as Tumor (cancer). Samples from patient-matched kidney tumor and normal tissue from 140 subjects were used in this analysis.

Random Forest results show that the samples were classified with 99% prediction accuracy. The Confusion Matrix presented in Table 5 shows the number of samples predicted for each classification and the actual in each group (Tumor or Normal). The “Out-of-Bag” (OOB) Error rate gives an estimate of how accurately new observations can be predicted using the Random Forest model (e.g., whether a sample is from tumor tissue or normal tissue). The OOB error from this Random Forest was approximately 1%, and the model estimated that, when used on a new set of subjects, the identity of normal subjects could be predicted correctly 98% of the time and kidney cancer subjects could be predicted 100% of the time.

TABLE 5
Results of Random Forest: Kidney Tumor vs. Normal

| | | Predicted Group | | Class |
|---|---|---|---|---|
| | | Normal | Tumor | Error |
| Actual | Normal | 137 | 3 | 0.0214 |
| Group | Tumor | 1 | 139 | 0.0071 |

Predictive accuracy = 99%

Based on the OOB Error rate of 1%, the Random Forest model that was created predicted the tumor status of a sample with about 99% accuracy based on the levels of the biomarkers measured in samples from the subject. Exemplary biomarkers for distinguishing the groups are N-acetylaspartate (NAA), maltose, N-acetyl-aspartyl-glutamate (NAAG), 1-palmitoylglycerophosphoethanolamine, phenylacetylglutamine, glucose 6-phosphate (G6P), 1-oleoylglycerophosphoethanolamine, pseudouridine, maltotriose, N6-acetyllysine, 2-oleoylglycerophosphoethanolamine, glucose, eicosenoate (20:1n9 or 1n11), fructose-6-phosphate, 1-palmitoylglycerophosphoinositol, maltotetraose, N1-methylguanosine, 2-palmitoylglycerophosphoethanolamine, dimethylarginine (ADMA+SDMA), N1-methyladenosine, pantothenate, malonylcarnitine, arachidonate (20:4n6), 1-palmitoylplasmenylethanolamine, hippurate, 1-stearoylglycerophosphoethanolamine, kynurenine, alpha-tocopherol, fructose 1-phosphate, and 1-stearoylglycerophosphoinositol.

The Random Forest analysis demonstrated that by using the biomarkers, tumor samples were distinguished from Normal samples with 99% sensitivity, 98% specificity, 98% PPV and 99% NPV.

The biomarkers were used to create a statistical model to classify the early stage (T1) samples. Using Random Forest analysis, the biomarkers were used in a mathematical model to classify samples as Normal or as tumor. Samples from patient-matched kidney tumor and normal tissue from 43 subjects with Stage 1 (T1) kidney cancer were used in this analysis.

Random Forest results show that the samples were classified with 99% prediction accuracy. The Confusion Matrix presented in Table 6 shows the number of samples predicted for each classification and the actual in each group (T1 Tumor or T1 Normal). The “Out-of-Bag” (OOB) Error rate gives an estimate of how accurately new observations can be predicted using the Random Forest model (e.g., whether a sample is from tumor tissue or normal tissue). The OOB error from this Random Forest was approximately 1%, and the model estimated that, when used on a new set of subjects, the identity of normal subjects could be predicted correctly 98% of the time and kidney cancer subjects could be predicted 100% of the time.

TABLE 6
Results of Random Forest: Kidney T1 Tumor vs. T1 Normal

| | | Predicted Group | | Class |
|---|---|---|---|---|
| | | Normal | Tumor | Error |
| Actual | Normal | 42 | 1 | 0.0233 |
| Group | Tumor | 0 | 43 | 0 |

Predictive accuracy = 99%

Based on the OOB Error rate of 1%, the Random Forest model that was created predicted the tumor status of a sample with about 99% accuracy based on the levels of the biomarkers measured in samples from the subjects. Exemplary biomarkers for distinguishing the groups are N-acetylaspartate (NAA), 1-oleoyl-GPE (18:1), N-acetyl-aspartyl-glutamate (NAAG), 1-palmitoyl-GPE (16:0), maltose, 2-oleoyl-GPE (18:1), eicosenoate (20:1n9 or 1n11), 1-palmitoyl-GPI (16:0), 2-palmitoyl-GPE (16:0), 1-stearoyl-GPI (18:0), N2-methylguanosine, phenylacetylglutamine, N-acetylneuraminate, beta-alanine, malonylcarnitine, fructose 6-phosphate, gamma-glutamylglutamate, FAD, pseudouridine, 1-methylguanisine, 1-stearoyl-GPE (18:0), citrate, pantothenate (Vitamin B5), 1-palmitoylplasmenylethanolamine, arachidonate (20:4n6), N6-acetyllysine, 1-oleoyl-GPI (18:1), 2-methylbutyroylcarnitine (C5), fructose 1-phosphate, alpha-tocopherol.

The Random Forest analysis demonstrated that by using the biomarkers, tumor samples were distinguished from Normal samples with 100% sensitivity, 98% specificity, 98% PPV and 100% NPV.

The biomarkers were used to create a statistical model to classify the samples. Using Random Forest analysis, the biomarkers were used in a mathematical model to classify samples as Normal or as tumor. Samples from patient-matched kidney tumor and normal tissue from 80 subjects with Stage 3 (T3) kidney cancer were used in this analysis.

Random Forest results show that the samples were classified with 98% prediction accuracy. The Confusion Matrix presented in Table 7 shows the number of samples predicted for each classification and the actual in each group (T3 Tumor or T3 Normal). The “Out-of-Bag” (OOB) Error rate gives an estimate of how accurately new observations can be predicted using the Random Forest model (e.g., whether a sample is from tumor tissue or normal tissue). The OOB error from this Random Forest was approximately 2%, and the model estimated that, when used on a new set of subjects, the identity of normal subjects could be predicted correctly 96% of the time and kidney cancer subjects could be predicted 99% of the time.

TABLE 7
Results of Random Forest: Kidney T3 Tumor vs. T3 Normal

| | | Predicted Group | | Class |
|---|---|---|---|---|
| | | Normal | Tumor | Error |
| Actual | Normal | 77 | 3 | 0.0375 |
| Group | Tumor | 1 | 79 | 0.0125 |

Predictive accuracy = 98%

Based on the OOB Error rate of 2%, the Random Forest model that was created predicted the tumor status of a sample with about 98% accuracy based on the levels of the biomarkers measured in samples from the subject. Exemplary biomarkers for distinguishing the groups are maltose, N-acetylaspartate (NAA), N-acetyl-aspartyl-glutamate (NAAG), glucose 6-phosphate (G6P), maltotetraose, phenylacetylglutamine, maltotriose, pseudouridine, 1-palmitoylglycerophosphoethanolamine, N1-methylguanosine, methyl-alpha-glucopyranoside, fructose-6-phosphate, 1-oleoylglycerophosphoethanolamine, N6-acetyllysine, dimethylarginine (ADMA+SDMA), 1-palmitoylglycerophosphoinositol, hippurate, N1-methyladenosine, mannose-6-phosphate, eicosenoate (20:1n9 or 11), glucose, pantothenate, 2-oleoylglycerophosphoethanolamine, alpha-tocopherol, 2-hydroxyglutarate, 2-palmitoylglycerophosphoethanolamine, arabitol, malonylcarnitine, arachidonate (20:4n6), and ergothioneine.

The Random Forest analysis demonstrated that by using the biomarkers, tumor samples were distinguished from Normal samples with 99% sensitivity, 96% specificity, 96% PPV and 99% NPV.

### Example 4

### Tissue Biomarkers for Staging Kidney Cancer

Kidney cancer staging provides an indication of how far the kidney tumor has spread beyond the kidney. The tumor stage is used to select treatment options and to estimate a patient's prognosis. Kidney tumor stages range from T1 (tumor 7 cm or less in size and limited to kidney, least advanced) to T4 (tumor invades beyond Gerota's fascia, most advanced).

To identify biomarkers of kidney cancer stage, metabolomic analysis was carried out on tissue samples from 56 subjects with Low stage RCC (T1, T2) and 84 subjects with High stage RCC (T3,T4). After the levels of metabolites were determined, the data were analyzed using Welch's two-sample t-test to identify biomarkers that differed between low stage kidney cancer compared to high stage kidney cancer. The biomarkers are listed in Table 8.

Table 8 includes, for each biomarker, the biochemical name of the biomarker, the fold change (FC) of the biomarker in high stage kidney cancer compared to low stage kidney cancer (T3,T4 Tumor/T1,T2 Tumor) and the p-value determined in the statistical analysis of the data concerning the biomarkers. Columns 4 and 5 of Table 8 include the identifier for that biomarker compound in the Kyoto Encyclopedia of Genes and Genomes (KEGG), if available; and the identifier for that biomarker compound in the Human Metabolome Database (HMDB), if available. Bold values indicate a fold of change with a p-value of <0.1.

TABLE 8
Tissue Biomarkers for Kidney Cancer Staging

| | T3-T4-TUMOR | | | |
|---|---|---|---|---|
| | T1-T2-TUMOR | | | |
| Biochemical Name | FC | p-value | KEGG | HMDB |
| laurate (12:0) | 0.66 | 1.78E−07 | C02679 | HMDB00638 |
| pelargonate (9:0) | 0.72 | 1.16E−06 | C01601 | HMDB00847 |
| homocysteine | 2.45 | 7.32E−06 | C00155 | HMDB00742 |
| arginine | 1.35 | 4.62E−05 | C00062 | HMDB00517 |
| ribose | 1.76 | 5.02E−05 | C00121 | HMDB00283 |
| 2-ethylhexanoate | 0.56 | 9.99E−05 | | |
| inositol 1-phosphate (I1P) | 0.61 | 0.0004 | | HMDB00213 |
| guanosine 5′-monophosphate (5′-GMP) | 0.59 | 0.0073 | | |
| 4-hydroxybutyrate (GHB) | 2.59 | 6.60E−06 | C00989 | HMDB00710 |
| lysylmethionine | 2.27 | 9.77E−08 | | |
| glutathione, reduced (GSH) | 10.33 | 4.58E−06 | C00051 | HMDB00125 |
| cytidine 5′-diphosphocholine | 2.03 | 3.74E−05 | | |
| glycylisoleucine | 1.75 | 4.20E−05 | | |
| isoleucyltryptophan | 2.98 | 6.36E−05 | | |
| aspartylphenylalanine | 1.78 | 6.91E−05 | | HMDB00706 |
| S-adenosylmethionine (SAM) | 1.55 | 9.03E−05 | | |
| valerylcarnitine | 1.69 | 9.85E−05 | | HMDB13128 |
| galactose | 1.93 | 0.0001 | C01582 | HMDB00143 |
| glucose 1-phosphate | 0.51 | 0.0001 | C00103 | HMDB01586 |
| alanylglycine | 1.82 | 0.0001 | | HMDB06899 |
| alanylisoleucine | 2.18 | 0.0001 | | |
| isoleucylmethionine | 2.66 | 0.0001 | | |
| aspartylleucine | 1.79 | 0.0001 | | |
| methionylalanine | 2.79 | 0.0001 | | |
| glycylthreonine | 1.72 | 0.0001 | | |
| asparagine | 1.6 | 0.0002 | C00152 | HMDB00168 |
| isoleucylglycine | 1.62 | 0.0002 | | |
| caprate (10:0) | 0.81 | 0.0003 | C01571 | HMDB00511 |
| tryptophylasparagine | 2.1 | 0.0003 | | |
| 2′-deoxyinosine | 1.93 | 0.0004 | C05512 | HMDB00071 |
| homoserine | 1.87 | 0.0004 | C00263 | HMDB00719 |
| nicotinamide | 1.3 | 0.0005 | C00153 | HMDB01406 |
| alanylglutamate | 1.83 | 0.0005 | | |
| tyrosylalanine | 1.68 | 0.0005 | | |
| serylisoleucine | 1.62 | 0.0005 | | |
| cytosine-2′,3′-cyclic monophosphate | 1.72 | 0.0006 | C02354 | HMDB11691 |
| isoleucylhistidine | 1.46 | 0.0006 | | |
| aspartyltryptophan | 1.63 | 0.0006 | | |
| valylglycine | 1.81 | 0.0007 | | |
| xylitol | 1.61 | 0.0007 | C00379 | HMDB00568 |
| prolylmethionine | 1.77 | 0.0007 | | |
| myristate (14:0) | 0.84 | 0.0009 | C06424 | HMDB00806 |
| butyrylcarnitine | 1.39 | 0.0009 | | |
| aspartate-glutamate | 1.66 | 0.0009 | | |
| phenylalanylserine | 1.87 | 0.0009 | | |
| isoleucylvaline | 2.04 | 0.0009 | | |
| tyrosylglycine | 1.38 | 0.0009 | | |
| histidyltryptophan | 1.94 | 0.0009 | | |
| lysyltyrosine | 3.27 | 0.0009 | | |
| glycyltryptophan | 1.82 | 0.001 | | |
| threonylmethionine | 1.91 | 0.0012 | | |
| glycylvaline | 1.47 | 0.0013 | | |
| leucyltryptophan | 1.53 | 0.0013 | | |
| isoleucylalanine | 2.01 | 0.0014 | | |
| valylglutamate | 1.6 | 0.0015 | | |
| leucylserine | 2.01 | 0.0023 | | |
| methionylglycine | 2.14 | 0.0024 | | |
| aspartylvaline | 3.04 | 0.0024 | | |
| caprylate (8:0) | 0.77 | 0.0028 | C06423 | HMDB00482 |
| methionylleucine | 2.13 | 0.0028 | | |
| leucylphenylalanine | 1.79 | 0.0029 | | |
| isoleucylglutamate | 1.79 | 0.0029 | | |
| isoleucylphenylalanine | 2.28 | 0.0031 | | |
| valylphenylalanine | 2.26 | 0.0031 | | |
| 3-hydroxyhippurate | 2.45 | 0.0032 | | HMDB06116 |
| phenylalanylalanine | 1.77 | 0.0036 | | |
| valylvaline | 1.98 | 0.0037 | | |
| alanylvaline | 1.7 | 0.0038 | | |
| 2-eicosatrienoylglycerophosphocholine | 2.04 | 0.0039 | | |
| phenylalanylaspartate | 1.64 | 0.0039 | | |
| 2′-deoxyguanosine | 1.66 | 0.0044 | C00330 | HMDB00085 |
| tyrosylvaline | 1.61 | 0.0044 | | |
| mannose-6-phosphate | 1.33 | 0.0045 | C00275 | HMDB01078 |
| methionylasparagine | 1.63 | 0.0046 | | |
| tryptophylglutamate | 1.42 | 0.0047 | | |
| glycylleucine | 1.39 | 0.0048 | C02155 | HMDB00759 |
| alanylphenylalanine | 2.21 | 0.0048 | | |
| caproate (6:0) | 0.83 | 0.0053 | C01585 | HMDB00535 |
| lysylleucine | 1.7 | 0.0054 | | |
| valyltyrosine | 1.9 | 0.0059 | | |
| 2-arachidonoylglycerophosphoethanolamine | 1.28 | 0.0068 | | |
| serylleucine | 1.92 | 0.0068 | | |
| valylalanine | 1.83 | 0.0068 | | |
| histidyltyrosine | 1.46 | 0.0073 | | |
| agmatine | 2.06 | 0.0074 | C00179 | HMDB01432 |
| phenylalanylglutamate | 2.13 | 0.0076 | | |
| alanylleucine | 2.25 | 0.0077 | | |
| N-acetylmethionine | 1.4 | 0.0079 | C02712 | HMDB11745 |
| citrulline | 0.8 | 0.0079 | C00327 | HMDB00904 |
| valylaspartate | 1.72 | 0.0079 | | |
| valylasparagine | 2.13 | 0.0079 | C00252 | HMDB02923 |
| tyrosylleucine | 1.79 | 0.0086 | | |
| cysteinylglycine | 4.01 | 0.0089 | C01419 | HMDB00078 |
| valylmethionine | 2.26 | 0.009 | | |
| phenylalanylglycine | 1.94 | 0.0092 | | |
| spermidine | 1.26 | 0.0097 | C00315 | HMDB01257 |
| phenylalanylvaline | 1.74 | 0.0099 | | |
| threonylphenylalanine | 1.73 | 0.01 | | |
| leucyltyrosine | 1.57 | 0.0102 | | |
| N-acetylglucosamine 6-phosphate | 1.35 | 0.0103 | C00357 | HMDB02817 |
| phenylalanyltyrosine | 1.54 | 0.0116 | | |
| histidylleucine | 1.46 | 0.0117 | | |
| glycylmethionine | 1.56 | 0.0118 | | |
| leucylmethionine | 1.81 | 0.0127 | | |
| valylhistidine | 1.92 | 0.0128 | | |
| 3′-dephosphocoenzyme A | 1.41 | 0.013 | C00882 | HMDB01373 |
| leucylglycine | 2.19 | 0.013 | | |
| 2-palmitoleoylglycerophosphocholine | 1.42 | 0.0131 | | |
| isoleucylarginine | 1.31 | 0.0131 | | |
| gamma-glutamylcysteine | 1.32 | 0.0132 | C00669 | HMDB01049 |
| valylisoleucine | 1.91 | 0.0133 | | |
| valyllysine | 1.9 | 0.0142 | | |
| serylvaline | 1.49 | 0.0144 | | |
| isoleucyltyrosine | 1.81 | 0.0147 | | |
| threonylglutamate | 1.64 | 0.0151 | | |
| uridine monophosphate (5′ or 3′) | 0.7 | 0.0154 | | |
| glycyltyrosine | 1.31 | 0.0155 | | |
| dihydrocholesterol | 1.17 | 0.0157 | | HMDB00908 |
| 3-(4-hydroxyphenyl)lactate | 1.42 | 0.0164 | C03672 | HMDB00755 |
| histidylmethionine | 1.65 | 0.0169 | | |
| phosphate | 1.22 | 0.0175 | C00009 | HMDB01429 |
| alpha-glutamyltyrosine | 1.55 | 0.0175 | | |
| histidylphenylalanine | 1.55 | 0.0182 | | |
| leucylglutamate | 1.86 | 0.0183 | | |
| valylglutamine | 1.69 | 0.0191 | | |
| glycylphenylalanine | 1.52 | 0.0202 | | |
| 1,3-dihydroxyacetone | 1.39 | 0.0203 | C00184 | HMDB01882 |
| alanylthreonine | 1.48 | 0.0203 | | |
| leucylarginine | 1.51 | 0.021 | | |
| putrescine | 1.17 | 0.0211 | C00134 | HMDB01414 |
| cytidine | 1.35 | 0.0214 | C00475 | HMDB00089 |
| trans-4-hydroxyproline | 2.46 | 0.0214 | C01157 | HMDB00725 |
| tyrosylglutamine | 1.44 | 0.0215 | | |
| glucose-6-phosphate (G6P) | 1.29 | 0.0217 | C00668 | HMDB01401 |
| 2-oleoylglycerophosphoserine | 1.13 | 0.0248 | | |
| alpha-glutamyltryptophan | 1.68 | 0.0248 | | |
| testosterone | 0.8 | 0.0249 | C00535 | HMDB00234 |
| 1-heptadecanoylglycerophosphoethanolamine | 1.93 | 0.0252 | | |
| leucylalanine | 1.81 | 0.0252 | | |
| VGAHAGEYGAEALER | 0.92 | 0.0253 | | |
| adenosine 2′-monophosphate (2′-AMP) | 1.22 | 0.0257 | C00946 | HMDB11617 |
| valylserine | 1.98 | 0.0261 | | |
| cystine | 0.86 | 0.0264 | C00491 | HMDB00192 |
| arginylleucine | 1.76 | 0.0264 | | |
| bilirubin (E,E) | 0.7 | 0.0268 | | |
| myristoleate (14:1n5) | 0.89 | 0.0275 | C08322 | HMDB02000 |
| threonylleucine | 1.71 | 0.0285 | | |
| phenylalanylarginine | 1.97 | 0.0291 | | |
| guanine | 0.54 | 0.0294 | C00242 | HMDB00132 |
| isoleucylserine | 1.8 | 0.0299 | | |
| Isobar: fructose 1,6-diphosphate, glucose 1,6- | 0.73 | 0.0314 | | |
| diphosphate, myo-inositol 1,4 or | | | | |
| 1,3-diphosphate | | | | |
| leucylleucine | 1.62 | 0.032 | C11332 | |
| phenylalanylproline | 1.55 | 0.0323 | | |
| 2-linoleoylglycerophosphocholine | 1.4 | 0.0333 | | |
| 16-hydroxypalmitate | 0.86 | 0.0336 | C18218 | |
| lysyllysine | 1.31 | 0.0347 | | |
| N-acetylalanine | 1.19 | 0.0365 | C02847 | HMDB00766 |
| phenylalanyltryptophan | 1.36 | 0.0376 | | |
| 7-alpha-hydroxy-3-oxo-4-cholestenoate | 1.65 | 0.038 | C17337 | HMDB12458 |
| (7-Hoca) | | | | |
| arginylvaline | 1.25 | 0.038 | | |
| alanylmethionine | 1.89 | 0.0387 | | |
| valyltryptophan | 1.7 | 0.0388 | | |
| 6′-sialyllactose | 1.49 | 0.039 | G00265 | HMDB06569 |
| threonylvaline | 1.66 | 0.0406 | | |
| serylphenyalanine | 1.55 | 0.0408 | | |
| 2-arachidonoylglycerophosphocholine | 1.56 | 0.0411 | | |
| bilirubin (Z,Z) | 0.59 | 0.0419 | C00486 | HMDB00054 |
| ribulose | 1.32 | 0.042 | C00309 | HMDB00621 |
| | | | | HMDB03371 |
| alanylalanine | 1.27 | 0.0423 | C00993 | HMDB03459 |
| heme | 0.64 | 0.0424 | | |
| valylleucine | 2.26 | 0.0428 | | |
| 2′-deoxyadenosine 3′-monophosphate | 1.36 | 0.0436 | | |
| 2-palmitoylglycerol (2-monopalmitin) | 1.21 | 0.0462 | | |
| dihomo-linolenate (20:3n3 or n6) | 1.27 | 0.0462 | C03242 | HMDB02925 |
| ophthalmate | 1.42 | 0.0464 | | HMDB05765 |
| 3-hydroxyoctanoate | 1.18 | 0.049 | | HMDB01954 |
| leucylasparagine | 1.59 | 0.0517 | | |
| arginylmethionine | 1.44 | 0.0519 | | |
| 2-docosapentaenoylglycerophosphocholine | 1.44 | 0.0532 | | |
| deoxycarnitine | 1.15 | 0.0544 | C01181 | HMDB01161 |
| docosatrienoate (22:3n3) | 1.34 | 0.0566 | C16534 | HMDB02823 |
| 2-hydroxypalmitate | 1.67 | 0.0595 | | |
| sedoheptulose-7-phosphate | 1.25 | 0.0636 | C05382 | HMDB01068 |
| 1,2-propanediol | 1.22 | 0.0637 | C00583 | HMDB01881 |
| glutathione, oxidized (GSSG) | 2.04 | 0.0688 | C00127 | HMDB03337 |
| urea | 1.26 | 0.0728 | C00086 | HMDB00294 |
| alanyltyrosine | 1.45 | 0.074 | | |
| glycylglycine | 1.44 | 0.0789 | C02037 | HMDB11733 |
| N-acetylserine | 1.27 | 0.0838 | | HMDB02931 |
| arginyltyrosine | 1.4 | 0.0923 | | |
| maltohexaose | 0.75 | 0.0928 | C01936 | HMDB12253 |
| phenylalanylleucine | 1.66 | 0.0928 | | |
| arabonate | 1.31 | 0.0929 | | HMDB00539 |
| thymidine | 1.16 | 0.0931 | C00214 | HMDB00273 |
| alpha-glutamylglutamate | 1.61 | 0.0934 | C01425 | |
| gamma-glutamylglutamate | 0.76 | 0.0951 | | |
| tyrosyllysine | 2.17 | 0.0973 | | |
| 2-docosapentaenoylglycerophosphoethanolamine | 0.78 | 0.1003 | | |
| 2-linoleoylglycerophosphoethanolamine | 1.2 | 0.1008 | | |
| N-acetylornithine | 0.94 | 0.1037 | C00437 | HMDB03357 |
| 6-phosphogluconate | 1.46 | 0.1065 | C00345 | HMDB01316 |
| fructose-6-phosphate | 1.17 | 0.1075 | C05345 | HMDB00124 |
| tyrosyltyrosine | 1.39 | 0.1082 | | |
| phosphoethanolamine | 1.14 | 0.1088 | C00346 | HMDB00224 |
| arginylphenylalanine | 1.5 | 0.1107 | | |
| 2-oleoylglycerophosphocholine | 1.51 | 0.1137 | | |
| maltotetraose | 0.69 | 0.1147 | C02052 | HMDB01296 |
| 4-hydroxyglutamate | 1.66 | 0.1166 | C03079 | HMDB01344 |
| N-acetyltryptophan | 2.91 | 0.1178 | C03137 | |
| spermine | 2.08 | 0.1336 | C00750 | HMDB01256 |
| dodecanedioate | 0.83 | 0.1358 | C02678 | HMDB00623 |
| 2-stearoylglycerophosphoethanolamine | 1.13 | 0.1375 | | |
| gamma-tocopherol | 0.8 | 0.1403 | C02483 | HMDB01492 |
| phenylalanylphenylalanine | 1.49 | 0.1446 | | |
| methionylglutamate | 1.39 | 0.1564 | | |
| choline phosphate | 0.9 | 0.1585 | | |
| 2-oleoylglycerol (2-monoolein) | 1.24 | 0.164 | | |
| tyrosylhistidine | 1.38 | 0.1653 | | |
| 7-alpha-hydroxycholesterol | 1.75 | 0.167 | C03594 | HMDB01496 |
| methionylaspartate | 1.56 | 0.1679 | | |
| 1-palmitoleoylglycerophosphocholine | 1.33 | 0.1718 | | |
| adrenate (22:4n6) | 1.12 | 0.1861 | C16527 | HMDB02226 |
| pyridoxal | 1.14 | 0.1869 | C00250 | HMDB01545 |
| 1-stearoylglycerophosphoinositol | 1.28 | 0.1869 | | |
| 1-oleoylglycerophosphocholine | 1.4 | 0.1898 | | |
| beta-tocopherol | 0.79 | 0.1941 | C14152 | HMDB06335 |
| tryptophylleucine | 1.38 | 0.2027 | | |
| isoleucylisoleucine | 1.51 | 0.2093 | | |
| 1-palmitoylglycerophosphoinositol | 1.14 | 0.2119 | | |
| uridine | 1.1 | 0.2138 | C00299 | HMDB00296 |
| 15-methylpalmitate (isobar with 2- | 0.93 | 0.2288 | | |
| methylpalmitate) | | | | |
| tyrosylphenylalanine | 1.12 | 0.2336 | | |
| N-methylglutamate | 1.81 | 0.2357 | C01046 | |
| leucylhistidine | 1.37 | 0.2423 | | |
| cytidine-3′-monophosphate (3′-CMP) | 1.19 | 0.2435 | C05822 | |
| maltotriose | 0.85 | 0.2474 | C01835 | HMDB01262 |
| 1-arachidonoylglycerophosphocholine | 1.3 | 0.2594 | C05208 | |
| linolenate [alpha or gamma; (18:3n3 or 6)] | 0.91 | 0.2599 | C06427 | HMDB01388 |
| 2-docosahexaenoylglycerophosphoethanolamine | 0.8 | 0.2601 | | |
| nicotinamide ribonucleotide (NMN) | 0.86 | 0.265 | C00455 | HMDB00229 |
| dihomo-linoleate (20:2n6) | 1.07 | 0.2651 | C16525 | |
| stearate (18:0) | 0.94 | 0.269 | C01530 | HMDB00827 |
| linoleate (18:2n6) | 0.92 | 0.2714 | C01595 | HMDB00673 |
| pyrophosphate (PPi) | 0.86 | 0.2716 | C00013 | HMDB00250 |
| 1-stearoylglycerol (1-monostearin) | 0.89 | 0.273 | D01947 | |
| flavin adenine dinucleotide (FAD) | 1.1 | 0.2752 | C00016 | HMDB01248 |
| 13-HODE +9-HODE | 0.73 | 0.2837 | | |
| adenosine 3′-monophosphate (3′-AMP) | 1.21 | 0.284 | C01367 | HMDB03540 |
| 3-phosphoglycerate | 0.97 | 0.2876 | C00597 | HMDB00807 |
| erucate (22:1n9) | 0.86 | 0.293 | C08316 | HMDB02068 |
| cytidine 5′-monophosphate (5′-CMP) | 1.14 | 0.2937 | C00055 | HMDB00095 |
| S-methylcysteine | 1.13 | 0.3022 | | HMDB02108 |
| glycerate | 1.17 | 0.3074 | C00258 | HMDB00139 |
| oleoylcarnitine | 1.04 | 0.3201 | | HMDB05065 |
| 5-methyluridine (ribothymidine) | 1.01 | 0.3202 | | HMDB00884 |
| 1-myristoylglycerophosphoethanolamine | 1 | 0.3202 | | HMDB11500 |
| methionylphenylalanine | 0.97 | 0.3209 | | |
| adenosine 5′-monophosphate (AMP) | 0.85 | 0.3289 | C00020 | HMDB00045 |
| 2-oleoylglycerophosphoethanolamine | 1.19 | 0.335 | | |
| glycerol 2-phosphate | 1.17 | 0.3378 | C02979 | HMDB02520 |
| 2′-deoxycytidine 3′-monophosphate | 1.32 | 0.3429 | | |
| ethanolamine | 1.12 | 0.3446 | C00189 | HMDB00149 |
| undecanedioate | 1.05 | 0.3449 | | HMDB00888 |
| phenylalanylmethionine | 1.41 | 0.3499 | | |
| prolylglycine | 1.22 | 0.3521 | | |
| methyl-alpha-glucopyranoside | 0.92 | 0.359 | C02603 | |
| 1-myristoylglycerophosphocho line | 1.27 | 0.3722 | | HMDB10379 |
| ergothioneine | 1.11 | 0.3762 | C05570 | HMDB03045 |
| arachidate (20:0) | 0.95 | 0.3782 | C06425 | HMDB02212 |
| 2-palmitoylglycerophosphocholine | 1.28 | 0.3785 | | |
| 2-linoleoylglycerol (2-monolinolein) | 0.91 | 0.3788 | | HMDB11538 |
| palmitate (16:0) | 0.95 | 0.3812 | C00249 | HMDB00220 |
| methylphosphate | 0.97 | 0.3818 | | |
| margarate (17:0) | 0.94 | 0.3828 | | HMDB02259 |
| alanyltryptophan | 0.99 | 0.3891 | | |
| Ac-Ser-Asp-Lys-Pro-OH | 1.02 | 0.3919 | | |
| glycyllysine | 1.43 | 0.3928 | | |
| valylarginine | 1.02 | 0.4048 | | |
| 3,4-dihydroxyphenethyleneglycol | 1.07 | 0.4052 | C05576 | HMDB00318 |
| 5-oxoETE | 0.88 | 0.4116 | C14732 | HMDB10217 |
| docosapentaenoate (n6 DPA; 22:5n6) | 1.16 | 0.4121 | C06429 | HMDB13123 |
| 5-HETE | 0.8 | 0.4208 | | |
| stearoylcarnitine | 1.33 | 0.4226 | | HMDB00848 |
| cholesterol | 1.08 | 0.4227 | C00187 | HMDB00067 |
| 1-pentadecanoylglycerophosphocholine | 1.28 | 0.4281 | | |
| glycerophosphoethanolamine | 1.41 | 0.4285 | C01233 | HMDB00114 |
| 1-oleoylglycerophosphoethanolamine | 1.27 | 0.4334 | | HMDB11506 |
| 1-linoleoylglycerophosphocholine | 1.15 | 0.4349 | C04100 | |
| 1-palmitoylplasmenylethanolamine | 1.06 | 0.4451 | | |
| imidazole propionate | 1.48 | 0.4462 | | HMDB02271 |
| maltopentaose | 0.77 | 0.4504 | C06218 | HMDB12254 |
| triethyleneglycol | 1.09 | 0.4541 | | |
| 1-palmitoylglycerophosphocholine | 1.03 | 0.4648 | | |
| Isobar: ribulose 5-phosphate, xylulose | 1.08 | 0.4651 | | |
| 5-phosphate | | | | |
| 1-stearoylglycerophosphoethanolamine | 1.09 | 0.4718 | | HMDB11130 |
| inosine | 1.04 | 0.4725 | | |
| nicotinamide adenine dinucleotide reduced | 0.88 | 0.4747 | C00004 | HMDB01487 |
| (NADH) | | | | |
| sphinganine | 1.17 | 0.4777 | C00836 | HMDB00269 |
| phytosphingosine | 1.15 | 0.4789 | C12144 | HMDB04610 |
| cysteine-glutathione disulfide | 1.61 | 0.4798 | | HMDB00656 |
| alpha-tocopherol | 0.92 | 0.4869 | C02477 | HMDB01893 |
| cis-vaccenate (18:1n7) | 0.98 | 0.4893 | C08367 | |
| arabitol | 1.17 | 0.4953 | C00474 | HMDB01851 |
| palmitoleate (16:1n7) | 0.93 | 0.5007 | C08362 | HMDB03229 |
| 1-arachidonoylglycerophosphoinositol | 0.99 | 0.5024 | | |
| betaine | 0.93 | 0.5137 | | HMDB00043 |
| palmitoylcarnitine | 1.08 | 0.5141 | | |
| 7-beta-hydroxycholesterol | 1.3 | 0.5168 | C03594 | HMDB06119 |
| stearidonate (18:4n3) | 0.95 | 0.5205 | C16300 | HMDB06547 |
| argininosuccinate | 1.31 | 0.5259 | C03406 | HMDB00052 |
| 1-arachidonoylglycerophosphoethanolamine | 1.02 | 0.5265 | | HMDB11517 |
| docosadienoate (22:2n6) | 0.99 | 0.5352 | C16533 | |
| ornithine | 1.32 | 0.5601 | C00077 | HMDB03374 |
| glutamate, gamma-methyl ester | 1.12 | 0.5676 | | |
| cinnamoylglycine | 0.99 | 0.5701 | | |
| adenylosuccinate | 0.87 | 0.5734 | C03794 | HMDB00536 |
| 2-myristoylglycerophosphocholine | 1 | 0.5844 | | |
| arachidonate (20:4n6) | 0.98 | 0.5993 | C00219 | HMDB01043 |
| 2-palmitoylglycerophosphoethanolamine | 1.24 | 0.6045 | | |
| 1-stearoylglycerophosphocholine | 1.15 | 0.6215 | | |
| 1-palmitoleoylglycerophosphoethanolamine | 0.97 | 0.6247 | | |
| 5-methyltetrahydrofolate (5MeTHF) | 0.99 | 0.6345 | C00440 | HMDB01396 |
| 2-phosphoglycerate | 1.04 | 0.6516 | C00631 | HMDB03391 |
| gamma-glutamylglutamine | 1.53 | 0.6572 | | HMDB11738 |
| N1-Methyl-2-pyridone-5-carboxamide | 1.04 | 0.6632 | C05842 | HMDB04193 |
| saccharopine | 1.34 | 0.664 | C00449 | HMDB00279 |
| 1-arachidonylglycerol | 0.96 | 0.6669 | C13857 | HMDB11572 |
| phosphoenolpyruvate (PEP) | 1.1 | 0.6688 | C00074 | HMDB00263 |
| 6-keto prostaglandin Flalpha | 1.25 | 0.6797 | C05961 | HMDB02886 |
| 1-docosahexaenoylglycerophosphocholine | 1.07 | 0.6855 | | |
| nicotinamide adenine dinucleotide (NAD+) | 1.29 | 0.6861 | C00003 | HMDB00902 |
| maltose | 1.06 | 0.691 | C00208 | HMDB00163 |
| pentadecanoate (15:0) | 1 | 0.6963 | C16537 | HMDB00826 |
| oleate (18:1n9) | 0.9 | 0.7 | C00712 | HMDB00207 |
| 2-docosahexaenoylglycerophosphocholine | 1.08 | 0.7031 | | |
| palmitoyl sphingomyelin | 0.97 | 0.7068 | | |
| eicosenoate (20:1n9 or 11) | 0.91 | 0.7232 | | HMDB02231 |
| piperine | 0.95 | 0.7288 | C03882 | |
| nervonate (24:1n9) | 0.98 | 0.7451 | C08323 | HMDB02368 |
| hypotaurine | 1.01 | 0.7604 | C00519 | HMDB00965 |
| 1-palmitoylglycerophosphoethanolamine | 1.19 | 0.7781 | | HMDB11503 |
| sphingosine | 1.28 | 0.7939 | C00319 | HMDB00252 |
| 1-oleoylglycerol (1-monoolein) | 1.03 | 0.7969 | | HMDB11567 |
| prostaglandin A2 | 1.07 | 0.7971 | C05953 | HMDB02752 |
| 1-oleoylglycerophosphoserine | 1.03 | 0.8021 | | |
| fructose 1-phosphate | 0.83 | 0.8127 | C01094 | HMDB01076 |
| 1-linoleoylglycerophosphoethanolamine | 0.99 | 0.8379 | | HMDB11507 |
| prostaglandin E2 | 1.43 | 0.8423 | C00584 | HMDB01220 |
| 1-palmitoylglycerol (1-monopalmitin) | 0.94 | 0.8438 | | |
| N-acetylglucosamine | 1.36 | 0.8453 | C00140 | HMDB00215 |
| sorbitol 6-phosphate | 0.92 | 0.8477 | C01096 | HMDB05831 |
| 1-heptadecanoylglycerophosphocholine | 1.12 | 0.8515 | | HMDB12108 |
| pregnanediol-3-glucuronide | 1 | 0.856 | | |
| guanosine | 1 | 0.8626 | C00387 | HMDB00133 |
| 3-hydroxydecanoate | 1.02 | 0.863 | | HMDB02203 |
| 10-heptadecenoate (17:1n7) | 0.98 | 0.8818 | | |
| laurylcarnitine | 1.07 | 0.8844 | | HMDB02250 |
| myristoylcarnitine | 1.06 | 0.8978 | | |
| squalene | 0.88 | 0.9086 | C00751 | HMDB00256 |
| cortisol | 0.92 | 0.9148 | C00735 | HMDB00063 |
| 1-oleoylglycerophosphoinositol | 1.02 | 0.9196 | | |
| docosapentaenoate (n3 DPA; 22:5n3) | 0.93 | 0.922 | C16513 | HMDB01976 |
| 2-stearoylglycerophosphocholine | 1.13 | 0.9348 | | |
| histamine | 1.08 | 0.9451 | C00388 | HMDB00870 |
| nicotinamide riboside | 1.07 | 0.9464 | | |
| L-urobilin | 1.04 | 0.9504 | C05793 | HMDB04159 |
| 1-linoleoylglycerol (1-monolinolein) | 1.02 | 0.9733 | | |
| docosahexaenoate (DHA; 22:6n3) | 0.99 | 0.9812 | C06429 | HMDB02183 |
| 10-nonadecenoate (19:1n9) | 0.95 | 0.9859 | | |
| eicosapentaenoate (EPA; 20:5n3) | 0.92 | 0.9922 | C06428 | HMDB01999 |
| 2-hydroxyglutarate | 1.36 | 0.0009 | C02630 | HMDB00606 |
| succinylcarnitine | 1.62 | 0.0017 | | |
| malonylcarnitine | 1.35 | 0.0101 | | HMDB02095 |
| glycerol | 1.27 | 0.0272 | C00116 | HMDB00131 |
| glutarate (pentanedioate) | 1.54 | 0.0403 | C00489 | HMDB00661 |
| glycocholenate sulfate | 1.04 | 0.0433 | | |
| C-glycosyltryptophan | 1.12 | 0.0734 | | |
| 3-methylglutarylcarnitine (C6) | 0.15 | 0.0823 | | HMDB00552 |
| pregnen-diol disulfate | 1.28 | 0.0989 | C05484 | HMDB04025 |
| 4-androsten-3beta,17beta-diol disulfate 1 | 1.32 | 0.1059 | | HMDB03818 |
| 2-hydroxybutyrate (AHB) | 0.91 | 0.1272 | C05984 | HMDB00008 |
| creatinine | 1.18 | 0.2356 | C00791 | HMDB00562 |
| chiro-inositol | 1.46 | 0.298 | | |
| tryptophan betaine | 1.39 | 0.3182 | C09213 | |
| 1,5-anhydroglucitol (1,5-AG) | 0.91 | 0.3416 | C07326 | HMDB02712 |
| 4-hydroxyhippurate | 0.75 | 0.591 | | |
| 4-methyl-2-oxopentanoate | 1.12 | 0.6942 | C00233 | HMDB00695 |
| glycolithocholate sulfate | 1.02 | 0.9038 | C11301 | HMDB02639 |
| N-acetylneuraminate | 1.02 | 0.9189 | C00270 | HMDB00230 |
| isoleucine | 1.43 | 3.31E−07 | C00407 | HMDB00172 |
| choline | 0.62 | 4.64E−07 | | |
| tyrosine | 1.41 | 1.32E−06 | C00082 | HMDB00158 |
| gamma-glutamylleucine | 0.65 | 1.70E-06 | | HMDB11171 |
| benzoate | 0.57 | 1.90E−06 | C00180 | HMDB01870 |
| xanthine | 1.34 | 3.64E−06 | C00385 | HMDB00292 |
| 5-methylthioadenosine (MTA) | 2.14 | 4.97E−06 | C00170 | HMDB01173 |
| N2-methylguanosine | 1.91 | 5.19E−06 | | HMDB05862 |
| fucose | 1.88 | 5.38E−06 | | HMDB00174 |
| phenylalanine | 1.4 | 5.63E−06 | C00079 | HMDB00159 |
| S-adenosylhomocysteine (SAH) | 1.72 | 5.66E−06 | C00021 | HMDB00939 |
| leucine | 1.38 | 6.36E−06 | C00123 | HMDB00687 |
| 5-oxoproline | 0.56 | 1.46E−05 | C01879 | HMDB00267 |
| citrate | 0.55 | 1.51E−05 | C00158 | HMDB00094 |
| N6-carbamoylthreonyladenosine | 1.44 | 1.93E−05 | | |
| methionine | 1.39 | 2.72E−05 | C00073 | HMDB00696 |
| adenine | 2.62 | 2.88E−05 | C00147 | HMDB00034 |
| 2-methylbutyrylcarnitine (C5) | 1.64 | 3.58E−05 | | HMDB00378 |
| xanthosine | 1.63 | 3.79E−05 | C01762 | HMDB00299 |
| pantothenate | 1.45 | 4.30E−05 | C00864 | HMDB00210 |
| gamma-glutamylvaline | 0.63 | 7.26E−05 | | HMDB11172 |
| valine | 1.28 | 7.35E−05 | C00183 | HMDB00883 |
| glycylproline | 1.42 | 7.75E−05 | | HMDB00721 |
| mannose | 1.98 | 0.0001 | C00159 | HMDB00169 |
| proline | 1.32 | 0.0001 | C00148 | HMDB00162 |
| uracil | 1.66 | 0.0002 | C00106 | HMDB00300 |
| threonine | 1.52 | 0.0002 | C00188 | HMDB00167 |
| cis-aconitate | 0.67 | 0.0002 | C00417 | HMDB00072 |
| propionylcarnitine | 1.56 | 0.0002 | C03017 | HMDB00824 |
| lactate | 1.5 | 0.0003 | C00186 | HMDB00190 |
| mannitol | 0.33 | 0.0003 | C00392 | HMDB00765 |
| hexanoylcarnitine | 1.54 | 0.0003 | C01585 | HMDB00705 |
| gamma-glutamylphenylalanine | 0.79 | 0.0004 | | HMDB00594 |
| fructose | 1.56 | 0.0005 | C00095 | HMDB00660 |
| cortisone | 1.5 | 0.0006 | C00762 | HMDB02802 |
| hypoxanthine | 1.28 | 0.0008 | C00262 | HMDB00157 |
| serine | 1.46 | 0.0009 | C00065 | HMDB03406 |
| alanine | 1.47 | 0.001 | C00041 | HMDB00161 |
| threonate | 0.59 | 0.001 | C01620 | HMDB00943 |
| acetylcarnitine | 1.31 | 0.0015 | C02571 | HMDB00201 |
| pyroglutamine | 1.63 | 0.002 | | |
| erythronate | 1.38 | 0.002 | | HMDB00613 |
| 2-isopropylmalate | 1.57 | 0.0024 | C02504 | HMDB00402 |
| gamma-glutamylisoleucine | 0.71 | 0.0026 | | HMDB11170 |
| 5,6-dihydrouracil | 2.14 | 0.0027 | C00429 | HMDB00076 |
| cysteine | 1.81 | 0.003 | C00097 | HMDB00574 |
| thymine | 1.92 | 0.0045 | C00178 | HMDB00262 |
| pseudouridine | 1.3 | 0.005 | C02067 | HMDB00767 |
| glucarate (saccharate) | 1.51 | 0.0055 | C00818 | HMDB00663 |
| xylose | 1.78 | 0.0065 | C00181 | HMDB00098 |
| glycolate (hydroxyacetate) | 0.9 | 0.0077 | C00160 | HMDB00115 |
| creatine | 1.58 | 0.008 | C00300 | HMDB00064 |
| histidine | 1.23 | 0.0082 | C00135 | HMDB00177 |
| 3-carboxy-4-methy1-5-propy1-2- | 0.58 | 0.0085 | | |
| furanpropanoate (CMPF) | | | | |
| ascorbate (Vitamin C) | 1.54 | 0.0095 | C00072 | HMDB00044 |
| pro-hydroxy-pro | 1.3 | 0.0129 | | HMDB06695 |
| succinate | 1.47 | 0.013 | C00042 | HMDB00254 |
| riboflavin (Vitamin B2) | 1.27 | 0.0147 | C00255 | HMDB00244 |
| taurine | 1.42 | 0.0221 | C00245 | HMDB00251 |
| trigonelline (N′-methylnicotinate) | 1.61 | 0.0229 | | HMDB00875 |
| glucose | 1.42 | 0.025 | C00031 | HMDB00122 |
| 3-ureidopropionate | 2.04 | 0.0267 | C02642 | HMDB00026 |
| quinate | 1.63 | 0.0299 | C00296 | HMDB03072 |
| lysine | 1.2 | 0.0307 | C00047 | HMDB00182 |
| urate | 0.83 | 0.0321 | C00366 | HMDB00289 |
| N-acetyltyrosine | 1.33 | 0.0409 | | HMDB00866 |
| Nl-methylguanosine | 1.37 | 0.0417 | | HMDB01563 |
| glucuronate | 1.46 | 0.0453 | C00191 | HMDB00127 |
| N-acetylglycine | 1.26 | 0.0502 | | HMDB00532 |
| 3-dehydrocarnitine | 1.23 | 0.0536 | | |
| tryptophan | 1.51 | 0.0574 | C00078 | HMDB00929 |
| N-6-trimethyllysine | 1.16 | 0.0679 | C03793 | HMDB01325 |
| 2-hydroxyisobutyrate | 0.88 | 0.0691 | | HMDB00729 |
| 1-methylimidazoleacetate | 0.81 | 0.0694 | C05828 | HMDB02820 |
| ribitol | 1.22 | 0.0757 | C00474 | HMDB00508 |
| isovalerylcarnitine | 1.53 | 0.0775 | | HMDB00688 |
| fumarate | 1.19 | 0.0809 | C00122 | HMDB00134 |
| sarcosine (N-Methylglycine) | 1.63 | 0.0881 | C00213 | HMDB00271 |
| N-acetylthreonine | 1.27 | 0.0945 | C01118 | |
| 2-hydroxyhippurate (salicylurate) | 1.1 | 0.0949 | C07588 | HMDB00840 |
| dimethylglycine | 1.2 | 0.0986 | C01026 | HMDB00092 |
| xylonate | 1.3 | 0.1114 | C05411 | |
| malate | 1.24 | 0.1181 | C00149 | HMDB00156 |
| alpha-hydroxyisovalerate | 1.3 | 0.1218 | | HMDB00407 |
| adenosine | 0.85 | 0.1231 | C00212 | HMDB00050 |
| beta-hydroxypyruvate | 1.11 | 0.1278 | C00168 | HMDB01352 |
| isobutyrylcarnitine | 1.28 | 0.1327 | | |
| N-acetylvaline | 1.38 | 0.1481 | | HMDB11757 |
| stachydrine | 1.52 | 0.161 | C10172 | HMDB04827 |
| nicotinate | 1.07 | 0.169 | C00253 | HMDB01488 |
| N-acetylleucine | 1.47 | 0.1865 | C02710 | HMDB11756 |
| tartarate | 1.56 | 0.2007 | C00898 | HMDB00956 |
| N6-acetyllysine | 1.15 | 0.2018 | C02727 | HMDB00206 |
| citramalate | 1.46 | 0.2034 | C00815 | HMDB00426 |
| glycine | 1.16 | 0.2096 | C00037 | HMDB00123 |
| homostachydrine | 1.57 | 0.2144 | C08283 | |
| xylulose | 1.11 | 0.2212 | C00310 | HMDB00654 |
| gulono-1,4-lactone | 1.24 | 0.2265 | C01040 | HMDB03466 |
| 2-aminobutyrate | 0.95 | 0.2316 | C02261 | HMDB00650 |
| phenylacetylglutamine | 1.3 | 0.2334 | C04148 | HMDB06344 |
| threitol | 2.91 | 0.2425 | C16884 | HMDB04136 |
| kynurenine | 1.21 | 0.2444 | C00328 | HMDB00684 |
| scyllo-inositol | 1.54 | 0.2585 | C06153 | HMDB06088 |
| N-acetylisoleucine | 1.21 | 0.2697 | | |
| guanidinoacetate | 1.57 | 0.2807 | C00581 | HMDB00128 |
| dimethylarginine (SDMA + ADMA) | 1.09 | 0.3281 | C03626 | HMDB01539 |
| | | | | HMDB03334 |
| gluconate | 1.06 | 0.3381 | C00257 | HMDB00625 |
| 5-aminovalerate | 1.22 | 0.361 | C00431 | HMDB03355 |
| 3-indoxyl sulfate | 0.87 | 0.3619 | | HMDB00682 |
| pyridoxate | 1.16 | 0.3722 | C00847 | HMDB00017 |
| cholate | 0.9 | 0.3809 | C00695 | HMDB00619 |
| sorbitol | 0.83 | 0.3962 | C00794 | HMDB00247 |
| myo-inositol | 1.27 | 0.399 | C00137 | HMDB00211 |
| androsterone sulfate | 0.89 | 0.4224 | C00523 | HMDB02759 |
| quinolinate | 1.8 | 0.4244 | C03722 | HMDB00232 |
| allo-threonine | 1.16 | 0.4274 | C05519 | HMDB04041 |
| N-acetylasparagine | 1.25 | 0.4508 | | HMDB06028 |
| gamma-aminobutyrate (GABA) | 1.2 | 0.4516 | C00334 | HMDB00112 |
| 4-guanidinobutanoate | 1.14 | 0.4601 | C01035 | HMDB03464 |
| adipate | 0.59 | 0.4795 | C06104 | HMDB00448 |
| NI-methyladenosine | 0.99 | 0.5092 | C02494 | HMDB03331 |
| N2,N2-dimethylguanosine | 1.04 | 0.513 | | HMDB04824 |
| glycerophosphorylcholine (GPC) | 0.99 | 0.5162 | C00670 | HMDB00086 |
| 2-aminoadipate | 1.01 | 0.5453 | C00956 | HMDB00510 |
| N-acetylglutamine | 1.19 | 0.5703 | C02716 | HMDB06029 |
| vanillylmandelate (VMA) | 1.22 | 0.5885 | C05584 | HMDB00291 |
| glutarylcarnitine (C5) | 1.11 | 0.6188 | | HMDB13130 |
| indolelactate | 1.18 | 0.6342 | C02043 | HMDB00671 |
| phenol sulfate | 1 | 0.6594 | C02180 | |
| N-acetyl-aspartyl-glutamate (NAAG) | 0.9 | 0.665 | C12270 | HMDB01067 |
| 3-methyl-2-oxovalerate | 1.14 | 0.681 | C00671 | HMDB03736 |
| pipecolate | 1.26 | 0.6886 | C00408 | HMDB00070 |
| 3-hydroxybutyrate (BHBA) | 1.02 | 0.6983 | C01089 | HMDB00357 |
| N-acetylphenylalanine | 1.19 | 0.7124 | C03519 | HMDB00512 |
| azelate (nonanedioate) | 0.99 | 0.7187 | C08261 | HMDB00784 |
| theobromine | 0.99 | 0.7441 | C07480 | HMDB02825 |
| glutamine | 1.02 | 0.7453 | C00064 | HMDB00641 |
| N2-acetyllysine | 1.32 | 0.7466 | C12989 | HMDB00446 |
| indoleacetate | 0.92 | 0.7704 | C00954 | HMDB00197 |
| 3-methylhistidine | 0.97 | 0.7855 | C01152 | HMDB00479 |
| N-acetylarginine | 1.45 | 0.7887 | C02562 | HMDB04620 |
| octanoylcarnitine | 1.18 | 0.796 | | |
| 3-aminoisobutyrate | 1.21 | 0.8027 | C05145 | HMDB03911 |
| trans-urocanate | 1 | 0.8589 | C00785 | HMDB00301 |
| catechol sulfate | 0.79 | 0.8966 | C00090 | |
| 4-hydroxyphenylacetate | 1.01 | 0.8992 | C00642 | HMDB00020 |
| p-cresol sulfate | 1.05 | 0.9092 | C01468 | |
| glycerol 3-phosphate (G3P) | 1.03 | 0.9262 | C00093 | HMDB00126 |
| hippurate | 0.8 | 0.9285 | C01586 | HMDB00714 |
| anserine | 0.97 | 0.9341 | C01262 | HMDB00194 |
| aspartate | 1.03 | 0.9454 | C00049 | HMDB00191 |
| N-acetylaspartate (NAA) | 0.97 | 0.9552 | C01042 | HMDB00812 |
| carnitine | 1.01 | 0.9555 | | |
| beta-alanine | 1.15 | 0.9745 | C00099 | HMDB00056 |
| glutamate | 0.99 | 0.9867 | C00025 | HMDB03339 |

The biomarkers were used to create a statistical model to classify the subjects. The biomarkers were evaluated using Random Forest analysis to classify subjects as having low stage or high stage kidney cancer. Samples from 56 subjects with Low stage RCC (T1, T2) and 84 subjects with High stage RCC (T3,T4) were used in this analysis.

Random Forest results show that the samples were classified with 72% prediction accuracy. The Confusion Matrix presented in Table 9 shows the number of samples predicted for each classification and the actual in each group (Low Stage or High Stage). The “Out-of-Bag” (OOB) Error rate gives an estimate of how accurately new observations can be predicted using the Random Forest model (e.g., whether a sample is from a subject with low stage RCC or high stage RCC). The OOB error from this Random Forest was approximately 28%, and the model estimated that, when used on a new set of subjects, the identity of low stage RCC subjects could be predicted correctly 68% of the time and high stage RCC subjects could be predicted 75% of the time.

TABLE 9
Results of Random Forest: Low Stage vs. High Stage RCC

| | | Predicted Group | | |
|---|---|---|---|---|
| | | Low | High | Class |
| | | Stage | Stage | Error |
| Actual | Low | 38 | 18 | 0.3214 |
| Group | Stage | | | |
| | High | 21 | 63 | 0.25 |
| | Stage | | | |

Predictive accuracy = 72%

Based on the OOB Error rate of 28%, the Random Forest model that was created predicted whether a sample was from an individual with low stage or high stage kidney cancer with about 72% accuracy based on the levels of the biomarkers measured in samples from the subject. Exemplary biomarkers for distinguishing the groups are choline, pelargonate (9:0), arginine, gamma-glutamylleucine, xanthine, tyrosine, 5-oxoproline, inositol-1-phosphate (11P), N2-methylguanosine, isoleucine, 2-ethylhexanoate, leucine, adenine, 5-methylthioadenosine (MTA), laurate (12:0), phenylalanine, mannose, uracil, xanthosine, erythritol, guanosine-5-monophosphate-5 (GMP), homocysteine, lactate, 4-hydroxybutyrate (GHB), ribose, fucose, S-adenosylhomocysteine (SAH), mannitol, hypoxanthine, and threonine.

The Random Forest analysis demonstrated that by using the biomarkers, low stage kidney cancer subjects were distinguished from high stage kidney cancer subjects with 75% sensitivity, 68% specificity, 78% PPV and 64% NPV.

### Example 5

### Tissue Biomarkers for Kidney Cancer Aggressiveness

Tumors from subjects with kidney cancer were assessed for aggressiveness based on three criteria: tumor stage, tumor grade, and tumor metastatic potential. To identify biomarkers of kidney cancer aggressiveness, metabolomic analysis was carried out on tissue samples from 140 subjects with kidney cancer. Tumor stage, grade and metastatic potential were reported for each subject. After the levels of metabolites were determined, the data were analyzed using a mixed model that consists of fixed effects and a random effect. Fisher's method was then used combine the aggressiveness criteria of tumor stage, tumor grade and tumor metastatic potential to identify biomarkers that are associated with kidney cancer aggressiveness. The 50 biomarkers most highly associated with kidney cancer aggressiveness are listed in Table 10.

Table 10 includes, for each biomarker, the biochemical name of the biomarker, the internal identifier for that biomarker compound in the in-house chemical library of authentic standards (CompID), the p-value determined in the statistical analysis of the data concerning the biomarkers, and whether the biomarker is positively or negatively associated with aggressiveness. A positive association means that as kidney cancer aggressiveness increases, the level of the biomarker increases (i.e., the biomarker is higher in more aggressive cancer); a negative association means that as kidney cancer aggressiveness increases, the level of the biomarker decreases (i.e., the biomarker is lower in more aggressive cancer).

TABLE 10
Tissue Biomarkers for Kidney Cancer Aggressiveness

| | | | Aggressiveness |
|---|---|---|---|
| Biochemical Name | CompID | P-value | Association |
| pelargonate (9:0) | 12035 | 1.75E−13 | negative |
| laurate (12:0) | 1645 | 5.59E−12 | negative |
| homocysteine | 40266 | 1.63E−09 | positive |
| 2′-deoxyinosine | 15076 | 2.48E−09 | positive |
| S-adenosylmethionine (SAM) | 15915 | 2.49E−09 | positive |
| glycylthreonine | 42050 | 3.72E−09 | positive |
| aspartylphenylalanine | 22175 | 4.05E−09 | positive |
| phenylalanylglycine | 41370 | 4.63E−09 | positive |
| cytidine 5′-diphosphocholine | 34418 | 2.02E−08 | positive |
| alanylglycine | 37075 | 3.69E−08 | positive |
| lysylmethionine | 41943 | 4.41E−08 | positive |
| glycylisoleucine | 36659 | 4.87E−08 | positive |
| ribose | 12080 | 5.25E−08 | positive |
| aspartylleucine | 40068 | 5.66E−08 | positive |
| 2-ethylhexanoate | 1554 | 6.27E−08 | negative |
| asparagine | 11398 | 7.16E−08 | positive |
| homoserine | 23642 | 9.90E−08 | positive |
| 2′-deoxyguanosine | 1411 | 2.69E−07 | positive |
| valerylcarnitine | 34406 | 3.06E−07 | positive |
| 4-hydroxybutyrate (GHB) | 34585 | 5.40E−07 | positive |
| caprate (10:0) | 1642 | 7.22E−07 | negative |
| galactose | 12055 | 8.03E−07 | positive |
| heme | 41754 | 1.06E−06 | negative |
| butyrylcarnitine | 32412 | 1.07E−06 | positive |
| choline | 15506 | p < 0.000001 | negative |
| isoleucine | 1125 | 2.20E−13 | positive |
| mannitol | 15335 | 7.67E−13 | negative |
| fucose | 15821 | 2.92E−11 | positive |
| tyrosine | 1299 | 2.03E−10 | positive |
| xanthine | 3147 | 5.42E−10 | positive |
| 5-oxoproline | 1494 | 1.34E−09 | negative |
| 5-methylthioadenosine (MTA) | 1419 | 1.59E−09 | positive |
| phenylalanine | 64 | 2.02E−09 | positive |
| leucine | 60 | 2.08E−09 | positive |
| threonate | 27738 | 2.16E−09 | negative |
| gamma-glutamylleucine | 18369 | 4.43E−09 | negative |
| benzoate | 15778 | 6.98E−09 | negative |
| proline | 1898 | 8.66E−09 | positive |
| methionine | 1302 | 1.44E−08 | positive |
| glycylproline | 22171 | 2.31E−08 | positive |
| N2-methylguanosine | 35133 | 2.77E−08 | positive |
| adenine | 554 | 4.62E−08 | positive |
| 2-methylbutyroylcarnitine | 35431 | 5.90E−08 | positive |
| S-adenosylhomocysteine | 15948 | 6.07E−08 | positive |
| (SAH) | | | |
| citrate | 1564 | 6.61E−08 | negative |
| xanthosine | 15136 | 1.43E−07 | positive |
| 5,6-dihydrouracil | 1559 | 3.42E−07 | positive |
| threonine | 1284 | 5.28E−07 | positive |
| valine | 1649 | 5.84E−07 | positive |
| pantothenate | 1508 | 7.64E−07 | positive |

### VII. Example 6

### Urine Biomarkers for Renal Cell Carcinoma

To identify biomarkers of renal cell carcinoma, urine samples collected

from subjects with: 1) RCC, 2) prostate cancer (PCA), 3) bladder cancer (BCA) and 4) normal subjects were analyzed metabolomically. After the levels of metabolites were determined, biomarkers of RCC were identified using one-way ANOVA contrasts. Biomarkers of RCC were identified as metabolites that differed between 1) RCC and normal subjects, 2) RCC and PCA subjects, and/or 3) RCC and BCA subjects. The biomarkers are listed in Table 11.

Table 11 includes, for each biomarker, the biochemical name of the biomarker, the fold change (FC) of the biomarker in 1) RCC compared to Normal, 2) RCC compared to BCA, 3) RCC compared to PCA, and the p-value determined in the statistical analysis of the data concerning the biomarkers. In column 8 of Table 11, the identifier for that biomarker compound in the Human Metabolome Database (HMDB), if available, is listed. Bold values indicate a fold of change with a p-value of <0.1.

TABLE 11
Urine biomarkers for kidney cancer

| | RCC/Norm | | RCC/BCA | | RCC/PCA | | |
|---|---|---|---|---|---|---|---|
| Biochemical Name | FC | P-value | FC | P-value | FC | P-value | HMDB |
| 3-hydroxyhippurate | 0.32 | 7.35E−11 | 0.79 | 0.8623 | 1.91 | 0.6142 | HMDB06116 |
| methyl indole-3-acetate | 5.91 | 7.93E−12 | 4.36 | 4.23E−09 | 1.82 | 0.3269 | |
| 2,3-dihydroxyisovalerate | 0.14 | 9.50E−11 | 0.52 | 0.1943 | 0.78 | 0.4462 | |
| cinnamoylglycine | 0.39 | 1.31E−08 | 0.8 | 0.2802 | 1.18 | 0.1474 | |
| galactose | 0.45 | 4.18E−08 | 0.67 | 0.0026 | 0.89 | 0.0022 | HMDB00143 |
| 4-hydroxy-2-oxoglutaric acid | 4.71 | 5.90E−08 | 1.76 | 0.0349 | 0.99 | 0.2168 | HMDB02070 |
| gluconate | 12.15 | 1.05E−07 | 1.1 | 0.6536 | 0.49 | 7.27E−12 | HMDB00625 |
| 1,2-propanediol | 3.15 | 1.86E−07 | 0.59 | 0.5991 | 0.14 | 5.08E−05 | HMDB01881 |
| 2-oxindole-3-acetate | 0.42 | 2.33E−07 | 0.91 | 0.3503 | 2.16 | 0.0005 | |
| alpha-CEHC glucuronide | 0.37 | 6.71E−07 | 0.79 | 0.8128 | 1.41 | 0.0215 | |
| ethanolamine | 0.57 | 9.18E−07 | 0.87 | 0.0147 | 1.02 | 0.1873 | HMDB00149 |
| phenylpropionylglycine | 0.42 | 9.40E−07 | 0.84 | 0.5281 | 0.86 | 0.7559 | HMDB00860 |
| 2,3-butanediol | 0.26 | 1.72E−06 | 0.6 | 0.0055 | 0.63 | 0.0068 | HMDB03156 |
| adenosine 5′-monophosphate | 3.23 | 4.40E−06 | 0.15 | 0.0019 | 0.59 | 0.0005 | HMDB00045 |
| (AMP) | | | | | | | |
| N6-methyladenosine | 2.49 | 5.48E−06 | 1.48 | 0.0046 | 1.18 | 0.5508 | HMDB04044 |
| caffeate | 0.39 | 9.78E−05 | 0.47 | 0.0019 | 0.98 | 0.3662 | HMDB01964 |
| 1-(3-aminopropyl)-2- | 1.6 | 0.0003 | 1 | 0.5363 | 1.78 | 9.44E−05 | |
| pyrrolidone | | | | | | | |
| gamma-CEHC | 1.67 | 0.0017 | 2.68 | 5.11E−06 | 1.64 | 0.0154 | HMDB01931 |
| 21-hydroxypregnenolone | 1.35 | 0.0067 | 1.7 | 0.0013 | 1.26 | 0.4325 | HMDB04026 |
| disulfate | | | | | | | |
| guanine | 1.02 | 0.1408 | 1.08 | 0.7162 | 0.68 | 0.0001 | HMDB00132 |
| sulforaphane | 1.09 | 0.2226 | 1.28 | 0.0849 | 1.52 | 0.0284 | HMDB05792 |
| imidazole propionate | 1.19 | 0.2819 | 0.85 | 0.0028 | 2 | 0.2612 | HMDB02271 |
| 12-dehydrocholate | 2.31 | 0.2856 | 2.67 | 0.0266 | 4.26 | 0.0008 | HMDB00400 |
| 3-sialyllactose | 1.34 | 0.3463 | 1.5 | 0.0239 | 1.79 | 0.0013 | HMDB00825 |
| Isobar: glucuronate, | 0.85 | 0.4657 | 0.96 | 0.6749 | 1.46 | 0.0002 | |
| galacturonate, 5-keto-gluconate | | | | | | | |
| N-methyl proline | 0.77 | 0.5755 | 0.48 | 0.0034 | 0.84 | 0.5548 | |
| orotidine | 1.06 | 0.7045 | 0.67 | 0.7869 | 1.73 | 0.0067 | HMDB00788 |
| palmitoyl sphingomyelin | 2.7 | 0.839 | 0.26 | 0.0001 | 2.3 | 0.4001 | |
| methyl-4-hydroxybenzoate | 29.08 | p < 0.0001 | 3.87 | 3.94E−07 | 1.19 | 0.0499 | |
| 2,5-furandicarboxylic acid | 0.39 | 5.05E−07 | 0.69 | 0.1772 | 2.16 | 0.0681 | HMDB04812 |
| arginine | 0.23 | 8.65E−07 | 0.6 | 0.0463 | 1.16 | 0.5876 | HMDB00517 |
| homoserine | 0.47 | 5.06E−06 | 0.51 | 0.0383 | 0.89 | 0.5568 | HMDB00719 |
| N-acetyltryptophan | 0.43 | 5.93E−06 | 0.89 | 0.2169 | 1.74 | 0.0287 | |
| cyclo(leu-pro) | 0.52 | 1.15E−05 | 0.53 | 0.0025 | 0.96 | 0.5245 | |
| 2,4,6-trihydroxybenzoate | 0.24 | 2.47E−05 | 0.65 | 0.4021 | 1.29 | 0.8021 | |
| 3-hydroxyproline | 0.74 | 6.60E−05 | 0.92 | 0.0356 | 1.04 | 0.3894 | |
| putrescine | 0.4 | 7.27E−05 | 0.33 | 0.0854 | 1.47 | 0.202 | HMDB01414 |
| cortisol | 2.21 | 8.35E−05 | 0.85 | 0.3051 | 0.89 | 0.1558 | HMDB00063 |
| N-acetylcysteine | 0.45 | 8.79E−05 | 0.68 | 0.1831 | 0.82 | 0.5203 | HMDB01890 |
| pinitol | 0.23 | 0.0001 | 0.28 | 0.0339 | 1.14 | 0.9708 | |
| N-carbamoylsarcosine | 0.72 | 0.0001 | 0.84 | 0.1691 | 1.32 | 0.2097 | |
| 2-methylhippurate | 1.67 | 0.0001 | 0.58 | 0.8307 | 1.14 | 0.6518 | HMDB11723 |
| dihydroferulic acid | 0.28 | 0.0002 | 0.38 | 0.1143 | 0.72 | 0.6212 | |
| 3-hydroxybenzoate | 0.62 | 0.0002 | 0.79 | 0.0647 | 1.14 | 0.5684 | HMDB02466 |
| ethyl glucuronide | 0.34 | 0.0003 | 1.43 | 0.0816 | 1.71 | 0.7613 | |
| ciliatine (2- | 0.37 | 0.0003 | 0.19 | 0.33 | 0.56 | 0.719 | HMDB11747 |
| aminoethylphosphonate) | | | | | | | |
| 3-phosphoglycerate | 0.68 | 0.0004 | 0.65 | 0.4871 | 1.31 | 0.4863 | HMDB00807 |
| inosine | 1.69 | 0.0004 | 1.17 | 0.0139 | 1.38 | 0.0445 | |
| 3-methylglutaconate | 0.69 | 0.0005 | 0.87 | 0.3421 | 0.9 | 0.2874 | HMDB00522 |
| alanylalanine | 0.59 | 0.0008 | 0.8 | 0.3922 | 0.8 | 0.6212 | HMDB03459 |
| 5-methyltetrahydrofolate | 0.35 | 0.001 | 0.79 | 0.5757 | 0.63 | 0.1217 | HMDB01396 |
| (5MeTHF) | | | | | | | |
| galactinol | 0.48 | 0.0012 | 1.02 | 0.9326 | 1.37 | 0.1909 | HMDB05826 |
| trans-aconitate | 0.73 | 0.0012 | 0.95 | 0.4419 | 0.95 | 0.3384 | HMDB00958 |
| dopamine | 0.53 | 0.0017 | 0.93 | 0.5238 | 1.18 | 0.4495 | HMDB00073 |
| guanidine | 0.6 | 0.0024 | 1.2 | 0.3713 | 1.08 | 0.9767 | HMDB01842 |
| 3-hydroxymandelate | 0.32 | 0.0032 | 1.49 | 0.3071 | 2.88 | 0.9955 | HMDB00750 |
| asparagine | 0.68 | 0.0034 | 0.81 | 0.2918 | 1.05 | 0.1835 | HMDB00168 |
| 2-phenylglycine | 0.7 | 0.0034 | 0.43 | 0.19 | 0.25 | 0.7127 | HMDB02210 |
| S-methylcysteine | 0.74 | 0.0036 | 0.8 | 0.1326 | 0.79 | 0.3376 | HMDB02108 |
| 2-pyrrolidinone | 0.64 | 0.0043 | 1.12 | 0.6896 | 0.97 | 0.5848 | HMDB02039 |
| N-acetylproline | 0.68 | 0.0044 | 0.97 | 0.964 | 1.08 | 0.9559 | |
| L-urobilin | 1 | 0.0044 | 1.31 | 0.4793 | 2 | 0.6431 | HMDB04159 |
| abscisate | 0.38 | 0.0054 | 0.65 | 0.4202 | 1.08 | 0.8488 | |
| N-acetyl-beta-alanine | 0.76 | 0.0054 | 0.8 | 0.0741 | 0.82 | 0.0814 | |
| N-acetylserine | 1.43 | 0.0054 | 0.97 | 0.9362 | 1.32 | 0.0554 | HMDB02931 |
| cystine | 0.54 | 0.0059 | 1.57 | 0.4268 | 0.95 | 0.8388 | HMDB00192 |
| N-methylglutamate | 0.68 | 0.0059 | 0.7 | 0.9942 | 1.24 | 0.1644 | |
| arabonate | 0.77 | 0.0066 | 0.92 | 0.4588 | 1.05 | 0.9858 | HMDB00539 |
| glycodeoxycholate | 0.62 | 0.0075 | 0.56 | 0.0348 | 1.44 | 0.9653 | HMDB00631 |
| phosphoethanolamine | 1.04 | 0.008 | 1.24 | 0.5162 | 2.52 | 0.2976 | HMDB00224 |
| 5alpha-pregnan-3beta,20alpha- | 2.24 | 0.0082 | 2.55 | 0.0051 | 2.07 | 0.1394 | |
| diol disulfate | | | | | | | |
| alpha-tocopherol | 4.01 | 0.0082 | 0.65 | 0.0484 | 3.03 | 0.0997 | HMDB01893 |
| N-carbamoylaspartate | 0.38 | 0.0093 | 0.88 | 0.8658 | 1.06 | 0.4614 | HMDB00828 |
| aspartylaspartate | 0.79 | 0.012 | 1.35 | 0.9659 | 1.06 | 0.6221 | |
| 2-octenedioate | 0.7 | 0.0121 | 0.92 | 0.5898 | 0.56 | 0.3035 | HMDB00341 |
| 2-(4-hydroxyphenyl)propionate | 0.4 | 0.0125 | 1.01 | 0.4775 | 4.01 | 0.8379 | |
| 6-sialyl-N-acetyllactosamine | 1.33 | 0.0138 | 1.4 | 0.0132 | 1.55 | 0.0005 | HMDB06584 |
| diglycerol | 0.69 | 0.014 | 0.75 | 0.128 | 1.16 | 0.7456 | |
| biotin | 0.56 | 0.0157 | 1.12 | 0.549 | 1.44 | 0.4336 | HMDB00030 |
| pyridoxal | 0.5 | 0.0167 | 1.24 | 0.2877 | 1.71 | 0.0158 | HMDB01545 |
| pyridoxine (Vitamin B6) | 0.43 | 0.019 | 1 | 1 | 1 | 1 | HMDB02075 |
| daidzein | 0.64 | 0.024 | 0.71 | 0.3 | 0.94 | 0.882 | HMDB03312 |
| pregnanediol-3-glucuronide | 1.8 | 0.024 | 2 | 0.0328 | 1.46 | 0.939 | |
| Isobar: dihydrocaffeate, 3,4- | 0.74 | 0.0244 | 0.72 | 0.1813 | 1.26 | 0.9461 | |
| dihydroxycinnamate | | | | | | | |
| guanosine | 1.32 | 0.0282 | 1.15 | 0.1707 | 1.57 | 0.006 | HMDB00133 |
| 3-hydroxyglutarate | 0.78 | 0.0327 | 1.11 | 0.6713 | 0.99 | 0.3518 | HMDB00428 |
| N1-Methyl-2-pyridone-5- | 0.75 | 0.0421 | 0.82 | 0.8673 | 1.1 | 0.2268 | HMDB04193 |
| carboxamide | | | | | | | |
| 5alpha-androstan-3beta,17beta- | 1.49 | 0.0491 | 1.69 | 0.0091 | 0.97 | 0.6298 | HMDB00493 |
| diol disulfate | | | | | | | |
| sinapate | 0.5 | 0.0504 | 0.79 | 0.6032 | 1.26 | 0.6029 | |
| 2-oxo-1-pyrrolidinepropionate | 1 | 0.0609 | 0.92 | 0.575 | 1.68 | 0.0135 | |
| citraconate | 0.67 | 0.062 | 0.75 | 0.1805 | 0.64 | 0.0883 | HMDB00634 |
| glucose | 0.2 | 0.0626 | 0.48 | 0.4248 | 1.36 | 0.3522 | HMDB00122 |
| glucono-1,5-lactone | 4.62 | 0.0656 | 0.54 | 0.0246 | 0.41 | 0.0003 | HMDB00150 |
| nicotinamide | 0.61 | 0.0728 | 0.48 | 0.1121 | 0.93 | 0.8341 | HMDB01406 |
| arabitol | 0.82 | 0.073 | 0.98 | 0.9546 | 0.97 | 0.7759 | HMDB01851 |
| prolylglycine | 0.81 | 0.0767 | 0.92 | 0.608 | 1.29 | 0.5811 | |
| 3-(4-hydroxyphenyl)lactate | 0.95 | 0.0789 | 1.28 | 0.9833 | 2.77 | 0.0561 | HMDB00755 |
| 5alpha-pregnan-3alpha,20beta- | 1.73 | 0.0804 | 1.83 | 0.024 | 2.1 | 0.0132 | |
| diol disulfate 1 | | | | | | | |
| sulforaphane-N-acetyl-cysteine | 0.77 | 0.0822 | 0.97 | 0.8418 | 0.97 | 0.8452 | |
| ethylmalonate | 1.17 | 0.0844 | 1.1 | 0.3975 | 0.99 | 0.7187 | HMDB00622 |
| hydantoin-5-propionic acid | 1.34 | 0.0964 | 1.38 | 0.1544 | 1.37 | 0.1151 | HMDB01212 |
| 3-hydroxycinnamate (m- | 0.58 | 0.0968 | 0.89 | 0.7784 | 1.18 | 0.6958 | HMDB01713 |
| coumarate) | | | | | | | |
| glucose-6-phosphate (G6P) | 1 | 0.2504 | 0.59 | 0.0028 | 1.42 | 0.8295 | HMDB01401 |
| glutathione, reduced (GSH) | 0.92 | 0.333 | 0.13 | 0.0003 | 0.79 | 0.5709 | HMDB00125 |
| prostaglandin E2 | 0.98 | 0.7664 | 0.71 | 0.0016 | 0.83 | 0.365 | HMDB01220 |
| biliverdin | 1 | 1 | 0.83 | 0.0016 | 0.98 | 0.6548 | HMDB01008 |
| glycerol | 12.19 | 1.70E−12 | 3.19 | 6.57E−06 | 0.73 | 0.5371 | HMDB00131 |
| pregnen-diol disulfate | 1.74 | 3.82E−05 | 1.7 | 0.0165 | 1.41 | 0.7439 | HMDB04025 |
| 4-androsten-3beta,17beta-diol | 1.63 | 0.0007 | 1.69 | 0.0015 | 1.09 | 0.5963 | HMDB03818 |
| disulfate 1 | | | | | | | |
| 1,3-dimethylurate | 0.64 | 0.0009 | 0.62 | 0.0195 | 0.84 | 0.0069 | HMDB01857 |
| 2-hydroxybutyrate (AHB) | 1.86 | 0.003 | 0.63 | 0.2777 | 0.28 | 0.0014 | HMDB00008 |
| 4-androsten-3beta,17beta-diol | 1.47 | 0.0038 | 1.81 | 0.0016 | 1.1 | 0.8567 | HMDB03818 |
| disulfate 2 | | | | | | | |
| 4-methyl-2-oxopentanoate | 1.59 | 0.0066 | 0.95 | 0.6361 | 0.75 | 0.4842 | HMDB00695 |
| UDP-glucuronate | 0.79 | 0.0262 | 0.91 | 0.6583 | 1.18 | 0.2571 | HMDB00935 |
| andro steroid monosulfate 2 | 1.96 | 0.0303 | 2.09 | 0.0528 | 1.44 | 0.6911 | HMDB02759 |
| C-glycosyltryptophan | 1.29 | 0.0392 | 1.27 | 0.0251 | 1.33 | 0.0158 | |
| andro steroid monosulfate 1 | 1.4 | 0.0411 | 1.37 | 0.0722 | 0.92 | 0.6729 | HMDB02759 |
| sucralose | 0.46 | 0.0548 | 1.13 | 0.6182 | 1.17 | 0.6149 | |
| glycocholenate sulfate | 1.52 | 0.0589 | 1.74 | 0.0684 | 1.27 | 0.552 | |
| 2-hydroxyglutarate | 1.66 | 0.067 | 1.72 | 0.0173 | 1.31 | 0.9778 | HMDB00606 |
| oxalate (ethanedioate) | 2.03 | 0.0681 | 0.96 | 0.9104 | 1.81 | 0.1906 | HMDB02329 |
| methylglutaroylcarnitine | 0.75 | 0.0965 | 0.81 | 0.3529 | 0.97 | 0.9447 | HMDB00552 |
| 4-hydroxyhippurate | 1.26 | 0.1096 | 1.64 | 0.163 | 2.56 | 0.0004 | |
| catechol sulfate | 0.3 | p < 0.0001 | 0.46 | 0.0011 | 0.73 | 0.2137 | |
| N-(2-furoyl)glycine | 0.15 | 9.50E−14 | 0.29 | 0.0003 | 0.63 | 0.203 | HMDB00439 |
| 2-hydroxyhippurate | 0.04 | 1.18E−12 | 0.29 | 0.4502 | 0.97 | 0.648 | HMDB00840 |
| (salicylurate) | | | | | | | |
| 3-hydroxyphenylacetate | 0.21 | 3.08E−12 | 0.75 | 0.7979 | 0.66 | 0.3209 | HMDB00440 |
| 2-isopropylmalate | 0.19 | 2.43E−11 | 0.63 | 0.2479 | 1.35 | 0.8165 | HMDB00402 |
| phenylacetylglycine | 0.39 | 5.98E−10 | 0.68 | 0.0045 | 2.06 | 0.0436 | HMDB00821 |
| sorbose | 0.22 | 2.34E−09 | 0.37 | 0.0572 | 0.7 | 0.5234 | HMDB01266 |
| sucrose | 0.4 | 9.07E−09 | 0.88 | 0.0023 | 1.63 | 0.193 | HMDB00258 |
| 3-hydroxypyridine | 0.36 | 1.90E−08 | 0.5 | 0.0009 | 1.01 | 0.6845 | |
| 1,3,7-trimethylurate | 0.33 | 6.47E−08 | 0.49 | 0.0017 | 0.94 | 0.0256 | HMDB02123 |
| hexanoylglycine | 1.94 | 1.23E−07 | 1.2 | 0.1663 | 0.71 | 0.0342 | HMDB00701 |
| vanillate | 0.31 | 2.49E−07 | 0.32 | 0.0079 | 1.17 | 0.778 | HMDB00484 |
| 3,4-dihydroxyphenylacetate | 0.45 | 5.32E−07 | 0.97 | 0.4211 | 0.89 | 0.0458 | HMDB01336 |
| tartarate | 0.08 | 9.57E−07 | 0.31 | 0.5399 | 0.79 | 0.3541 | HMDB00956 |
| theobromine | 0.4 | 1.39E−06 | 0.63 | 0.0275 | 0.78 | 0.0477 | HMDB02825 |
| adipate | 5.03 | 1.71E−06 | 1.11 | 0.4498 | 1.46 | 0.6544 | HMDB00448 |
| riboflavin (Vitamin B2) | 0.26 | 2.75E−06 | 1.05 | 0.189 | 1.01 | 0.346 | HMDB00244 |
| allo-threonine | 0.63 | 3.90E−06 | 0.93 | 0.055 | 0.85 | 0.8116 | HMDB04041 |
| caffeine | 0.23 | 3.96E−06 | 0.34 | 0.003 | 0.74 | 0.1958 | HMDB01847 |
| 2-aminoadipate | 0.62 | 5.33E−06 | 0.96 | 0.0542 | 0.96 | 0.5549 | HMDB00510 |
| 5-aminovalerate | 0.48 | 5.79E−06 | 0.31 | 0.1099 | 1.01 | 0.9767 | HMDB03355 |
| 5-methylthioadenosine (MTA) | 2.18 | 6.44E−06 | 2.04 | 0.0002 | 1.33 | 0.2644 | HMDB01173 |
| isobutyrylcarnitine | 0.56 | 6.56E−06 | 0.73 | 0.3009 | 0.84 | 0.5299 | |
| xanthurenate | 0.68 | 9.84E−06 | 1.17 | 0.2871 | 1.08 | 0.5768 | HMDB00881 |
| scyllo-inositol | 0.47 | 1.10E−05 | 0.59 | 0.0395 | 0.87 | 0.6725 | HMDB06088 |
| fructose | 0.4 | 1.33E−05 | 0.72 | 0.7677 | 1.17 | 0.1565 | HMDB00660 |
| 4-hydroxymandelate | 0.56 | 1.34E−05 | 0.78 | 0.4183 | 0.82 | 0.0552 | HMDB00822 |
| p-cresol sulfate | 0.6 | 1.51E−05 | 1.23 | 0.1282 | 1.33 | 0.1905 | |
| nicotinate | 0.49 | 2.82E−05 | 0.58 | 0.0062 | 1.17 | 0.9441 | HMDB01488 |
| tyramine | 0.62 | 3.42E−05 | 0.91 | 0.9143 | 0.86 | 0.2212 | HMDB00306 |
| 5-acetylamino-6-formylamino- | 0.61 | 3.46E−05 | 0.84 | 0.1381 | 1.24 | 0.0472 | HMDB11105 |
| 3-methyluracil | | | | | | | |
| 3-(3-hydroxyphenyl)propionate | 0.25 | 3.48E−05 | 0.53 | 0.3567 | 1.6 | 0.6808 | HMDB00375 |
| 1-methylxanthine | 0.46 | 3.79E−05 | 0.42 | 0.0247 | 0.63 | 0.0115 | |
| trigonelline (N′- | 0.67 | 4.67E−05 | 0.68 | 0.0012 | 1.28 | 0.4077 | HMDB00875 |
| methylnicotinate) | | | | | | | |
| 3-methylxanthine | 0.47 | 4.98E−05 | 0.76 | 0.1971 | 0.86 | 0.1676 | HMDB01886 |
| glucosamine | 0.45 | 5.50E−05 | 0.99 | 0.2774 | 1.35 | 0.3249 | HMDB01514 |
| 1,6-anhydroglucose | 0.48 | 5.55E−05 | 0.71 | 0.1691 | 1 | 0.2081 | HMDB00640 |
| 3-methylcrotonylglycine | 0.65 | 5.67E−05 | 1.1 | 0.402 | 1.56 | 0.2008 | HMDB00459 |
| gulono-1,4-lactone | 2.04 | 5.93E−05 | 1.09 | 0.2409 | 0.66 | 0.0003 | HMDB03466 |
| quinate | 0.66 | 7.93E−05 | 0.81 | 0.0009 | 0.94 | 0.0002 | HMDB03072 |
| mesaconate (methylfumarate) | 0.62 | 8.49E−05 | 0.99 | 0.3644 | 1.08 | 0.5564 | HMDB00749 |
| sebacate (decanedioate) | 2.53 | 0.0001 | 0.62 | 0.1849 | 0.51 | 0.4858 | HMDB00792 |
| N-acetylphenylalanine | 0.65 | 0.0001 | 1.1 | 0.7182 | 1.93 | 0.0012 | HMDB00512 |
| beta-alanine | 0.32 | 0.0002 | 0.5 | 0.0008 | 1.47 | 0.3724 | HMDB00056 |
| 3-hydroxybutyrate (BHBA) | 5.92 | 0.0002 | 0.31 | 0.1711 | 0.09 | 0.0007 | HMDB00357 |
| alanine | 0.72 | 0.0002 | 0.78 | 0.015 | 1.32 | 0.0133 | HMDB00161 |
| sarcosine (N-Methylglycine) | 0.76 | 0.0002 | 0.96 | 0.0758 | 1.32 | 0.3949 | HMDB00271 |
| 3-methyl-2-oxovalerate | 1.71 | 0.0002 | 1.04 | 0.2866 | 0.67 | 0.3559 | HMDB03736 |
| 1-methylhistidine | 0.55 | 0.0002 | 1 | 0.6429 | 0.88 | 0.1937 | HMDB00001 |
| 1,7-dimethylurate | 0.62 | 0.0002 | 0.74 | 0.1286 | 0.85 | 0.0177 | HMDB11103 |
| isobutyrylglycine | 0.77 | 0.0002 | 1.25 | 0.2172 | 1.61 | 0.1927 | HMDB00730 |
| cortisone | 1.33 | 0.0004 | 0.99 | 0.9786 | 1.08 | 0.9413 | HMDB02802 |
| methionine | 0.71 | 0.0005 | 0.83 | 0.0273 | 0.99 | 0.9993 | HMDB00696 |
| gamma-aminobutyrate (GABA) | 0.52 | 0.0005 | 0.95 | 0.7208 | 1.11 | 0.4535 | HMDB00112 |
| anserine | 0.34 | 0.0005 | 1.44 | 0.5487 | 2.75 | 0.4523 | HMDB00194 |
| hippurate | 0.72 | 0.0006 | 0.74 | 0.0318 | 0.91 | 0.0576 | HMDB00714 |
| tryptophan | 1.53 | 0.0008 | 1.16 | 0.5013 | 1.1 | 0.6423 | HMDB00929 |
| hexanoylcarnitine | 1.43 | 0.0008 | 1.18 | 0.1281 | 1 | 0.8835 | HMDB00705 |
| phenyllactate (PLA) | 0.42 | 0.0009 | 0.72 | 0.0623 | 1.61 | 0.6146 | HMDB00779 |
| paraxanthine | 0.49 | 0.001 | 0.38 | 0.0028 | 0.59 | 0.0092 | HMDB01860 |
| pyridoxate | 0.36 | 0.0011 | 1.1 | 0.683 | 1.02 | 0.773 | HMDB00017 |
| arabinose | 0.72 | 0.0012 | 0.84 | 0.0726 | 0.91 | 0.0854 | HMDB00646 |
| 7-methylxanthine | 0.53 | 0.0012 | 0.77 | 0.2641 | 0.87 | 0.4015 | HMDB01991 |
| 7-methylguanine | 1.29 | 0.0012 | 1.06 | 0.7499 | 1.16 | 0.2737 | HMDB00897 |
| decanoylcarnitine | 1.65 | 0.0015 | 1.58 | 0.0313 | 0.91 | 0.2273 | HMDB00651 |
| ascorbate (Vitamin C) | 0.13 | 0.0017 | 0.54 | 0.2485 | 0.86 | 0.0675 | HMDB00044 |
| acetylcarnitine | 1.95 | 0.0019 | 0.82 | 0.3328 | 0.68 | 0.0232 | HMDB00201 |
| lysine | 0.66 | 0.002 | 1.02 | 0.2246 | 1.17 | 0.2675 | HMDB00182 |
| guanidinoacetate | 0.73 | 0.002 | 1.17 | 0.99 | 1.62 | 0.5165 | HMDB00128 |
| phenylacetylglutamine | 0.81 | 0.0022 | 1.14 | 0.0032 | 1.46 | 0.006 | HMDB06344 |
| itaconate (methylenesuccinate) | 0.81 | 0.0028 | 1.38 | 0.4912 | 1.24 | 0.3215 | HMDB02092 |
| isovalerylglycine | 0.66 | 0.0028 | 1.18 | 0.3055 | 1.17 | 0.478 | HMDB00678 |
| N-6-trimethyllysine | 0.68 | 0.0029 | 0.88 | 0.1121 | 0.93 | 0.5685 | HMDB01325 |
| 2-hydroxyisobutyrate | 1.37 | 0.0029 | 1.27 | 0.0134 | 0.77 | 0.0064 | HMDB00729 |
| beta-hydroxypyruvate | 1.78 | 0.0031 | 0.99 | 0.74 | 0.78 | 0.0062 | HMDB01352 |
| pimelate (heptanedioate) | 0.61 | 0.0035 | 1.19 | 0.3425 | 1.12 | 0.7102 | HMDB00857 |
| glycine | 0.89 | 0.0036 | 0.79 | 0.0037 | 1.03 | 0.9682 | HMDB00123 |
| mannose | 0.55 | 0.004 | 0.82 | 0.3395 | 1.12 | 0.8406 | HMDB00169 |
| cysteine | 0.82 | 0.0052 | 0.88 | 0.0567 | 0.91 | 0.2935 | HMDB00574 |
| N-acetyltyrosine | 0.6 | 0.0052 | 0.91 | 0.8458 | 1.41 | 0.0199 | HMDB00866 |
| glutamine | 1.53 | 0.0061 | 0.92 | 0.4043 | 1.49 | 0.3348 | HMDB00641 |
| leucine | 1.28 | 0.0067 | 0.96 | 0.9327 | 1.04 | 0.7329 | HMDB00687 |
| indolelactate | 0.73 | 0.007 | 0.94 | 0.508 | 1.67 | 0.0254 | HMDB00671 |
| xanthine | 1.41 | 0.0073 | 1.06 | 0.6782 | 1.37 | 0.1721 | HMDB00292 |
| lactose | 0.58 | 0.0074 | 1.12 | 0.78 | 1.27 | 0.2407 | HMDB00186 |
| threonine | 0.86 | 0.0079 | 0.87 | 0.0163 | 1.21 | 0.6336 | HMDB00167 |
| kynurenine | 1.6 | 0.008 | 0.74 | 0.4686 | 1.25 | 0.5888 | HMDB00684 |
| sorbitol | 0.75 | 0.0087 | 3.42 | 0.7352 | 4.56 | 0.621 | HMDB00247 |
| 3-hydroxysebacate | 1.75 | 0.009 | 0.86 | 0.7823 | 0.75 | 0.1105 | HMDB00350 |
| 5-hydroxyindoleacetate | 0.7 | 0.0093 | 1.07 | 0.8213 | 1.13 | 0.7909 | HMDB00763 |
| pyroglutamine | 0.81 | 0.0103 | 0.87 | 0.1065 | 0.96 | 0.6105 | |
| azelate (nonanedioate) | 0.64 | 0.0107 | 0.8 | 0.1913 | 1.47 | 0.0155 | HMDB00784 |
| neopterin | 1.41 | 0.012 | 1.21 | 0.3553 | 1.38 | 0.0315 | HMDB00845 |
| gamma-glutamyltyrosine | 0.74 | 0.0125 | 0.99 | 0.6907 | 1.1 | 0.8961 | |
| 4-vinylphenol sulfate | 0.77 | 0.0128 | 1.01 | 0.877 | 1.11 | 0.7154 | HMDB04072 |
| dimethylglycine | 0.75 | 0.0135 | 0.85 | 0.0686 | 0.88 | 0.3711 | HMDB00092 |
| serine | 0.82 | 0.0138 | 0.82 | 0.0222 | 0.9 | 0.9516 | HMDB03406 |
| creatine | 0.36 | 0.015 | 1.16 | 0.6036 | 1.62 | 0.2614 | HMDB00064 |
| octanoylcarnitine | 1.29 | 0.0152 | 1.22 | 0.2376 | 0.86 | 0.249 | |
| 3-methoxytyrosine | 1.63 | 0.0174 | 1.64 | 0.1587 | 3.44 | 0.1716 | HMDB01434 |
| malate | 2.63 | 0.018 | 2.28 | 0.6561 | 2.02 | 0.8528 | HMDB00156 |
| mandelate | 0.8 | 0.0187 | 1.03 | 0.6199 | 1.1 | 0.2628 | HMDB00703 |
| aspartate | 0.82 | 0.0192 | 0.66 | 0.005 | 1.4 | 0.2923 | HMDB00191 |
| gamma-glutamylthreonine | 0.81 | 0.0196 | 0.91 | 0.0883 | 1.11 | 0.7569 | |
| 4-ureidobutyrate | 0.86 | 0.0234 | 0.98 | 0.5831 | 1.13 | 0.1905 | |
| valine | 1.25 | 0.0235 | 0.93 | 0.6915 | 1.08 | 0.6722 | HMDB00883 |
| alpha-ketoglutarate | 1.99 | 0.0241 | 1.47 | 0.3582 | 1.42 | 0.2569 | HMDB00208 |
| 5-acetylamino-6-amino-3- | 0.43 | 0.0263 | 0.89 | 0.6847 | 1.04 | 0.8541 | HMDB04400 |
| methyluracil | | | | | | | |
| 4-hydroxyphenylacetate | 0.69 | 0.0269 | 1.46 | 0.0015 | 1.28 | 0.3338 | HMDB00020 |
| gamma-glutamylphenylalanine | 1.34 | 0.0322 | 0.9 | 0.0659 | 1.14 | 0.8583 | HMDB00594 |
| isocitrate | 0.8 | 0.0331 | 0.8 | 0.1792 | 1.11 | 0.9539 | HMDB00193, |
| | | | | | | | HMDB01874 |
| threitol | 0.83 | 0.0371 | 0.87 | 0.842 | 0.78 | 0.3598 | HMDB04136 |
| pantothenate | 0.64 | 0.0396 | 1.12 | 0.4425 | 1.01 | 0.5022 | HMDB00210 |
| N6-carbamoylthreonyladenosine | 1.29 | 0.044 | 1.13 | 0.3033 | 1.19 | 0.2383 | |
| isoleucine | 1.24 | 0.048 | 0.88 | 0.3879 | 1.09 | 0.6039 | HMDB00172 |
| N-acetylglutamine | 1.41 | 0.0488 | 1.58 | 0.0168 | 1.27 | 0.3028 | HMDB06029 |
| androsterone sulfate | 1.25 | 0.0568 | 1.51 | 0.0454 | 0.97 | 0.4081 | HMDB02759 |
| N4-acetylcytidine | 1.23 | 0.0585 | 1.19 | 0.1462 | 1.19 | 0.0562 | HMDB05923 |
| galactitol (dulcitol) | 0.8 | 0.0603 | 1.06 | 0.4119 | 1.25 | 0.3608 | HMDB00107 |
| pro-hydroxy-pro | 1.24 | 0.0663 | 1.1 | 0.2669 | 1.13 | 0.2931 | HMDB06695 |
| lactate | 1.24 | 0.0667 | 0.39 | 3.29E−05 | 1.34 | 0.1663 | HMDB00190 |
| 1-methylurate | 0.84 | 0.0674 | 0.7 | 0.0816 | 1.01 | 0.7689 | HMDB03099 |
| indoleacetate | 1.42 | 0.0689 | 1.34 | 0.1364 | 1.32 | 0.592 | HMDB00197 |
| urate | 1.11 | 0.0734 | 0.94 | 0.3996 | 1.18 | 0.0807 | HMDB00289 |
| phenylalanine | 1.26 | 0.0758 | 1.21 | 0.1977 | 1.16 | 0.2046 | HMDB00159 |
| gamma-glutamylleucine | 0.77 | 0.0815 | 1.06 | 0.8816 | 0.96 | 0.6133 | HMDB11171 |
| 4-ethylphenylsulfate | 0.54 | 0.0829 | 0.67 | 0.8041 | 0.89 | 0.2725 | |
| carnosine | 0.36 | 0.0878 | 0.68 | 0.8209 | 0.72 | 0.6219 | HMDB00033 |
| homocitrulline | 0.84 | 0.0979 | 0.86 | 0.1723 | 1.01 | 0.4838 | HMDB00679 |
| 2-aminobutyrate | 1.14 | 0.0986 | 0.81 | 0.0271 | 0.76 | 0.3751 | HMDB00650 |
| 5-hydroxyhexanoate | 0.68 | 0.099 | 1.04 | 0.4115 | 1.11 | 0.6993 | HMDB00525 |
| isovalerylcarnitine | 0.64 | 0.1644 | 0.66 | 0.1875 | 0.64 | 0.0037 | HMDB00688 |
| glycocholate | 0.9 | 0.1771 | 1.1 | 0.9661 | 2.14 | 0.0079 | HMDB00138 |
| cholate | 0.6 | 0.2725 | 0.77 | 0.8537 | 2 | 0.0147 | HMDB00619 |
| 3-indoxyl sulfate | 0.92 | 0.3457 | 1.78 | 1.08E−06 | 1.52 | 0.0602 | HMDB00682 |
| proline | 1.1 | 0.3963 | 0.91 | 0.5784 | 1.39 | 0.0029 | HMDB00162 |
| mannitol | 0.94 | 0.5089 | 1.06 | 0.261 | 3 | 0.0017 | HMDB00765 |
| succinate | 1.11 | 0.6315 | 1.72 | 0.0024 | 1.14 | 0.9413 | HMDB00254 |
| pipecolate | 0.65 | 0.7311 | 1.06 | 0.5698 | 1.58 | 0.0706 | HMDB00070 |
| 3-hydroxyisobutyrate | 1.05 | 0.7472 | 1.16 | 0.0693 | 1.23 | 0.0014 | HMDB00336 |
| choline | 1.02 | 0.8127 | 0.72 | 0.0029 | 1.32 | 0.0174 | |
| adenosine | 1.07 | 0.8234 | 1.47 | 0.0004 | 1.15 | 0.8031 | HMDB00050 |
| N-acetylthreonine | 0.96 | 0.9472 | 1 | 0.822 | 1.23 | 0.0577 | |
| 7-ketodeoxycholate | 1.79 | 0.9864 | 2.15 | 0.2117 | 9.64 | 0.0009 | HMDB00391 |

The biomarkers were then used to create a statistical model to identify subjects having kidney cancer. Using Random Forest analysis, the biomarkers were used in a mathematical model to classify subjects as having kidney cancer or normal. The results of the Random Forest analysis show that the samples were classified with 93% prediction accuracy. The Confusion Matrix presented in Table 12 shows the number of samples predicted for each classification and the actual in each group (RCC or Normal). The “Out-of-Bag” (OOB) Error rate gives an estimate of how accurately new observations can be predicted using the Random Forest model (e.g., whether a sample is from a RCC subject or a normal subject). The OOB error was approximately 7%, and the model estimated that, when used on a new set of subjects, the identity of RCC subjects could be predicted 93% of the time and normal subjects could be predicted correctly 94% of the time.

TABLE 12
Results of Random Forest, RCC vs. Normal

| | | Predicted Group | | class. |
|---|---|---|---|---|
| | | RCC | Normal | Error |
| Actual | RCC | 45 | 3 | 0.067416 |
| Group | Normal | 6 | 83 | 0.0625 |

Based on the OOB Error rate of 7%, the Random Forest model that was created predicted whether a sample was from an individual with RCC with about 93% accuracy based on the levels of the biomarkers measured in samples from the subject. Exemplary biomarkers for distinguishing the groups are methyl-4-hydroxybenzoate, catechol-sulfate, glycerol, 2-hydroxyhippurate (salicylurate), N(2)-furoyl-glycine, 3-hydroxyphenylacetate, gulono 1,4-lactone, 2-isopropylmalate, 2-3-dihydroxyisovalerate, 1-2-propanediol, gluconate, cinnamoylglycine, phenylacetylglycine, sorbose, sucrose, adenosine 5′-monophosphate (AMP), hexanoylglycine, methyl-indole-3-acetate, 3-hydroxyhippurate, N6-methyladenosine, 4-hydroxy-2-oxoglutaric acid, alpha-CEHC-glucuronide, phenylpropinylglycine, vanillate, ethanolamine, galactose, adipate, 2-oxindole-3-acetate, 1, 3-7-trimethylurate, and 3-4-dihydroxyphenylacetate.

The Random Forest results demonstrated that by using the biomarkers, RCC subjects were distinguished from normal subjects with 94% sensitivity, 93% specificity, 88% PPV, and 97% NPV.

The biomarkers were used to create a statistical model to distinguish subjects having kidney cancer from those having prostate cancer. The biomarkers were evaluated using Random Forest analysis to classify subjects as having RCC or PCA. The Random Forest results show that the samples were classified with 80% prediction accuracy. The Confusion Matrix presented in Table 15 shows the number of samples predicted for each classification and the actual in each group (RCC or PCA). The “Out-of-Bag” (OOB) Error rate gives an estimate of how accurately new observations can be predicted using the Random Forest model (e.g., whether a sample is from a RCC subject or a PCA subject). The OOB error was approximately 20%, and the model estimated that, when used on a new set of subjects, the identity of RCC subjects could be predicted 77% of the time and PCA subjects could be predicted correctly 83% of the time and as presented in Table 13.

TABLE 13
Results of Random Forest, RCC vs. PCA

| | | Predicted Group | | class. |
|---|---|---|---|---|
| | | RCC | PCA | Error |
| Actual | RCC | 37 | 11 | 0.229167 |
| Group | PCA | 10 | 48 | 0.172414 |

Based on the OOB Error rate of 20%, the Random Forest model that was created predicted whether a sample was from an individual with RCC with about 80% accuracy based on the levels of the biomarkers measured in samples from the subject. The biomarkers that are the most important biomarkers for distinguishing the groups are gluconate, 1-2-propanediol, galactose, gulono 1,4-lactone, orotidine, quinate, 1, 3-7-trimethylurate, guanine, phenylacetylglutamine, mannitol, 2-oxindole-3-acetate, 1,3-aminopropyl-2-pyrrolidone, 1,3-dimethylurate, Isobar-glucuronate-galacturonate-5-keto-gluconate, glycocholate, azelate (nonanedioate), N-acetylthreonine, 7-ketodeoxycholate, 3-sialyllactose, isovalerylcarnitine, cholate, adenosine 5′-monophosphate (AMP), 2-3-butanediol, 2-hydroxyhippurate, pipecolate, N-acetylphenylalanine, 12-dehydrocholate, alpha-ketoglutarate, sulforaphane.

The Random Forest results demonstrated that by using the biomarkers, RCC subjects were distinguished from PCA subjects with 77% sensitivity, 83% specificity, 79% PPV, 81% NPV.

The biomarkers were used to create a statistical model to classify subjects as having kidney cancer from those having bladder cancer. The biomarkers were evaluated using Random Forest analysis to classify subjects as having RCC or BCA. The Random Forest results show that the samples were classified with 75% prediction accuracy. The Confusion Matrix presented in Table 14 shows the number of samples predicted for each classification and the actual in each group (RCC or BCA). The “Out-of-Bag” (OOB) Error rate gives an estimate of how accurately new observations can be predicted using the Random Forest model (e.g., whether a sample is from a RCC subject or a BCA subject). The OOB error was approximately 25%, and the model estimated that, when used on a new set of subjects, the identity of RCC subjects could be predicted 76% of the time and BCA subjects could be predicted correctly 73% of the time and as presented in Table 14.

TABLE 14
Results of Random Forest, RCC vs. BCA

| | | Predicted Group | | class. |
|---|---|---|---|---|
| | | RCC | BCA | Error |
| Acutal | RCC | 35 | 13 | 0.242424 |
| Group | BCA | 16 | 50 | 0.270833 |

Based on the OOB Error rate of 25%, the Random Forest model that was created predicted whether a sample was from an individual with RCC with about 75% accuracy based on the levels of the biomarkers measured in samples from the subject. The biomarkers that are the most important biomarkers for distinguishing the groups are 3-indoxyl-sulfate, methyl-indole-3-acetate, methyl-4-hydroxybenzoate, lactate, N(2)-furoyl-glycine, N6-methyladenosine, gamma-CEHC, glycerol, 2-3-butanediol, palmitoyl-sphingomyelin, succinate, 4-hydroxyphenylacetate, caffeate, imidazole-prpionate, beta-alanine, 4-androsten-3beta-17beta-diol-disulfate-2,5-methylthioadenosine, (MTA), N2-acetyllysine, sucrose, phenylacetylglycine, 4-androsten-3beta-17beta-diol-disulfate-1, cyclo-gly-pro, N-methyl-proline, catechol-sulfate, serine, vanillate, threonine, 21-hydroxypregnenolone-disulfate, adenosine 5′-monophosphate (AMP), phenylacetylglutamine.

The Random Forest results demonstrated that by using the biomarkers, RCC subjects were distinguished from BCA subjects with 73% sensitivity, 78% specificity, 69% PPV, and 79% NPV.

### Example 7

### Algorithm to Monitor Kidney Cancer Progression/Regression

Using the biomarkers for kidney cancer, an algorithm could be developed to monitor kidney cancer progression/regression in subjects. The algorithm, based on a panel of metabolite biomarkers from Tables 1, 2, 4, 8, 10 and/or 11, when used on a new set of patients, would assess and monitor a patient's progression/regression of kidney cancer. Using the results of this biomarker algorithm, a medical oncologist could assess the risk-benefit of surgery (i.e., full or partial nephrectomy), drug treatment or a watchful waiting approach.

The biomarker algorithm would monitor the levels of a panel of biomarkers for kidney cancer identified in Tables 1, 2, 4, 8, 10 and/or 11.

1. A method of diagnosing or aiding in diagnosing whether a subject has kidney cancer, comprising:
analyzing a biological sample from a subject to determine the level(s) of one or more biomarkers for kidney cancer in the sample, wherein the one or more biomarkers are selected from Tables 1, 2, 4 and/or 11, and wherein the sample is analyzed using mass spectrometry, and
comparing the level(s) of the one or more biomarkers in the sample to kidney cancer-positive and/or kidney cancer-negative reference levels of the one or more biomarkers in order to diagnose whether the subject has kidney cancer.

2. The method of claim 1, wherein the sample is also analyzed using one or more additional techniques selected from the group consisting of ELISA and antibody linkage.

3. The method of claim 1, wherein the method comprises analyzing the subject and a biological sample from the subject using a mathematical model comprising one or more biomarkers or measurements selected from Tables 1, 2, 4 and/or 11.

4. A method of monitoring progression/regression of kidney cancer in a subject comprising:
analyzing a first biological sample from a subject to determine the level(s) of one or more biomarkers for kidney cancer in the sample, and wherein the sample is analyzed using mass spectrometry, and wherein the one or more biomarkers are selected from Tables 1, 2, 4, 8, 10 and/or 11 and the first sample is obtained from the subject at a first time point;
analyzing a second biological sample from a subject to determine the level(s) of the one or more biomarkers, wherein the second sample is obtained from the subject at a second time point; and
comparing the level(s) of one or more biomarkers in the first sample to the level(s) of the one or more biomarkers in the second sample in order to monitor the progression/regression of kidney cancer in the subject.

5. The method of claim 4, wherein the method further comprises comparing the level(s) of one or more biomarkers in the first sample, the level(s) of one or more biomarkers in the second sample, and/or the results of the comparison of the level(s) of the one or more biomarkers in the first and second samples to kidney cancer-positive and/or kidney cancer-negative reference levels of the one or more biomarkers.

6. The method of claim 5, wherein the method comprises analyzing the subject and a biological sample from the subject using a mathematical model comprising one or more biomarkers or measurements selected from Tables 1 , 2, 4, 8, 10 and/or 11.

7-8. (canceled)

9. A method of distinguishing less aggressive kidney cancer from more aggressive kidney cancer in a subject having kidney cancer, comprising analyzing a biological sample from a subject to determine the level(s) of one or more biomarkers for kidney cancer in the sample, wherein the one or more biomarkers are selected from Table 10, and wherein the sample is analyzed using mass spectrometry, and
comparing the level(s) of the one or more biomarkers in the sample to less aggressive kidney cancer and/or more aggressive kidney cancer reference levels of the one or more biomarkers in order to determine the aggressiveness of the subject's kidney cancer.

10. The method of claim 9, wherein a mathematical model is used to distinguish less aggressive kidney cancer from more aggressive kidney cancer in a subject having kidney cancer.

11-26. (canceled)

27. The method of claim 1, wherein determining an RCC Score aids in the method thereof.

28. The method of claim 4, wherein determining an RCC Score aids in the method thereof.

29. The method of claim 9, wherein determining an RCC Score aids in the method thereof.
